SEQUENCE LISTING (42 sequences)

SEQ ID NO: 1 - RNA, 333 nt, synthetic construct
  source 1..333: mol_type = other RNA
gacattcagctgacccaaagccccgcctctctggctgtgagcctgggacaaagagccaccataagctgcaaagccagccaaagcgttgactatgacggagacagctacctgaactggtatcagcaaatccccggacagccccccaagctgctgatctacgacgccagcaacctggtgagcggcatccccccccgattcagcggaagcggcagcggaaccgacttcaccctcaacattcaccccgtggagaaggtcgacgccgccacctaccactgccagcagagcaccgaagatccctggaccttcggcggcggcaccaagctggaaatcaaa

SEQ ID NO: 2 - RNA, 372 nt, synthetic construct
  source 1..372: mol_type = other RNA
caggtccaactccaacaaagcggcgccgagctcgtcagacccggctcaagcgtcaaaatcagctgcaaagccagcggctacgccttcagcagctactggatgaactgggtgaagcagagacccggccaaggcctcgagtggattggacaaatctggcccggagacggagacaccaactacaacggaaaattcaaaggaaaagccacactgaccgccgacgaaagcagtagcaccgcctacatgcagctgtccagcctcgccagcgaagacagcgccgtgtacttctgcgccagacgggaaacaaccaccgtgggaagatactactacgccatggactactggggccagggaaccaccgtgaccgtgagcagc

SEQ ID NO: 3 - RNA, 318 nt, synthetic construct
  source 1..318: mol_type = other RNA
gacattcagctgacccaaagccccgccataatgagcgccagccccggagaaaaagtgaccatgacctgtagagccagcagcagcgtgagctatatgaactggtatcagcaaaaaagcggcaccagccccaagagatggatctacgacaccagcaaggtggccagcggagtgccctacagattctccggaagcggcagcggaacatcatacagcctgaccatcagcagcatggaagcagaagacgccgccacctactactgccagcagtggagctctaaccccctgaccttcggcgccggtaccaagctggagctgaaa

SEQ ID NO: 4 - RNA, 357 nt, synthetic construct
  source 1..357: mol_type = other RNA
gacattaagctgcagcagagcggcgccgagctggccagacctggagcttccgtgaaaatgagctgcaaaaccagcggctacacctttacaagatataccatgcactgggtgaaacagagacccggccaaggcctggagtggatcgggtacattaaccccagcagaggctacaccaactacaaccaaaaattcaaggacaaggccaccctgaccaccgacaaaagcagcagcaccgcctacatgcagctgagcagcctgaccagcgaagacagcgccgtgtactactgcgcccgatactacgacgaccactactgcctggactactggggccaaggcacaacactgaccgtgtccagc

SEQ ID NO: 5 - RNA, 321 nt, synthetic construct
  source 1..321: mol_type = other RNA
gacattcagatgacccagaccaccagcagcctcagcgccagcctgggcgacagagttaccatctcctgcagagccagccaagacataagcaagtatctcaactggtatcagcagaaacccgacggcaccgtgaagctgctcatctaccacaccagcagactccacagcggcgttcccagccggttcagcggaagcggctccggaacagactactccctgaccatcagcaacctcgaacaagaagacatcgcaacctacttctgccagcaaggcaacacactcccctacaccttcggaggcggcacaaagctggagatcacc

SEQ ID NO: 6 - RNA, 360 nt, synthetic construct
  source 1..360: mol_type = other RNA
gaggtgaagctgcaggagagcgggccaggactggtggcccctagccagagcctgagtgtgacatgtacagtgagcggagtgagcctgcccgactacggagtgagctggattagacagccacctagaaaaggactggagtggctgggagtgatttggggcagcgagaccacatactacaactctgccctgaagagcaggctgaccattataaaggacaattcaaagagccaggtgttcctgaagatgaatagcctgcagaccgatgataccgctatctactactgcgccaagcactactactatggcggaagctacgccatggactactggggacagggcaccagcgtcaccgtgagcagc

SEQ ID NO: 7 - RNA, 327 nt, synthetic construct
  source 1..327: mol_type = other RNA
cagaccgtcgtgacccaagagcccagcctcaccgtgagccccggcggaacagtgaccctcacctgcggaagcagcacaggagccgtgaccagcggatactaccccaactgggtccaacagaaacccggccaagcccccagaggcctgatcggaggaaccaagtttctcgcccccggcacacccgcaagattcagcggaagcctgctcggaggcaaggccgccctgaccctgagcggagtgcaacctgaagacgaggcagagtactactgcgctctgtggtactccaacagatgggtgttcggaggaggcacaaagctgaccgtcctg

SEQ ID NO: 8 - RNA, 375 nt, synthetic construct
  source 1..375: mol_type = other RNA
gaggtgcagctggtggagagcggggggggactggtgcagccaggaggaagcctgaagctgagctgtgccgcaagcgggttcacatttaataagtacgcaatgaactgggtgagacaggctcctggcaaaggactggagtgggtggctagaattagaagcaagtacaacaactacgccacatactatgccgacagtgtgaaggaccggttcaccataagcagagacgatagcaagaataccgcttacctgcaaatgaacaacctgaagaccgaggacaccgccgtctactactgtgtgaggcacggaaacttcggcaacagctacatctcctactgggcctactggggacaagggaccctggtgaccgtcagcagc

SEQ ID NO: 9 - RNA, 318 nt, synthetic construct
  source 1..318: mol_type = other RNA
gagaatgtgctgacccagagccccgccataatgagcgccagcccaggagagaaggtgacaatgacatgcagcgcaagcagcagcgtgagttacatgcactggtaccagcagaagagcagcaccagcccaaagctctggatctacgacaccagcaaactggccagtggagtgcccggcagattcagtggaagtggaagcggaaacagccacttcctgactatcagcagcatggaagccgaagacgtcgccacctactactgctttcagggaagcgtgtaccccttcaccttcggaagcggtaccaagctggagatcaag

SEQ ID NO: 10 - RNA, 360 nt, synthetic construct
  source 1..360: mol_type = other RNA
caggtcaccctcaaagagtcaggccccggcatcctccaacccagccaaaccctcagcctcacatgcagcttcagcggcttcagcctctccaccagcggcatgggcgtcggctggatcagacagccctccggaaaaggcctggaatggctcgcccacatctggtgggacgacgacaaaagatacaaccccgccctgaagtcccggctcaccatcagtaaagacaccagctccaaccaagtcttcctcaagatcgcctctgtggacaccgccgacaccgccgcttattactgcgccagaatggaactctggtcctactacttcgactactggggacaaggcaccaccctcaccgtctcctcc

SEQ ID NO: 11 - RNA, 84 nt, synthetic construct
  source 1..84: mol_type = other RNA
aaactacaagacagacttgcaaaagaaggcatgcacagctcagcactgctctgttgcctggtcctcctgactggggtgagggcc

SEQ ID NO: 12 - RNA, 60 nt, synthetic construct
  source 1..60: mol_type = other RNA
ggtggcggagggtctggtggtggaggatcagggggtggaggttcaggaggcgggggaagt

SEQ ID NO: 13 - RNA, 15 nt, synthetic construct
  source 1..15: mol_type = other RNA
ggtggcggtggatct

SEQ ID NO: 14 - RNA, 18 nt, synthetic construct
  source 1..18: mol_type = other RNA
caccatcaccatcatcac

SEQ ID NO: 15 - DNA, 333 bp, synthetic construct
  source 1..333: mol_type = other DNA
gacattcagctgacccaaagccccgcctctctggctgtgagcctgggacaaagagccaccataagctgcaaagccagccaaagcgttgactatgacggagacagctacctgaactggtatcagcaaatccccggacagccccccaagctgctgatctacgacgccagcaacctggtgagcggcatccccccccgattcagcggaagcggcagcggaaccgacttcaccctcaacattcaccccgtggagaaggtcgacgccgccacctaccactgccagcagagcaccgaagatccctggaccttcggcggcggcaccaagctggaaatcaaa

SEQ ID NO: 16 - DNA, 372 bp, synthetic construct
  source 1..372: mol_type = other DNA
caggtccaactccaacaaagcggcgccgagctcgtcagacccggctcaagcgtcaaaatcagctgcaaagccagcggctacgccttcagcagctactggatgaactgggtgaagcagagacccggccaaggcctcgagtggattggacaaatctggcccggagacggagacaccaactacaacggaaaattcaaaggaaaagccacactgaccgccgacgaaagcagtagcaccgcctacatgcagctgtccagcctcgccagcgaagacagcgccgtgtacttctgcgccagacgggaaacaaccaccgtgggaagatactactacgccatggactactggggccagggaaccaccgtgaccgtgagcagc

SEQ ID NO: 17 - DNA, 318 bp, synthetic construct
  source 1..318: mol_type = other DNA
gacattcagctgacccaaagccccgccataatgagcgccagccccggagaaaaagtgaccatgacctgtagagccagcagcagcgtgagctatatgaactggtatcagcaaaaaagcggcaccagccccaagagatggatctacgacaccagcaaggtggccagcggagtgccctacagattctccggaagcggcagcggaacatcatacagcctgaccatcagcagcatggaagcagaagacgccgccacctactactgccagcagtggagctctaaccccctgaccttcggcgccggtaccaagctggagctgaaa

SEQ ID NO: 18 - DNA, 357 bp, synthetic construct
  source 1..357: mol_type = other DNA
gacattaagctgcagcagagcggcgccgagctggccagacctggagcttccgtgaaaatgagctgcaaaaccagcggctacacctttacaagatataccatgcactgggtgaaacagagacccggccaaggcctggagtggatcgggtacattaaccccagcagaggctacaccaactacaaccaaaaattcaaggacaaggccaccctgaccaccgacaaaagcagcagcaccgcctacatgcagctgagcagcctgaccagcgaagacagcgccgtgtactactgcgcccgatactacgacgaccactactgcctggactactggggccaaggcacaacactgaccgtgtccagc

SEQ ID NO: 19 - DNA, 321 bp, synthetic construct
  source 1..321: mol_type = other DNA
gacattcagatgacccagaccaccagcagcctcagcgccagcctgggcgacagagttaccatctcctgcagagccagccaagacataagcaagtatctcaactggtatcagcagaaacccgacggcaccgtgaagctgctcatctaccacaccagcagactccacagcggcgttcccagccggttcagcggaagcggctccggaacagactactccctgaccatcagcaacctcgaacaagaagacatcgcaacctacttctgccagcaaggcaacacactcccctacaccttcggaggcggcacaaagctggagatcacc

SEQ ID NO: 20 - DNA, 360 bp, synthetic construct
  source 1..360: mol_type = other DNA
gaggtgaagctgcaggagagcgggccaggactggtggcccctagccagagcctgagtgtgacatgtacagtgagcggagtgagcctgcccgactacggagtgagctggattagacagccacctagaaaaggactggagtggctgggagtgatttggggcagcgagaccacatactacaactctgccctgaagagcaggctgaccattataaaggacaattcaaagagccaggtgttcctgaagatgaatagcctgcagaccgatgataccgctatctactactgcgccaagcactactactatggcggaagctacgccatggactactggggacagggcaccagcgtcaccgtgagcagc

SEQ ID NO: 21 - DNA, 327 bp, synthetic construct
  source 1..327: mol_type = other DNA
cagaccgtcgtgacccaagagcccagcctcaccgtgagccccggcggaacagtgaccctcacctgcggaagcagcacaggagccgtgaccagcggatactaccccaactgggtccaacagaaacccggccaagcccccagaggcctgatcggaggaaccaagtttctcgcccccggcacacccgcaagattcagcggaagcctgctcggaggcaaggccgccctgaccctgagcggagtgcaacctgaagacgaggcagagtactactgcgctctgtggtactccaacagatgggtgttcggaggaggcacaaagctgaccgtcctg

SEQ ID NO: 22 - DNA, 375 bp, synthetic construct
  source 1..375: mol_type = other DNA
gaggtgcagctggtggagagcggggggggactggtgcagccaggaggaagcctgaagctgagctgtgccgcaagcgggttcacatttaataagtacgcaatgaactgggtgagacaggctcctggcaaaggactggagtgggtggctagaattagaagcaagtacaacaactacgccacatactatgccgacagtgtgaaggaccggttcaccataagcagagacgatagcaagaataccgcttacctgcaaatgaacaacctgaagaccgaggacaccgccgtctactactgtgtgaggcacggaaacttcggcaacagctacatctcctactgggcctactggggacaagggaccctggtgaccgtcagcagc

SEQ ID NO: 23 - DNA, 318 bp, synthetic construct
  source 1..318: mol_type = other DNA
gagaatgtgctgacccagagccccgccataatgagcgccagcccaggagagaaggtgacaatgacatgcagcgcaagcagcagcgtgagttacatgcactggtaccagcagaagagcagcaccagcccaaagctctggatctacgacaccagcaaactggccagtggagtgcccggcagattcagtggaagtggaagcggaaacagccacttcctgactatcagcagcatggaagccgaagacgtcgccacctactactgctttcagggaagcgtgtaccccttcaccttcggaagcggtaccaagctggagatcaag

SEQ ID NO: 24 - DNA, 360 bp, synthetic construct
  source 1..360: mol_type = other DNA
caggtcaccctcaaagagtcaggccccggcatcctccaacccagccaaaccctcagcctcacatgcagcttcagcggcttcagcctctccaccagcggcatgggcgtcggctggatcagacagccctccggaaaaggcctggaatggctcgcccacatctggtgggacgacgacaaaagatacaaccccgccctgaagtcccggctcaccatcagtaaagacaccagctccaaccaagtcttcctcaagatcgcctctgtggacaccgccgacaccgccgcttattactgcgccagaatggaactctggtcctactacttcgactactggggacaaggcaccaccctcaccgtctcctcc

SEQ ID NO: 25 - DNA, 84 bp, synthetic construct
  source 1..84: mol_type = other DNA
aaactacaagacagacttgcaaaagaaggcatgcacagctcagcactgctctgttgcctggtcctcctgactggggtgagggcc

SEQ ID NO: 26 - DNA, 60 bp, synthetic construct
  source 1..60: mol_type = other DNA
ggtggcggagggtctggtggtggaggatcagggggtggaggttcaggaggcgggggaagt

SEQ ID NO: 27 - DNA, 15 bp, synthetic construct
  source 1..15: mol_type = other DNA
ggtggcggtggatct

SEQ ID NO: 28 - DNA, 18 bp, synthetic construct
  source 1..18: mol_type = other DNA
caccatcaccatcatcac

SEQ ID NO: 29 - protein, 111 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 30 - protein, 124 aa, synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 31 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 32 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 33 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 34 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 35 - protein, 109 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 36 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 37 - protein, 106 aa, synthetic construct
ENVLTQSPAIMSASPGEKVTMTCSASSSVSYMHWYQQKSSTSPKLWIYDTSKLASGVPGRFSGSGSGNSHFLTISSMEAEDVATYYCFQGSVYPFTFGSGTKLEIK

SEQ ID NO: 38 - protein, 120 aa, synthetic construct
QVTLKESGPGILQPSQTLSLTCSFSGFSLSTSGMGVGWIRQPSGKGLEWLAHIWWDDDKRYNPALKSRLTISKDTSSNQVFLKIASVDTADTAAYYCARMELWSYYFDYWGQGTTLTVSS

SEQ ID NO: 39 - protein, 28 aa, synthetic construct
KLQDRLAKEGMHSSALLCCLVLLTGVRA

SEQ ID NO: 40 - protein, 20 aa, synthetic construct
GGGGSGGGGSGGGGSGGGGS

SEQ ID NO: 41 - protein, 5 aa, synthetic construct
GGGGS

SEQ ID NO: 42 - protein, 6 aa, synthetic construct
HHHHHH

(19) United States
(12) Patent Application Publication
(10) Pub. No.: US 2026/0335351 A1
(43) Pub. Date: Oct. 29, 2026

(54) LIPID NANOPARTICLES FOR IMMUNOTHERAPY

(71) Applicants: Trustees of Tufts College, Medford, MA (US); Hopewell Therapeutics, Inc., Woburn, MA (US)

(72) Inventors: Xin Kai, Winchester, MA (US); Qiaobing Xu, Medford, MA (US)

(21) Appl. No.: 18/998,674

(22) PCT Filed: Jul. 27, 2023
(86) PCT No.: PCT/US23/28864

§ 371 Date: Jan. 27, 2025

Related U.S. Application Data
(60) Provisional application No. 63/392,800, filed on Jul. 27, 2022.
(60) Provisional application No. 63/510,616, filed on Jun. 27, 2023.

(51) Int. Cl. A61K 31/7105; A61K 9/00; A61K 9/51; A61K 38/00; A61K 47/18; A61K 47/20; A61P 35/00; C07K 16/28

(52) U.S. Cl. CPC A61K 31/7105; A61K 9/0019; A61K 9/5123; A61K 9/5192; A61K 38/00; A61K 47/18; A61K 47/20; A61P 35/00; C07K 16/2803; C07K 16/2809; C07K 2317/31; C07K 2317/622

(57) ABSTRACT

Disclosed are compositions and methods related to lipid nanoparticles (LNPs) comprising ionizable lipids. The LNPs can comprise nucleic acid sequences encoding therapeutic peptides for immunotherapy, for example, bispecific antibodies or antigen binding fragments thereof.

### CROSS REFERENCE TO RELATED APPLICATIONS

This application claims the benefit of priority to U.S. Provisional Application No. 63/392,800, filed Jul. 27, 2022; and U.S. Provisional Application No. 63/510,616, filed Jun. 27, 2023; each of which is incorporated herein by reference in its entirety.

### BACKGROUND

Synthetic mRNA provides a template for the synthesis of any given peptide, protein or protein fragment and lends itself to a broad range of pharmaceutical applications, including different modalities of cancer immunotherapy. To function in vivo, mRNA requires safe, effective, and stable delivery systems that protect the nucleic acid from degradation and that allow cellular uptake and mRNA release. Lipid nanoparticle-mRNA formulations have been developed and are under clinical evaluation for the prevention and treatment of viral infections, cancer, and genetic diseases.

### SUMMARY

Provided herein are pharmaceutical compositions and methods related to lipid nanoparticles delivering nucleic acid molecules encoding therapeutic peptides that are configured to bind to a B-cell antigen. In some embodiments, the pharmaceutical composition comprises: a) a synthetic nucleic acid molecule encoding a therapeutic peptide, wherein the therapeutic peptide is configured to bind to a B-cell antigen; and b) a plurality of lipid nanoparticles; wherein the synthetic nucleic acid molecule is encapsulated within at least one of the plurality of lipid nanoparticles.

In some embodiments, the B-cell antigen is selected from the group consisting of a memory B-cell antigen, a naïve B-cell antigen, a plasmablast antigen, or a plasma cell antigen, and any combinations thereof. In some embodiments, the B-cell antigen is selected from the group consisting of cluster of differentiation (CD) 10, CD19, CD20, CD22, CD27, CD32b, CD38, CD40, B-cell maturation antigen (BCMA), B-cell activating factor receptor (BAFFR), CD138, CD5, and any combinations thereof. In some embodiments, the B-cell antigen comprises CD19. In some embodiments, the therapeutic peptide is capable of binding to the B-cell antigen with higher affinity than other antigens. In some embodiments, the therapeutic peptide binds to a second antigen. In some embodiments, the second antigen comprises an immune cell antigen or a tumor antigen. In some embodiments, the immune cell antigen comprises a T-cell antigen, a Treg cell antigen, or a natural killer cell (NK-cell) antigen. In some embodiments, the second antigen comprises CD3 and the therapeutic peptide is configured to bind to CD3.

In some embodiments, the therapeutic peptide comprises an antibody or an antigen-binding fragment thereof. In some embodiments, the antibody or the antigen-binding fragment thereof comprises a bispecific antibody, a multispecific antibody, or a chimeric antigen receptor (CAR). In some embodiments, the bispecific antibody or the antigen-binding fragment thereof comprises a bispecific T-cell engager (BiTE). In some embodiments, the BiTE is configured to bind to CD19 and CD 3. In some embodiments, the therapeutic peptide comprises an FDA-approved drug, wherein the FDA-approved drug is configured to bind to a B-cell antigen. In some embodiments, the FDA-approved drug comprises blinatumomab, inebilizumab, loncastuximab, or tafasitamab. In some embodiments, the synthetic nucleic acid molecule further comprises a regulatory nucleic acid sequence. In some embodiments, the regulatory nucleic acid sequence comprises a promoter or a signal peptide. In some embodiments, the promoter comprises a tissue-specific promoter.

In some embodiments, the synthetic nucleic acid molecule comprises a synthetic ribonucleic acid sequence (RNA). In some embodiments, the synthetic RNA comprises chemically modified nucleic acids, and wherein the synthetic RNA comprises an improved stability. In some embodiments, the synthetic RNA comprises N6-methyladenosine (m6A), N6,2′-O-dimethyladenosine (m6Am), 8-oxo-7,8-dihydroguanosine (8-oxoG), pseudouridine (Ψ), 5-methylcytidine (m5C), or N4-acetylcytidine (ac4C), or any combinations thereof. In some embodiments, the synthetic RNA comprises a 5′ cap.

In some embodiments, the synthetic RNA comprises, in a 5′ to 3′ direction: a) a 5′ untranslated region (5′ UTR) coding region; b) a signal peptide coding region; c) a therapeutic peptide coding region; d) a 3′ untranslated region (3′ UTR) coding region; and e) a poly A tail coding region. In some embodiments, the signal peptide coding region comprises the sequence of SEQ ID NO: 11. In some embodiments, the synthetic RNA further comprises a linker coding region. In some embodiments, the linker coding region comprises the sequence of SEQ ID NO: 12 or SEQ ID NO: 13. In some embodiments, the therapeutic peptide coding region encodes a variable light (VL) chain by a sequence that is at least 90%, 95%, 98%, or 99% identical to SEQ ID NOS: 1, 3, 5, 7, or 9; and a variable heavy (VH) chain encoding by a sequence that is at least 90%, 95%, 98%, or 99% identical to SEQ ID NOS: 2, 4, 6, 8, or 10. In some embodiments, the therapeutic peptide coding region encodes a VL chain and a VH chain by the sequence of 1) SEQ ID NOs: 1 and 2; 2) SEQ ID NOs: 3 and 4; 3) SEQ ID NOs: 5 and 6; 4) SEQ ID NOs: 7 and 8; or 5) SEQ ID NOs: 9 and 10. In some embodiments, the therapeutic peptide coding region comprises nucleic acid sequences having at least 90%, 95%, 98%, or 99% sequence identity to sequences selected from the group consisting of: 1) SEQ ID NOs: 1 and 2; 2) SEQ ID NOs: 5 and 6; or 3) SEQ ID NOs: 9 and 10, wherein the nucleic acid sequences encode an anti-CD19 antibody or anti-CD19 binding fragments thereof. In some embodiments, the therapeutic peptide coding region comprises nucleic acid sequences consisting of: 1) SEQ ID NOs: 3 and 4; or 2) SEQ ID NOs: 7 and 8, wherein the nucleic acid sequences encode an anti-CD3 antibody or anti-CD3 binding fragments thereof. In some embodiments, the therapeutic peptide coding region comprises nucleic acid sequences having at least 90%, 95%, 98%, or 99% sequence identity to 1) SEQ ID NOs: 3 and 4; or 2) SEQ ID NOs: 7 and 8, wherein the nucleic acid sequences encode an anti-CD3 antibody or anti-CD3 binding fragments thereof.

In some embodiments, the synthetic RNA further encodes a protein tag. In some embodiments, the protein tag comprises a histidine tag, a flag tag or a hemagglutinin tag. In some embodiments, the therapeutic peptide coding region comprises, in a 5′ to 3′ direction: a) an anti-CD19 light chain coding region; b) an anti-CD19 heavy chain coding region; c) an anti-CD3 heavy chain coding region; and d) an anti-CD3 light chain coding region. In some embodiments, the anti-CD19 light chain coding region comprises the sequence of SEQ ID NOs: 1, 3, 5, or 9. In some embodiments, the anti-CD19 heavy chain coding region comprises the sequence of SEQ ID NOs: 2, 4, 6, or 10. In some embodiments, the anti-CD19 light chain coding region comprises nucleic acid sequences having at least 90%, 95%, 98%, or 99% sequence identity to SEQ ID NOs: 1, 3, 5, or 9. In some embodiments, the anti-CD19 heavy chain coding region comprises nucleic acid sequences having at least 90%, 95%, 98%, or 99% sequence identity to SEQ ID NOs: 2, 4, 6, or 10. In some embodiments, the anti-CD3 light chain coding region comprises the sequence of SEQ ID NO: 7. In some embodiments, the anti-CD3 light chain coding region comprises nucleic acid sequences having at least 90%, 95%, 98%, or 99% sequence identity to SEQ ID NO: 7. In some embodiments, the anti-CD3 heavy chain coding region comprises the sequence of SEQ ID NO: 8. In some embodiments, the anti-CD3 heavy chain coding region comprises nucleic acid sequences having at least 90%, 95%, 98%, or 99% sequence identity to SEQ ID NO: 8. In some embodiments, the anti-CD19 light chain coding region comprises SEQ ID NO: 1 and the anti-CD19 heavy chain coding region comprises SEQ ID NO: 2; wherein the anti-CD3 light chain coding region comprises SEQ ID NO: 3 and the anti-CD3 heavy chain coding region comprises SEQ ID NO: 4. In some embodiments, the anti-CD19 light chain coding region comprises SEQ ID NO: 5 and the anti-CD19 heavy chain coding region comprises SEQ ID NO: 6; wherein the anti-CD3 light chain coding region comprises SEQ ID NO: 7 and the anti-CD3 heavy chain coding region comprises SEQ ID NO: 8. In some embodiments, the anti-CD19 light chain coding region comprises SEQ ID NO: 9 and the anti-CD19 heavy chain coding region comprises SEQ ID NO: 10; wherein the anti-CD3 light chain coding region comprises SEQ ID NO: 7 and the anti-CD3 heavy chain coding region comprises SEQ ID NO: 8. In some embodiments, the anti-CD19 heavy chain coding region comprises a nucleotide sequence that encodes a light chain of blinatumomab, inebilizumab, loncastuximab, or tafasitamab. In some embodiments, the anti-CD19 heavy chain coding region comprises a nucleotide sequence that encodes a heavy chain of blinatumomab, inebilizumab, loncastuximab, or tafasitamab.

In some embodiments, the synthetic RNA comprises a single strand synthetic RNA. In some embodiments, the lipid nanoparticle targets a tissue or an organ upon administration to a subject. In some embodiments, the tissue or the organ comprises a liver. In some embodiments, the tissue or the organ comprises a lymphoid organ. In some embodiments, the lymphoid organ comprises bone marrow, spleen, or lymph nodes. In some embodiments, the lipid nanoparticle targets a target cell upon administration to a subject. In some embodiments, the target cell comprises a hepatocyte, a lymphocyte, a leukocyte, a myeloid cell, or a hematopoietic stem cell. In some embodiments, the target cell comprises a B cell, and wherein the B cell comprises a plasmablast, a plasma cell, or a memory B cell. In some embodiments, the target cell comprises a T cell. In some embodiments, the target cell comprises a cell in a systemic circulation of the subject. In some embodiments, the target cell comprises a cell within a tissue or an organ of the subject.

In some embodiments, upon administration to a subject, the lipid nanoparticle has a faster rate of clearance compared to other lipid nanoparticles. In some embodiments, upon administration to a subject, the therapeutic peptide encoded by the synthetic nucleic acid sequence of the pharmaceutical composition has a longer half-life compared to a corresponding therapeutic peptide comprised in another pharmaceutical composition. In some embodiments, upon administration to a subject, the therapeutic peptide encoded by the synthetic nucleic acid sequence of the pharmaceutical composition has a half-life of at least 2 hours, at least 2.5 hours, at least 3 hours, at least 3.5 hours, at least 4 hours, at least 4.5 hours, at least 5 hours, at least 5.5 hours, at least 6 hours, at least 6.5 hours, at least 7 hours, at least 7.5 hours, at least 8 hours, at least 8.5 hours, at least 9 hours, at least 9.5 hours, at least 10 hours, at least 12 hours, at least 16 hours, at least 18 hours, at least 24 hours, at least 1.5 days, at least 2 days, at least 2.5 days, at least 3 days, at least 3.5 days, at least 4 days, at least 4.5 days, at least 5 days, at least 5.5 days, at least 6 days, at least 6.5 days, at least 7 days, at least 7.5 days, at least 8 days, at least 8.5 days, at least 9 days, at least 9.5 days, at least 10 days, at least 11 days, at least 12 days, at least 13 days, at least 14 days, at least 15 days, or more days.

In some embodiments, upon administration to a subject, the therapeutic peptide encoded by the synthetic nucleic acid sequence has a larger area under curve (AUC) compared to an equivalent dose of a corresponding therapeutic peptide of another pharmaceutical composition.

In some embodiments, upon administration to a subject, the therapeutic peptide encoded by the synthetic nucleic acid sequence has an AUC that is at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, or 500% or more larger compared to an equivalent dose of a corresponding therapeutic peptide of another pharmaceutical composition.

In some embodiments, upon administration to a subject, the therapeutic peptide encoded by the synthetic nucleic acid sequence results in prolonged B-cell depletion compared to an equivalent dose of a corresponding therapeutic peptide of another pharmaceutical composition.

In some embodiments, the prolonged B-cell depletion comprises B cell depletion for at least 6 hours, at least 12 hours, at least 18 hours, at least 24 hours, at least 3 days, at least 4 days, at least 5 days, at least 6 days, at least 7 days, at least 8 days, at least 9 days, at least 10 days, at least 15 days, at least 17.5 days, at least 20 days, at least 25 days, at least 27.5 days, at least 30 days, at least 35 days, at least 37.5 days, at least 40 days, at least 45 days, at least 47.5 days, at least 50 days, at least 55 days, at least 57.5 days, at least 60 days, at least 65 days, at least 70 days, at least 75 days, at least 80 days, at least 85 days, at least 90 days, at least 95 days, at least 100 days, at least 105 days, at least 110 days, or at least 120 or more days.

In some embodiments, upon administration to a subject the therapeutic peptide encoded by the synthetic nucleic acid sequence of the pharmaceutical composition results in depletion of at least 50%, at least 55%, at least 60%, at least 65%, at least 70%, at least 75%, at least 80%, at least 85%, at least 90%, at least 95%, or at least 99% or more of circulating B-cells in a subject. In some embodiments, upon administration to a subject the therapeutic peptide encoded by the synthetic nucleic acid sequence of the pharmaceutical composition results in depletion of at least 50%, at least 55%, at least 60%, at least 65%, at least 70%, at least 75%, at least 80%, at least 85%, at least 90%, at least 95%, or at least 99% or more of tissue B-cells in a subject. In some embodiments, upon administration to a subject the therapeutic peptide encoded by the synthetic nucleic acid sequence of the pharmaceutical composition results in depletion of at least 50%, at least 55%, at least 60%, at least 65%, at least 70%, at least 75%, at least 80%, at least 85%, at least 90%, at least 95%, or at least 99% or more of naive B-cells in a subject. In some embodiments, upon administration to a subject the therapeutic peptide encoded by the synthetic nucleic acid sequence of the pharmaceutical composition results in depletion of at least 50%, at least 55%, at least 60%, at least 65%, at least 70%, at least 75%, at least 80%, at least 85%, at least 90%, at least 95%, or at least 99% or more of mature B-cells in a subject. In some embodiments, the mature B-cell comprises a memory B-cell, a plasma cell, or a plasmablast.

In some embodiments, upon administration to a subject the therapeutic peptide encoded by the synthetic nucleic acid sequence of the pharmaceutical composition results in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200% or more B-cell depletion compared to an equivalent dose of a corresponding therapeutic peptide of another pharmaceutical composition.

In some embodiments, the pharmaceutical composition further comprises a pharmaceutically acceptable excipient. In some embodiments, the pharmaceutically acceptable excipient comprises a solution suitable for injections into a subject. In some embodiments, the pharmaceutical composition further comprises a small molecule drug. In some embodiments, the small molecule drug comprises an agent for chemotherapy. In some embodiments, the subject comprises a mammal. In some embodiments, the mammal comprises a human, a non-human primate, or a rodent. In some embodiments, upon administration to the subject the pharmaceutical composition has zero to minimum toxicity. In some embodiments, the toxicity comprises transient elevation in cytokines or liver enzymes. In some embodiments, the toxicity comprises mild inflammation or mild hepatotoxicity. In some embodiments, after being administered to the subject the pharmaceutical composition resulted in activation of CD69+ T cells.

In some embodiments, the lipid nanoparticle comprises a lipid composition; wherein the lipid composition comprises an ionizable lipid or a pharmaceutically acceptable salt thereof; wherein the ionizable lipid comprises an amine head group and at least one hydrophobic tail RLipid having a structure of

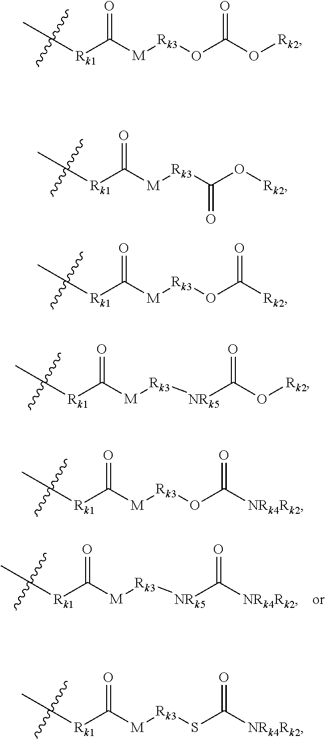

or a pharmaceutically acceptable salt thereof;
wherein
Rk1 is independently a C1-C12 bivalent aliphatic or heteroaliphatic radical;
Rk3 is independently a C1-C12 alkyl, C2-C12 alkenyl, C2-C12 alkynyl, C3-C12 cycloalkyl, C3-C12 heterocycloalkyl, aryl, or heteroaryl;
Rk2 is independently a C1-C20 alkyl, C2-C20 alkenyl, C2-C20 alkynyl, C3-C20 cycloalkyl, C1-C20 heteroalkyl, C3-C20 heterocycloalkyl, aryl, or heteroaryl;
Rk4 and Rk5 are each independently H or C1-C12 bivalent aliphatic radical; and
M is 0 or NRk6, wherein Rk6 is H or C1-C12 aliphatic radical.

In some embodiments, the amine head group is represented by:

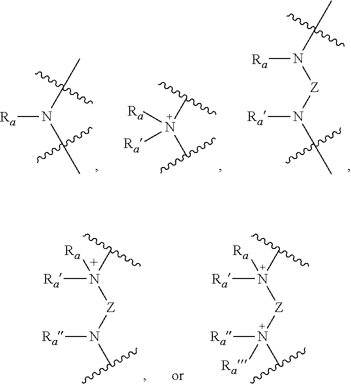

wherein Ra, Ra′, Ra″, and Ra′″ are each independently, H, C1-20 alkyl, C2-C20 alkenyl, C2-C20 alkynyl, C3-C20 cycloalkyl or heterocycloalkyl, C1-C20 heteroalkyl, C3-C20 aryl or heteroaryl, or a RLipid; and Z is a C1-C20 bivalent aliphatic radical, a C1-C20 bivalent heteroaliphatic radical, a bivalent aryl radical, or a bivalent heteroaryl radical.

In some embodiments, the ionizable lipid is represented by Formula (II):

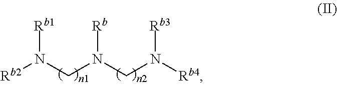

or a pharmaceutically acceptable salt thereof, wherein:
Rb is a substituted or unsubstituted alkyl;
n1 and n2 are each independently 1, 2, 3, 4, 5, or 6; and
Rb1, Rb2, Rb3 and Rb4 are each independently H or RLipid,
wherein at least one of Rb1, Rb2, Rb3 and Rb4 is not H.

In some embodiments, the amine head group is selected from the group consisting of

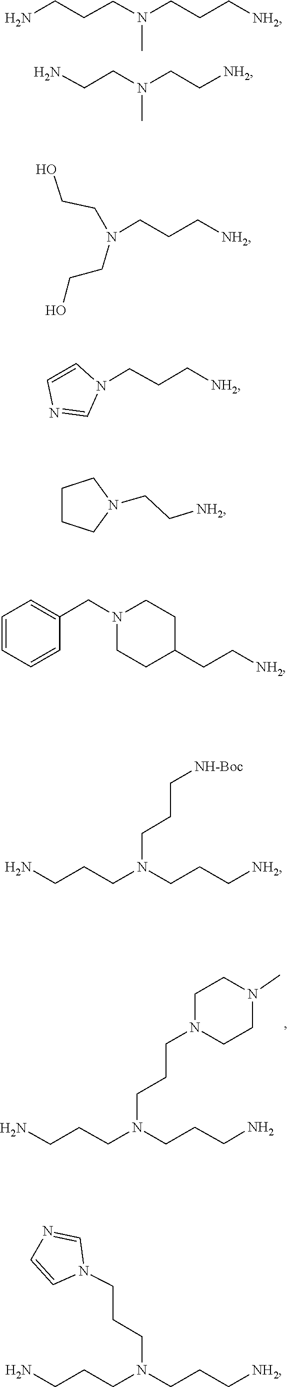

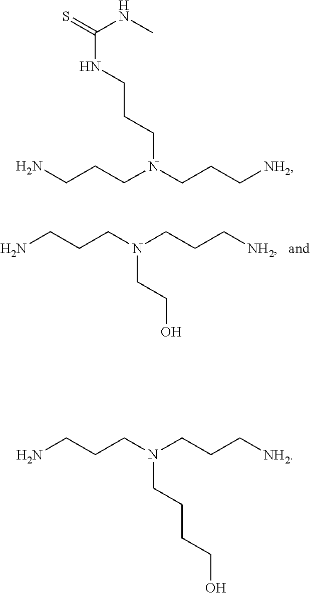 [SMILES: CNC(=S)NCCCN(CCCN)CCCN ; NCCCN(CCO)CCCN ; "and" ; NCCCN(CCCN)CCCCO]

In some embodiments, the at least one hydrophobic tail comprises,

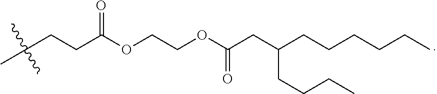 [SMILES: *CCC(=O)OCCOC(=O)CC(CCCC)CCCCCC]

In some embodiments, the ionizable lipid comprises

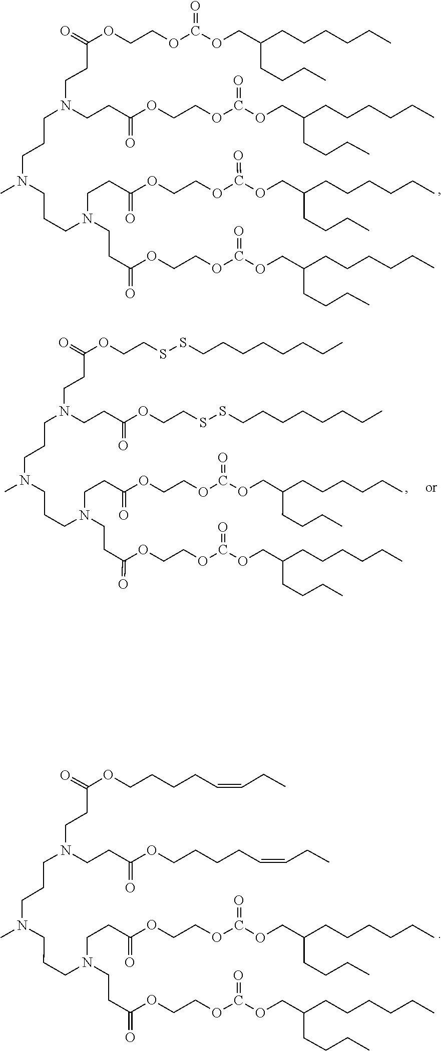

In some embodiments, the lipid composition further comprises a steroid. In some embodiments, the steroid comprises cholesterol or a cholesterol derivative. In some embodiments, the lipid composition further comprises a helper lipid. In some embodiments, the helper lipid comprises phospholipids or zwitterionic lipids comprising 1,2-dioleoyl-sn-glycero-3-phosphoethanolamine (DOPE) or 1,2-dioleoyl-sn-glycero-3-phosphocholine (DOPC). In some embodiments, the lipid composition further comprises a polymer conjugated lipid. In some embodiments, the polymer conjugated lipid comprises a polyethylene glycol (PEG) conjugated lipid. In some embodiments, the polymer conjugated lipid comprises 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k). In some embodiments, the lipid composition further comprises a steroid, a helper lipid, and a polymer conjugated lipid. In some embodiments, the ionizable lipid is present in the lipid composition at a weight percentage from about 30% to about 90%. In some embodiments, the steroid is present in the lipid composition at a weight percentage from about 10% to about 40%. In some embodiments, the helper lipid is present in the lipid composition at a weight percentage from about 1% to about 20%. In some embodiments, the polymer conjugated lipid is present in the lipid composition at a weight percentage from about 1% to about 20%. In some embodiments, the weight ratio of the ionizable lipid/steroid/helper lipid/polymer conjugated lipid is about 16/4/1/1. In some embodiments, the weight ratio of the pharmaceutical agent/lipid composition is from about 1:200 to about 1:5. In some embodiments, the lipid composition further comprises a steroid and a helper lipid. In some embodiments, the ionizable lipid is present in the lipid composition at a weight percentage from about 30% to about 90%. In some embodiments, the helper lipid is present in the lipid composition at a weight percentage from about 5% to about 40%. In some embodiments, the steroid is present in the lipid composition at a weight percentage from about 5% to about 40%. In some embodiments, the weight ratio of the ionizable lipid/steroid/helper lipid is about 2/1/1. In some embodiments, the lipid composition further comprises a pharmaceutically acceptable carrier.

In some embodiments, the pharmaceutically acceptable carrier comprises a sugar, wherein the sugar comprises mannitol, sucrose, maltose, or trehalose. In some embodiments, the carrier is present in the composition at a weight percentage from about 5% to about 60%. In some embodiments, the ionizable lipid comprises at least two hydrophobic tails, wherein not all hydrophobic tails are identical. In some embodiments, the ionizable lipid comprises at least two hydrophobic tails, wherein two or more hydrophobic tails are identical. In some embodiments, the pharmaceutical composition further comprises a polynucleotide, an oligonucleotide, a polypeptide, an oligopeptide, a small molecule compound, or any combination thereof. In some embodiments, the small molecule compound comprises a drug for chemotherapy.

In some embodiments, the physical properties of the lipid composition are more stable compared to other lipid compositions. In some embodiments, the physical properties of the lipid composition are stable for at least 2 months, at least 3 months, at least 4 months, at least 5 months, or at least 6 months or more when stored at 4° C., −20° C., or −80° C. In some embodiments, the physical properties of the lipid composition are stable for at least 5 months when stored at 4° C., −20° C., or −80° C. In some embodiments, the physical properties of the lipid composition comprise size, polydispersity index (PDI), encapsulation efficacy (EE %), or pKa of the lipid composition.

In some aspects, the present disclosure provides a method for depleting B cells, comprising administering a pharmaceutical composition provided herein to a subject in need thereof, wherein the subject in need thereof has a disorder that requires a B cell depletion therapy (BCDT). In some embodiments, the disorder that requires a B cell depletion therapy (BCDT) comprises a B-cell malignancy or an autoimmune disorder. In some embodiments, the B-cell malignancy comprises B-cell lymphoma, wherein the B-cell lymphoma comprises diffuse large B-cell lymphoma (DLBCL), follicular lymphoma, chronic lymphocytic leukemia (CLL), small lymphocytic lymphoma (SLL), Mantle cell lymphoma (MCL), Marginal zone lymphomas, Burkitt lymphoma, Lymphoplasmacytic lymphoma (Waldenstrom macroglobulinemia), Hairy cell leukemia, primary central nervous system (CNS) lymphoma, or primary intraocular lymphoma (lymphoma of the eye). In some embodiments, the B-cell lymphoma affects the spleen or lymph nodes. In some embodiments, the B-cell malignancy comprises multiple myeloma. In some embodiments, the autoimmune disorder comprises allergic disorders, systemic lupus erythematosus (SLE), rheumatoid arthritis (RA), multiple sclerosis (MS), anti-myelin-oligodendrocyte glycoprotein (anti-MOG) spectrum disorders, Neuromyelitis Optica Spectrum Disorder (NMOSD), anti-NMDAR encephalitis, or myasthenia gravis. In some embodiments, the disorder that requires a B cell depletion therapy (BCDT) further comprises pemphigus vulgaris or Sjogren Syndrome.

In some aspects, the present disclosure provides a method for treating a blood malignancy, comprising administering a pharmaceutical composition provided herein to a subject in need thereof, wherein the subject in need thereof has a blood malignancy. In some embodiments, the blood malignancy comprises B-cell lymphoma, wherein the B-cell lymphoma comprises diffuse large B-cell lymphoma (DLBCL), follicular lymphoma, chronic lymphocytic leukemia (CLL), small lymphocytic lymphoma (SLL), Mantle cell lymphoma (MCL), Marginal zone lymphomas, or Burkitt lymphoma. In some embodiments, the administering comprises administering topically, orally, or by an injection. In some embodiments, the administering comprises administering via an intravenous injection or via an intramuscular injection. In some embodiments, the method results in the expression of a therapeutic peptide that has a longer half-life compared to any other method comprising administering an equivalent dose of the therapeutic peptide.

In some embodiments, the method results in the expression of a therapeutic peptide that has a half-life of at least 2 hours, at least 2.5 hours, at least 3 hours, at least 3.5 hours, at least 4 hours, at least 4.5 hours, at least 5 hours, at least 5.5 hours, at least 6 hours, at least 6.5 hours, at least 7 hours, at least 7.5 hours, at least 8 hours, at least 8.5 hours, at least 9 hours, at least 9.5 hours, at least 10 hours, at least 12 hours, at least 16 hours, at least 18 hours, at least 24 hours, at least 1.5 days, at least 2 days, at least 2.5 days, at least 3 days, at least 3.5 days, at least 4 days, at least 4.5 days, at least 5 days, at least 5.5 days, at least 6 days, at least 6.5 days, at least 7 days, at least 7.5 days, at least 8 days, at least 8.5 days, at least 9 days, at least 9.5 days, at least 10 days, at least 11 days, at least 12 days, at least 13 days, at least 14 days, or at least 15 or more days.

In some embodiments, the method results in the expression of a therapeutic peptide that has a larger area under curve (AUC) compared to other methods. In some embodiments, the method results in the expression of a therapeutic peptide that has an AUC that is at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, or 500% or more larger compared to any other method comprising administering an equivalent dose of the therapeutic peptide.

In some embodiments, the method results in prolonged B-cell depletion compared to any other method comprising administering an equivalent dose of the therapeutic peptide. In some embodiments, the prolonged B-cell depletion comprises B cell depletion for at least 6 hours, at least 12 hours, at least 18 hours, at least 24 hours, at least 3 days, at least 4 days, at least 5 days, at least 6 days, at least 7 days, at least 8 days, at least 9 days, at least 10 days, at least 15 days, at least 17.5 days, at least 20 days, at least 25 days, at least 27.5 days, at least 30 days, at least 35 days, at least 37.5 days, at least 40 days, at least 45 days, at least 47.5 days, at least 50 days, at least 55 days, at least 57.5 days, at least 60 days, at least 65 days, at least 70 days, at least 75 days, at least 80 days, at least 85 days, at least 90 days, at least 95 days, at least 100 days, at least 105 days, at least 110 days, or at least 120 or more days. In some embodiments, the method results in depletion of at least 50%, at least 55%, at least 60%, at least 65%, at least 70%, at least 75%, at least 80%, at least 85%, at least 90%, at least 95%, or at least 99% or more of circulating B-cells in a subject. In some embodiments, the method results in depletion of at least 50%, at least 55%, at least 60%, at least 65%, at least 70%, at least 75%, at least 80%, at least 85%, at least 90%, at least 95%, or at least 99% or more of tissue B-cells in a subject. In some embodiments, the method results in depletion of at least 50%, at least 55%, at least 60%, at least 65%, at least 70%, at least 75%, at least 80%, at least 85%, at least 90%, at least 95%, or at least 99% or more of naive B-cells in a subject. In some embodiments, the method results in depletion of at least 50%, at least 55%, at least 60%, at least 65%, at least 70%, at least 75%, at least 80%, at least 85%, at least 90%, at least 95%, or at least 99% or more of mature B-cells in a subject. In some embodiments, the mature B-cell comprises a memory B-cell, a plasma cell, or a plasmablast. In some embodiments, the method results in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200% or more B-cell depletion compared to any other method comprising administering an equivalent dose of the therapeutic peptide.

In some embodiments, the method further comprises administering a small molecule drug. In some embodiments, the small molecule drug comprises an agent for chemotherapy. In some embodiments, the subject in need thereof comprises a mammal.

In some embodiments, the mammal comprises a human, a non-human primate, or a rodent. In some embodiments, the pharmaceutical agent is administered in one or more doses. In some embodiments, the pharmaceutical agent is administered at a dosage of no more than 3 mg/kg (mg of nucleic acid per kg of body weight). In some embodiments, the pharmaceutical agent is administered at a dosage from about 0.007 mg/kg to about 0.2 mg/kg (mg of nucleic acid per kg of body weight).

In some embodiments, the pharmaceutical agent is administered at a dosage from about 0.014 mg/kg to about 0.1 mg/kg (mg of nucleic acid per kg of body weight).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates synthesis schemes of exemplary lipid tails.

FIGS. 2A-2C depict characterization and physicochemical properties of leading L88-mRNA compared with reference ALC-0315-mRNA. hEPO mRNA was used as the surrogate cargo to be formulated with indicated active lipids. As depicted in FIG. 2A, size and polydispersity index (PDI) were determined by Dynamic Light Scattering (DLS). FIG. 2B shows the % of Encapsulation efficiency (EE %), which were determined by RiboGreen assay. FIG. 2C shows the surface pKa values of LNPs determined by TNS assay. Data points are presented as means±SD.

FIGS. 3A-3C illustrate characterization and physicochemical properties of L88-003 in long-time stability study. The size, PD, EE % and mRNA integrity of L88-003 were detected at the timepoint of fresh preparation or stored at either 4° C. or −80° C. for 5 months.

FIGS. 4A-4B illustrate tissue distribution of L88 in WT mice. Firefly luciferase mRNA was used as the surrogate cargo to be formulated with L88 and i.v. injected into CD1 mice (n=2). FIG. 4A shows representative results from whole body and organ luciferase imaging performed by IVIS at 6 h post-injection. As depicted in FIG. 4B, luciferase protein in tissue lysis from indicated organs were quantified by luciferase activity assay. Data are presented as means±SD.

FIG. 5 depicts cellular tropism analysis of L88 in liver, spleen and bone marrow determined in Ai9 mouse model by flow cytometry. Cre recombinase mRNA were delivered by L88 formulation through I.V. administration into Ai9 mice (n=2). % of Tdtomato+ cells were calculated in indicated cell types by flow cytometry after 7-day treatment of each mouse. Data are presented as means±SD.

FIG. 6 depicts protein expression level of repeat dose L88-hEPO mRNA. 0.5 mg/kg of LNP comprising human erythropoietin (hEPO) mRNA was weekly i.v. injection into CD1 mice (n=5) for 5 weeks. Plasma hEPO level of each mouse were quantified by ELISA assay at 6 h after each dose, and each data point were presented as means±SD.

FIGS. 7A-7B show liver clearance analysis of single dose I.V. administration of L88 in CD1 mice. As depicted in FIG. 7A, lipid level of L88 in mouse liver (n=2 for each group) at 6 h after treated with indicated dosage of L88-Luciferase mRNA through I.V. injection were determined by LC-MS. In FIG. 7B, L88 and ALC-0315 lipid amount in liver were analysis at indicated timepoint after iv injection of 0.5 mg/kg LNP-Luc mRNA by LC-MS. The relative amount was normalized to individual tissue weight, and represent as means±SD.

FIG. 8A is a schematic of an mRNA molecule encoding CD19/CD3 BiTE. FIGS. 8B-8C show that mRNAs encoding CD19/CD3 BiTE encapsulated by a plurality of LNPs were delivered to cell culture. Soluble BiTE protein was expressed in all groups testes, and the BiTE protein in cell supernatant (FIG. 8B) and purified BiTE protein (FIG. 8C) bind to CD19 on ELISA plates. At least three mRNA designs were tested. HTX-01-001 mRNA comprises SEQ ID NOs: 1-4. HTX-01-002 mRNA comprises SEQ ID NOs: 5-8. HTX-01-003 mRNA comprises SEQ ID NOs: 7-10.

FIGS. 9A-9B show that the CD19/CD3 BiTE mediate T cell killing of cancer cells. As shown in both FACs analysis (FIG. 9A) and calculated result after normalized against no treatment control (FIG. 9B), CD19-CD3 BiTEs, which were produced by in vitro transfection of the mRNAs encoding the BiTE, can induce strong T cell killing activity against CD19 positive Raji cells.

FIG. 10A depicts size distribution of a plurality of LNPs comprising the mRNA encoding CD19/CD3 BiTE. FIG. 10B depicts encapsulation efficiency of the LNPs comprising the mRNA encoding CD19/CD3 BiTE.

FIGS. 11A-11B show the ex vivo efficacy of BiTE against CD19-expressing cancer cells. LNPs comprising mRNA encoding CD19/CD3 BiTE were injection intravenously to BALB/c mice. Plasma of treated mice were used in tumor killing assay.

FIG. 11A shows the ex vivo tumor killing activity of BiTE. FIG. 11B shows plasma concentration of BiTE quantified by ELISA. Both L88 and L93 lipid nanoparticles were used.

FIGS. 12A-12B show PK profiling of single dose LNP-CD19/CD3 BiTE mRNA in Balb/c mice. FIG. 12A is a schematic diagram of single dose PK study experimental design. As depicted in FIG. 12B, CD19-CD3 BiTE protein concentration in the plasma of Balb/c mice after i.v. administration of LNP-mRNA. Mice were treated with total 5 μg BiTE mRNA. 5 mice were blood draw of each timepoint for quantification by CD3 ELISA assay. Concentrations from technical ELISA duplicates are shown. Data are presented as means±SD.

FIGS. 13A-13H depict results from the efficacy study of L88-003 performed in hPBMC-reconstituted Raji-Luciferase xenograft mouse model. FIG. 13A is the schematic diagram of study design with tumor cell inoculation and regiment of 3 treatment groups (n=5 for each group) including LNP-mRNA (L88-003, 1.6 μg per mouse), recombinant BiTE protein (rPR003, 10 μg per mouse), and vehicle. FIG. 13B depicts the total photon flux images of individual mice in each group were captured at indicated timepoints by IVIS. As depicted in FIGS. 13C-13G, bioluminescence signal was quantitated as photons/see using living Image 4.7 software for each treatment group. Individual total flux (TF) at end-timepoint of this study were analysis. Each dot represent bioluminescence signal of individual mouse and statistical analysis were compared to G1_Vehicle control group by Kruskal-Wallis test with Dunn's multiple comparisons test. ***p<0.01. E,F,G) TF changes of each mouse in individual groups during the treatment were analysis. As depicted in FIG. 13H, relative body weight change of each mouse during the treatment were normalized to baseline timepoint (t=0) and each point represent as means±SD.

FIGS. 14A-14B show the plasma CD19-CD3 BiTE protein PK profiling for escalated dose of IV infusion of L88-003 in cynomolgus monkey. FIG. 14A is a schematic diagram of 3 escalation dose regiment and blood collection at indicated timepoints for PK profiling. FIG. 14B depicts CD19-CD3 BiTE protein concentration in the plasma of cynomolgus monkey at indicated timepoints after IV infusion of L88-003 quantified in a CD3 ELISA assay. Data are presented as means±SD of technical ELISA duplicates and dotted line depicts the LLOQ of 2 ng/ml.

FIGS. 15A-15H show circulating B-cell depletion and T-cell dynamic analysis in cynomolgus monkey following 3 weekly escalated doses of L88-003 (e.g., LNP-CD19 BiTE mRNA). FIG. 15A is a schematic diagram of 3 escalation dose regiment and blood collection at indicated timepoints for peripheral B and T-cell phenotyping by flow cytometry. FIG. 5Cshows that treatment with LNP-CD19 BiTE mRNA depleted circulating plasmablasts and plasma cells. FIG. 15B and FIG. 15D show the absolute cell count of circulating CD20+ B-cells and circulating CD3+ T cells respectively at indicated timepoints. FIG. 15E and FIG. 15F show the absolute cell count of circulating CD3+CD4+T helper cells and CD3+CD8+ CTLs at indicated timepoints respectively. FIG. 15G and FIG. 15H show the % of CD69+ activated T-cells in CD4 and CD8 T-cell subgroups respectively.

FIG. 16 depicts the relative body weight changes monitored twice per week and normalized to baseline timepoint (t=0).

FIGS. 17A-17I depict results from the non-clinical tolerability study in cynomolgus monkey following 3 weekly escalated doses of L88-003. FIG. 17A is a schematic diagram of 3 escalation dose regiment and blood collection at indicated timepoints for clinical hematology, clinical chemistry and cytokine analysis. FIGS. 17B-17D show whole blood CBC hematology analysis evaluated at indicated timepoints. Absolute counts of white blood cells (WBC), red blood cells (RBC), and platent (PLT) graphed respectively in FIG. 17B, FIG. 17C, and FIG. 17D. The dotted lines depict the reference range and/or pre-dose readouts. FIGS. 17E-17G show clinical chemistry analysis detected at indicated timepoints. Serum concentration of alanine aminotransferase (ALT), aspartate aminotransferase (AST), and urea nitrogen (BUN) graphed respectively in FIG. 17E, FIG. 17F, and FIG. 17G. The dotted lines depict the reference range and/or pre-dose readouts. FIG. 17 H and FIG. 17I show plasma cytokine levels including IL6, IL2, IL4, IFNγ, and TNFα levels quantified by multiplex luminex assay.

### DETAILED DESCRIPTION

Messenger RNA (mRNA) as a platform for efficient protein expression in vivo has many advantages over other therapeutic approaches. A large size and negative charges of mRNA are obstacles to it efficiently reaching the cytosol. Naked mRNA can be spontaneously taken up by different cell types but this usually results in degradation in acidic endolysosomal compartments. Lipid nanoparticles (LNPs) have been identified as an efficient way to protect mRNA from ubiquitous RNAses, shield it from immune cells and deliver it to cells while enabling escape from the endosome. LNPs generally consist of four major components: an ionizable, a sterol, a phospholipid and a lipid-anchored polyethylene glycol (PEG). The phospholipid and sterol work together to stabilize the LNP, the lipid-anchored PEG provides vial and storage stability and the ionizable lipid is critical for cellular uptake and endosomal escape, allowing for release of the mRNA into the cytosol. By changing the ratio and identities of the lipid components, the efficacy and tolerability of the formulation can be altered. Importantly, mRNA/LNP formulation requires careful process controls to ensure reproducibility of manufacturing and stability.

Although full-length antibodies have engendered significant success targeting cancers, immune disorders and infectious diseases, they are relatively large and exhibit less tissue penetrance than small molecules. Contrastingly, therapeutic peptides derived from fragments of antibodies, such as bispecific T-cell engagers (e.g., BiTE), can have a small size that enables better tissue penetration. However, the use of these molecules has been hampered by their short half-lives of several hours and propensity to aggregate. Both properties could be improved or avoided through mRNA-mediated expression since antibody will be continuously expressed from the mRNA until the mRNA is degraded. This approach would also prevent the aggregation often observed during protein purification. The LNP formulations that encapsulate mRNA for delivery can offer advantages for this technology over recombinant proteins as LNPs can offer greater biodistribution, with more tissue penetration than recombinant antibody proteins. LNPs optimized for mRNA delivery can result in higher expression of therapeutic peptides via more convenient routes of administration, such as subcutaneous or IM.

The present disclosure provides pharmaceutical compositions and methods related to lipid nanoparticles (LNPs) comprising ionizable lipids and nucleic acid sequences encoding therapeutic peptides. In some embodiments, the pharmaceutical composition comprises: a) a synthetic nucleic acid sequence encoding a therapeutic peptide, wherein the therapeutic peptide is configured to bind to a B-cell antigen; and b) a plurality of lipid nanoparticles; wherein the synthetic nucleic acid molecule is encapsulated within at least one of the plurality of lipid nanoparticles. The pharmaceutical compositions and methods provided herein can result in high levels of therapeutic peptide expression in a subject. In some embodiments, the pharmaceutical composition and methods result in drastic and prolonged B cell depletion due to expression of the therapeutic peptides in vivo. In some embodiments, the pharmaceutical composition and methods result in zero to minimum toxicity in a subject.

### Therapeutic Peptides

Some aspects of the present disclosure relate to a pharmaceutical composition comprising a synthetic nucleic acid molecule encoding a therapeutic peptide. The nucleic acid molecule can comprise nucleic acid sequences. The synthetic nucleic acid molecule or the nucleic acid sequence can be DNA. The synthetic nucleic acid molecule or the nucleic acid sequence can be RNA. In some embodiments, the synthetic nucleic acid molecule or the nucleic acid sequence comprises a messenger RNA (mRNA) encoding the therapeutic peptide. In some embodiments, the therapeutic peptide is configured to bind or is capable of binding an antigen on a B lymphocyte (e.g., a B cell or a B-cell). In some embodiments, the therapeutic peptide preferentially binds to a B cell antigen. In some embodiments, the therapeutic peptide is configured to bind a B cell antigen with higher affinity than other antigens. The capacity of the therapeutic peptide to bind any antigen can be measured by a dissociation constant (Kd). In some embodiments, the therapeutic peptide has a smaller Kd for a B cell antigen binding than for any other antigen. In some embodiments, the therapeutic peptide has a Kd for a B cell antigen in the nano molar or micro molar range.

The therapeutic peptide provided herein may be configured to bind or may be capable of binding to any B cell antigen. A B cell antigen can refer to any macromolecule expressed on the surface of a B cell. In some embodiments, the B cell antigen comprises a surface protein of the B cell. In some embodiments, the B cell antigen comprises a membrane-bound protein of the B cell. In some embodiments, the B cell antigen comprises a membrane-associated protein of the B cell. In some embodiments, the B cell antigen comprises a glycosylated protein of the B cell. In some embodiments, the B cell antigen comprises a peptide, a glycan, or a combination thereof.

The therapeutic peptide can be configured to bind or is capable of binding to a B cell antigen expressed on a B cell at any stage of cell development. In some embodiments, the B cell antigen can be expressed by any subtype of B cell. In some embodiments, the B-cell antigen comprises a memory B-cell antigen, a naïve B-cell antigen, a plasmablast antigen, or a plasma cell antigen. In some embodiments, the B-cell antigen is selected from the group consisting of CD10, CD19, CD20, CD22, CD27, CD32b, CD38, CD40, B-cell maturation antigen (BCMA), B-cell activating factor receptor (BAFFR), CD138, and CD5. In some embodiments, the B-cell antigen comprises CD10, CD19, CD20, CD22, CD27, CD32b, CD38, CD40, B-cell maturation antigen (BCMA), B-cell activating factor receptor (BAFFR), CD138, or CD5, or any combination thereof. In some embodiments, the B-cell antigen comprises CD19.

The therapeutic peptide provided herein can is configured to bind to one or more antigens. In some embodiments, the therapeutic peptide provided herein is configured to bind to two or more antigens. In some embodiments, the therapeutic peptide is configured to bind to two or more B cell antigens. In some embodiments, the therapeutic peptide is configured to bind to a first antigen (e.g., a B cell antigen) and a second antigen. In some embodiments, the second antigen is different from the first antigen (e.g., B cell antigen). In some embodiments, the second antigen is also a B cell antigen. In some embodiments, the second antigen is not a B cell antigen.

In some embodiments, the therapeutic peptide is configured to bind a second antigen, wherein the second antigen comprises an immune cell antigen or a tumor antigen. An immune cell antigen can refer to any macromolecule expressed on the surface of an immune cell. In some embodiments, the immune cell antigen comprises a surface protein of the immune cell. In some embodiments, the immune cell antigen comprises a membrane-bound protein of the immune cell. In some embodiments, the immune cell antigen comprises a membrane-associated protein of the immune cell. In some embodiments, the immune cell antigen comprises a glycosylated protein of the immune cell. In some embodiments, the immune cell antigen comprises a peptide, a glycan, or a combination thereof. A tumor cell antigen can refer to any macromolecule expressed on the surface of a tumor cell. In some embodiments, the tumor cell antigen comprises a surface protein of the tumor cell. In some embodiments, the tumor cell antigen comprises a membrane-bound protein of the tumor cell. In some embodiments, the tumor cell antigen comprises a membrane-associated protein of the tumor cell. In some embodiments, the tumor cell antigen comprises a glycosylated protein of the tumor cell.

In some embodiments, the tumor cell antigen comprises a peptide, a glycan, or a combination thereof. In some embodiments, the therapeutic peptide is capable of binding to an immune cell antigen, which comprises a T-cell antigen, a Treg cell antigen, or a natural killer cell (NK-cell) antigen. In some embodiments, the therapeutic peptide is capable of binding to CD3.

In some embodiments, the therapeutic peptide comprises an antibody or an antigen-binding fragment thereof. In some embodiments, the antibody or the antigen-binding fragment thereof comprises a bispecific antibody, a multispecific antibody, a chimeric antigen receptor (CAR), or an antigen-binding fragment thereof. In some embodiments, the antibody or the antigen-binding fragment may comprise a light chain or a heavy chain. In some embodiments, the antigen-binding fragment can comprise a fragment antigen-binding (Fab or F(ab)) domain, or a variant thereof, for example, F(ab′) or F(ab′)2. In some embodiments, the antigen-binding fragment can comprise Fab, fragment variable region (Fv), recombinant immuno-globulins (rIgG), single-chain variable fragments (scFv), heavy chain antibodies (hcAbs), a single domain antibody, Variable Heavy domain of Heavy chain (VHH), variable domain of new antigen receptor (VNAR), single-domain antibody (sdAbs), or nanobody. In some embodiments, the antigen-binding fragment thereof comprises a scFv. In some embodiments, the antibody or antigen-binding fragment thereof comprises a bispecific antibody or a multispecific antibody.

In some embodiments, the therapeutic peptide comprises a bispecific antibody or the antigen-binding fragment thereof. In some embodiments, the therapeutic peptide comprises a scFv bispecific antibody or antigen binding fragment. In some embodiments, the therapeutic peptide comprises a bispecific T-cell engager (BiTE). In some embodiments, the therapeutic peptide comprises a BiTE that binds to CD19 and CD 3.

In some embodiments, the therapeutic peptide comprises an FDA-approved drug that is configured to bind to a B-cell antigen. In some embodiments, the FDA-approved drug comprises blinatumomab, inebilizumab, loncastuximab, or tafasitamab.

In some embodiments, the therapeutic peptide can be operably linked to another peptide. In some embodiments, the therapeutic peptide can be produced by a cell from another peptide. In some embodiments, the therapeutic peptide can be produced by a cell from a peptide comprising a signal peptide. The signal peptide can be cleaved in the cell and therefore a therapeutic peptide is produced. In some embodiments, the therapeutic peptide can be operably linked to a protein tag. In some embodiments, the protein tag comprises a histidine tag, a flag tag or a hemagglutinin tag. In some embodiments, the protein tag is selected from a group consisting of: a CBP tag, a flag tag, a GST tag, an HA tag, an HBH tag, an MBP tag, a myc tag, a his tag, an S tag, a SUMO tag, a TAP tab, a TRX tag, an E tag, an E2 tag, a KT3, a T7, a VSVG, an OLLAS, a Protein C, an NE tag, an Xpress tag, an Avi, and a V5 tag, and any combinations thereof. In some embodiments, the protein tag comprises a CBP tag, a flag tag, a GST tag, an HA tag, an HBH tag, an MBP tag, a myc tag, a his tag, an S tag, a SUMO tag, a TAP tab, a TRX tag, an E tag, an E2 tag, a KT3, a T7, a VSVG, an OLLAS, a Protein C, an NE tag, an Xpress tag, an Avi, or a V5 tag, or a portion of any one of these, or any combinations thereof.

In some embodiments, the therapeutic peptide comprises a heavy chain and a light chain. In some embodiments, the therapeutic peptide comprises an anti-CD19 heavy chain and an anti-CD19 light chain. In some embodiments, the therapeutic peptide further comprises an anti-CD3 heavy chain and an anti-CD3 light chain. In some embodiments, the anti-CD19 heavy chain has the amino acid sequence of SEQ ID NO: 30, 34, or 38. In some embodiments, the anti-CD19 light chain has the amino acid sequence of SEQ ID NO: 29, 33, or 37. In some embodiments, the anti-CD3 heavy chain has the amino acid sequence of SEQ ID NO: 32 or 36. In some embodiments, the anti-CD3 light chain has the amino acid sequence of SEQ ID NO: 31 or 35.

In some embodiments, the anti-CD19 light chain is encoded by the nucleic acid sequence of SEQ ID NOs: 1, 3, 5, or 9. In some embodiments, the anti-CD19 heavy chain is encoded by the nucleic acid sequence of SEQ ID NOs: 2, 4, 6, or 10. In some embodiments, the anti-CD19 light chain is encoded by nucleic acid sequences having 90% sequence identity to SEQ ID NOs: 1, 3, 5, or 9. In some embodiments, the anti-CD19 heavy chain is encoded by the nucleic acid sequences having 90% sequence identity to SEQ ID NOs: 2, 4, 6, or 10.

In some embodiments, the anti-CD3 light chain is encoded by the nucleic acid sequence of SEQ ID NO: 7. In some embodiments, the anti-CD3 light chain is encoded by nucleic acid sequences having 90% sequence identity to SEQ ID NO: 7. In some embodiments, the anti-CD3 heavy chain is encoded by the nucleic acid sequence of SEQ ID NO: 8. In some embodiments, the anti-CD3 heavy chain coding region comprises nucleic acid sequences having 90% sequence identity to SEQ ID NO: 8.

In some embodiments, the anti-CD19 light chain comprises SEQ ID NO: 29 and the anti-CD19 heavy chain comprises SEQ ID NO: 30; wherein the anti-CD3 light chain comprises SEQ ID NO: 31 and the anti-CD3 heavy chain comprises SEQ ID NO: 32.

In some embodiments, the anti-CD19 light chain comprises SEQ ID NO: 33 and the anti-CD19 heavy chain comprises SEQ ID NO: 34; wherein the anti-CD3 light chain comprises SEQ ID NO: 35 and the anti-CD3 heavy chain comprises SEQ ID NO: 36.

In some embodiments, the anti-CD19 light chain comprises SEQ ID NO: 37 and the anti-CD19 heavy chain comprises SEQ ID NO: 38; wherein the anti-CD3 light chain comprises SEQ ID NO: 35 and the anti-CD3 heavy chain comprises SEQ ID NO: 36.

In some embodiments, the anti-CD19 heavy chain consists of or comprises a light chain of blinatumomab, inebilizumab, loncastuximab, or tafasitamab. In some embodiments, the anti-CD19 heavy chain consists of or comprises a heavy chain of blinatumomab, inebilizumab, loncastuximab, or tafasitamab.

In some embodiments, after the pharmaceutical composition is administered to the subject, the therapeutic peptide encoded by the synthetic nucleic acid sequence of the pharmaceutical composition results in activation of CD69+ T cells.

The therapeutic peptide encoded by the nucleic acid sequences (e.g., mRNA) of the compositions provided herein can have superior pharmacokinetic profiles than corresponding recombinant therapeutic peptides binding the same antigens. After the pharmaceutical composition is administered to the subject, the therapeutic peptide encoded by the nucleic acid sequence can be expressed by the subject in vivo. The resulted therapeutic peptide may have superior potency and/or efficacy than recombinant therapeutic peptides binding the same antigen. In some embodiments, after the pharmaceutical composition is administered to a subject, the resulted therapeutic peptide has a longer half-life compared to a corresponding therapeutic peptide of another pharmaceutical composition. In some embodiments, after the pharmaceutical composition is administered to a subject, the therapeutic peptide expressed by the subject has a half-life of at least 2 hours, at least 2.5 hours, at least 3 hours, at least 3.5 hours, at least 4 hours, at least 4.5 hours, at least 5 hours, at least 5.5 hours, at least 6 hours, at least 6.5 hours, at least 7 hours, at least 7.5 hours, at least 8 hours, at least 8.5 hours, at least 9 hours, at least 9.5 hours, at least 10 hours, at least 12 hours, at least 16 hours, at least 18 hours, at least 24 hours, at least 1.5 days, at least 2 days, at least 2.5 days, at least 3 days, at least 3.5 days, at least 4 days, at least 4.5 days, at least 5 days, at least 5.5 days, at least 6 days, at least 6.5 days, at least 7 days, at least 7.5 days, at least 8 days, at least 8.5 days, at least 9 days, at least 9.5 days, at least 10 days, at least 11 days, at least 12 days, at least 13 days, at least 14 days, or at least 15 or more days.

In some embodiments, after the pharmaceutical composition is administered to a subject, the therapeutic peptide expressed by the subject has a larger area under curve (AUC) compared to an equivalent dose of a corresponding therapeutic peptide of another pharmaceutical composition.

In some embodiments, after the pharmaceutical composition is administered to a subject, the therapeutic peptide expressed by the subject has an AUC that is at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, or 500% or more larger compared to an equivalent dose of a corresponding therapeutic peptide of another pharmaceutical composition.

In some embodiments, after the pharmaceutical composition is administered to a subject, the therapeutic peptide expressed by the subject results in prolonged B-cell depletion compared to an equivalent dose of a corresponding therapeutic peptide of another pharmaceutical composition.

The therapeutic peptide encoded by the nucleic acid sequences (e.g., mRNA) of the compositions provided herein can have superior pharmacodynamic profiles than corresponding recombinant therapeutic peptides binding the same antigens. The therapeutic peptide encoded by the nucleic acid sequence of the compositions can result in prolonged and/or more drastic B cell depletion in vivo, after the pharmaceutical composition is administered to a subject. In some embodiments, the prolonged B-cell depletion comprises B cell depletion for at least 6 hours, at least 12 hours, at least 18 hours, at least 24 hours, at least 3 days, at least 4 days, at least 5 days, at least 6 days, at least 7 days, at least 8 days, at least 9 days, at least 10 days, at least 15 days, at least 17.5 days, at least 20 days, at least 25 days, at least 27.5 days, at least 30 days, at least 35 days, at least 37.5 days, at least 40 days, at least 45 days, at least 47.5 days, at least 50 days, at least 55 days, at least 57.5 days, at least 60 days, at least 65 days, at least 70 days, at least 75 days, at least 80 days, at least 85 days, at least 90 days, at least 95 days, at least 100 days, at least 105 days, at least 110 days, or at least 120 or more days.

In some embodiments, upon administration to the subject, the therapeutic peptide expressed by the subject results in depletion of at least 50%, at least 55%, at least 60%, at least 65%, at least 70%, at least 75%, at least 80%, at least 85%, at least 90%, at least 95%, or at least 99% or more of circulating B-cells in a subject. In some embodiments, after the pharmaceutical composition is administered to a subject, the therapeutic peptide expressed by the subject results in depletion of at least 50%, at least 55%, at least 60%, at least 65%, at least 70%, at least 75%, at least 80%, at least 85%, at least 90%, at least 95%, or at least 99% or more of tissue B-cells in a subject.

In some embodiments, upon administration to the subject, the therapeutic peptide expressed by the subject results in depletion of at least 50%, at least 55%, at least 60%, at least 65%, at least 70%, at least 75%, at least 80%, at least 85%, at least 90%, at least 95%, or at least 99% or more of naive B-cells in a subject. In some embodiments, after the pharmaceutical composition is administered to a subject, the therapeutic peptide expressed by the subject results in depletion of at least 50%, at least 55%, at least 60%, at least 65%, at least 70%, at least 75%, at least 80%, at least 85%, at least 90%, at least 95%, or at least 99% or more of mature B-cells in a subject.

In some embodiments, upon administration to the subject, the therapeutic peptide expressed by the subject results in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200% or more B-cell depletion compared to an equivalent dose of a corresponding therapeutic peptide of another pharmaceutical composition.

In some embodiments, after the pharmaceutical composition is administered to a subject, the therapeutic peptide expressed by the subject has zero to minimum toxicity. In some embodiments, the toxicity comprises transient elevation in cytokines or liver enzymes. In some embodiments, the toxicity comprises mild inflammation or mild hepatotoxicity.

### Nucleic Acids

The nucleic acid molecules and nucleic acid sequences provided herein can comprise ribonucleic acid (RNA) molecules and RNA sequences. The nucleic acid molecules and nucleic acid sequences provided herein can comprise deoxyribonucleic acid (DNA) molecules and DNA sequences. In some embodiments, the nucleic acids provided herein comprises a RNA, a DNA, a DNA/RNA hybrid, a nucleic acid analog, a chemically modified nucleic acid, a chimera composed of two or more nucleic acids or nucleic acid analogs, or any combination thereof. In some embodiments, the nucleic acid molecule and/or nucleic acid sequence is selected from the group consisting of a RNA, a DNA, a DNA/RNA hybrid, a nucleic acid analog, a chemically modified nucleic acid, a chimera composed of two or more nucleic acids or nucleic acid analogs, and any combination thereof.

The pharmaceutical composition provided herein comprises synthetic nucleic acid molecules (e.g., mRNA) encoding a therapeutic peptide. In some cases, the mRNA encapsulated by the lipid composition provided herein further comprises regulatory sequences that can facilitate and/or promote expression of the therapeutic peptide. The regulatory sequences can comprise a 5′ cap, a 5′ untranslated region, a promoter, a signal peptide sequence, a 3′ untranslated region, and a poly-A tail. In some cases, the regulatory sequences comprise an enhancer (e.g., CMV enhancer) to further enhance expression of the therapeutic peptide.

In some embodiments, the pharmaceutical composition comprises a synthetic nucleic acid sequence that further comprises a regulatory nucleic acid sequence. In some embodiments, the regulatory nucleic acid sequence comprises a promoter or a signal peptide. In some embodiments, the promoter comprises a tissue-specific promoter. In some embodiments, the synthetic nucleic acid sequence comprises a synthetic ribonucleic acid sequence (RNA). In some embodiments, the synthetic RNA comprises chemically modified nucleic acids, wherein the chemically modified nucleic acids improves the stability of the synthetic RNA. In some embodiments, the synthetic RNA comprises N6-methyladenosine (m6A), N6,2′-O-dimethyladenosine (m6Am), 8-oxo-7,8-dihydroguanosine (8-oxoG), pseudouridine (RA), 5-methylcytidine (m5C), or N4-acetylcytidine (ac4C), or any combinations thereof. In some embodiments, the synthetic RNA comprises a 5′ cap.

In some embodiments, the synthetic RNA comprises, in a 5′ to 3′ direction: a) a 5′ untranslated region (5′ UTR) coding region; b) a signal peptide coding region; c) a therapeutic peptide coding region; d) a 3′ untranslated region (3′ UTR) coding region; and e) a poly A tail coding region. TABLE 1. lists the nucleic acid sequences (RNA or DNA sequences) encoding CD19-CD3 BiTE and amino acid sequences of CD19-CD3 BiTE.

TABLE 1
Nucleic acid sequences and amino acid sequences.

| | SEQ | |
|---|---|---|
| | ID | |
| Name | NO: | Nucleic Acid Sequences (RNA or DNA sequences) |
| HD37 VL | 1 | GACAUUCAGCUGACCCAAAGCCCCGCCUCUCUGGCUGUGAG |
| (anti- | | CCUGGGACAAAGAGCCACCAUAAGCUGCAAAGCCAGCCAA |
| CD19) | | AGCGUUGACUAUGACGGAGACAGCUACCUGAACUGGUAUC |
| | | AGCAAAUCCCCGGACAGCCCCCCAAGCUGCUGAUCUACGAC |
| | | GCCAGCAACCUGGUGAGCGGCAUCCCCCCCCGAUUCAGCGG |
| | | AAGCGGCAGCGGAACCGACUUCACCCUCAACAUUCACCCCG |
| | | UGGAGAAGGUCGACGCCGCCACCUACCACUGCCAGCAGAGC |
| | | ACCGAAGAUCCCUGGACCUUCGGCGGCGGCACCAAGCUGGA |
| | | AAUCAAA |
| HD37 VH | 2 | CAGGUCCAACUCCAACAAAGCGGCGCCGAGCUCGUCAGACC |
| (anti- | | CGGCUCAAGCGUCAAAAUCAGCUGCAAAGCCAGCGGCUAC |
| CD19) | | GCCUUCAGCAGCUACUGGAUGAACUGGGUGAAGCAGAGAC |
| | | CCGGCCAAGGCCUCGAGUGGAUUGGACAAAUCUGGCCCGG |
| | | AGACGGAGACACCAACUACAACGGAAAAUUCAAAGGAAAA |
| | | GCCACACUGACCGCCGACGAAAGCAGUAGCACCGCCUACAU |
| | | GCAGCUGUCCAGCCUCGCCAGCGAAGACAGCGCCGUGUACU |
| | | UCUGCGCCAGACGGGAAACAACCACCGUGGGAAGAUACUA |
| | | CUACGCCAUGGACUACUGGGGCCAGGGAACCACCGUGACCG |
| | | UGAGCAGC |
| L2K07 | 3 | GACAUUCAGCUGACCCAAAGCCCCGCCAUAAUGAGCGCCA |
| VL (anti- | | GCCCCGGAGAAAAAGUGACCAUGACCUGUAGAGCCAGCAG |
| CD3) | | CAGCGUGAGCUAUAUGAACUGGUAUCAGCAAAAAAGCGG |
| | | CACCAGCCCCAAGAGAUGGAUCUACGACACCAGCAAGGUG |
| | | GCCAGCGGAGUGCCCUACAGAUUCUCCGGAAGCGGCAGCG |
| | | GAACAUCAUACAGCCUGACCAUCAGCAGCAUGGAAGCAGA |
| | | AGACGCCGCCACCUACUACUGCCAGCAGUGGAGCUCUAAC |
| | | CCCCUGACCUUCGGCGCCGGUACCAAGCUGGAGCUGAAA |
| L2K07 | 4 | GACAUUAAGCUGCAGCAGAGCGGCGCCGAGCUGGCCAGAC |
| VH (anti- | | CUGGAGCUUCCGUGAAAAUGAGCUGCAAAACCAGCGGCUA |
| CD3) | | CACCUUUACAAGAUAUACCAUGCACUGGGUGAAACAGAGA |
| | | CCCGGCCAAGGCCUGGAGUGGAUCGGGUACAUUAACCCCA |
| | | GCAGAGGCUACACCAACUACAACCAAAAAUUCAAGGACAA |
| | | GGCCACCCUGACCACCGACAAAAGCAGCAGCACCGCCUAC |
| | | AUGCAGCUGAGCAGCCUGACCAGCGAAGACAGCGCCGUGU |
| | | ACUACUGCGCCCGAUACUACGACGACCACUACUGCCUGGA |
| | | CUACUGGGGCCAAGGCACAACACUGACCGUGUCCAGC |
| FMC43 | 5 | GACAUUCAGAUGACCCAGACCACCAGCAGCCUCAGCGCCA |
| VL (anti- | | GCCUGGGCGACAGAGUUACCAUCUCCUGCAGAGCCAGCCA |
| CD19) | | AGACAUAAGCAAGUAUCUCAACUGGUAUCAGCAGAAACCC |
| | | GACGGCACCGUGAAGCUGCUCAUCUACCACACCAGCAGAC |
| | | UCCACAGCGGCGUUCCCAGCCGGUUCAGCGGAAGCGGCUC |
| | | CGGAACAGACUACUCCCUGACCAUCAGCAACCUCGAACAA |
| | | GAAGACAUCGCAACCUACUUCUGCCAGCAAGGCAACACAC |
| | | UCCCCUACACCUUCGGAGGCGGCACAAAGCUGGAGAUCAC |
| | | C |
| FMC43 | 6 | GAGGUGAAGCUGCAGGAGAGCGGGCCAGGACUGGUGGCCC |
| VH (anti- | | CUAGCCAGAGCCUGAGUGUGACAUGUACAGUGAGCGGAG |
| CD19) | | UGAGCCUGCCCGACUACGGAGUGAGCUGGAUUAGACAGCC |
| | | ACCUAGAAAAGGACUGGAGUGGCUGGGAGUGAUUUGGGG |
| | | CAGCGAGACCACAUACUACAACUCUGCCCUGAAGAGCAGG |
| | | CUGACCAUUAUAAAGGACAAUUCAAAGAGCCAGGUGUUCC |
| | | UGAAGAUGAAUAGCCUGCAGACCGAUGAUACCGCUAUCUA |
| | | CUACUGCGCCAAGCACUACUACUAUGGCGGAAGCUACGCC |
| | | AUGGACUACUGGGGACAGGGCACCAGCGUCACCGUGAGCA |
| | | GC |
| hSP34 VL | 7 | CAGACCGUCGUGACCCAAGAGCCCAGCCUCACCGUGAGCC |
| (anti- | | CCGGCGGAACAGUGACCCUCACCUGCGGAAGCAGCACAGG |
| CD3) | | AGCCGUGACCAGCGGAUACUACCCCAACUGGGUCCAACAG |
| | | AAACCCGGCCAAGCCCCCAGAGGCCUGAUCGGAGGAACCA |
| | | AGUUUCUCGCCCCCGGCACACCCGCAAGAUUCAGCGGAAG |
| | | CCUGCUCGGAGGCAAGGCCGCCCUGACCCUGAGCGGAGUG |
| | | CAACCUGAAGACGAGGCAGAGUACUACUGCGCUCUGUGGU |
| | | ACUCCAACAGAUGGGUGUUCGGAGGAGGCACAAAGCUGAC |
| | | CGUCCUG |
| hSP34 VH | 8 | GAGGUGCAGCUGGUGGAGAGCGGGGGGGGACUGGUGCAG |
| (anti- | | CCAGGAGGAAGCCUGAAGCUGAGCUGUGCCGCAAGCGGGU |
| CD3) | | UCACAUUUAAUAAGUACGCAAUGAACUGGGUGAGACAGG |
| | | CUCCUGGCAAAGGACUGGAGUGGGUGGCUAGAAUUAGAA |
| | | GCAAGUACAACAACUACGCCACAUACUAUGCCGACAGUGU |
| | | GAAGGACCGGUUCACCAUAAGCAGAGACGAUAGCAAGAA |
| | | UACCGCUUACCUGCAAAUGAACAACCUGAAGACCGAGGAC |
| | | ACCGCCGUCUACUACUGUGUGAGGCACGGAAACUUCGGCA |
| | | ACAGCUACAUCUCCUACUGGGCCUACUGGGGACAAGGGAC |
| | | CCUGGUGACCGUCAGCAGC |
| BU12 VL | 9 | GAGAAUGUGCUGACCCAGAGCCCCGCCAUAAUGAGCGCCA |
| (anti- | | GCCCAGGAGAGAAGGUGACAAUGACAUGCAGCGCAAGCAG |
| CD19) | | CAGCGUGAGUUACAUGCACUGGUACCAGCAGAAGAGCAGC |
| | | ACCAGCCCAAAGCUCUGGAUCUACGACACCAGCAAACUGG |
| | | CCAGUGGAGUGCCCGGCAGAUUCAGUGGAAGUGGAAGCG |
| | | GAAACAGCCACUUCCUGACUAUCAGCAGCAUGGAAGCCGA |
| | | AGACGUCGCCACCUACUACUGCUUUCAGGGAAGCGUGUAC |
| | | CCCUUCACCUUCGGAAGCGGUACCAAGCUGGAGAUCAAG |
| BU12 VH | 10 | CAGGUCACCCUCAAAGAGUCAGGCCCCGGCAUCCUCCAAC |
| (anti- | | CCAGCCAAACCCUCAGCCUCACAUGCAGCUUCAGCGGCUU |
| CD19) | | CAGCCUCUCCACCAGCGGCAUGGGCGUCGGCUGGAUCAGA |
| | | CAGCCCUCCGGAAAAGGCCUGGAAUGGCUCGCCCACAUCU |
| | | GGUGGGACGACGACAAAAGAUACAACCCCGCCCUGAAGUC |
| | | CCGGCUCACCAUCAGUAAAGACACCAGCUCCAACCAAGUC |
| | | UUCCUCAAGAUCGCCUCUGUGGACACCGCCGACACCGCCG |
| | | CUUAUUACUGCGCCAGAAUGGAACUCUGGUCCUACUACUU |
| | | CGACUACUGGGGACAAGGCACCACCCUCACCGUCUCCUCC |
| Signal | 11 | AAACUACAAGACAGACUUGCAAAAGAAGGCAUGCACAGCU |
| Peptide | | CAGCACUGCUCUGUUGCCUGGUCCUCCUGACUGGGGUGAG |
| | | GGCC |
| Linker-1 | 12 | GGUGGCGGAGGGUCUGGUGGUGGAGGAUCAGGGGGUGGA |
| | | GGUUCAGGAGGCGGGGGAAGU |
| Linker-2 | 13 | GGUGGCGGUGGAUCU |
| His-tag | 14 | CACCAUCACCAUCAUCAC |
| HD37 VL | 15 | GACATTCAGCTGACCCAAAGCCCCGCCTCTCTGGCTGTGAG |
| | | CCTGGGACAAAGAGCCACCATAAGCTGCAAAGCCAGCCAA |
| | | AGCGTTGACTATGACGGAGACAGCTACCTGAACTGGTATCA |
| | | GCAAATCCCCGGACAGCCCCCCAAGCTGCTGATCTACGACG |
| | | CCAGCAACCTGGTGAGCGGCATCCCCCCCCGATTCAGCGGA |
| | | AGCGGCAGCGGAACCGACTTCACCCTCAACATTCACCCCGT |
| | | GGAGAAGGTCGACGCCGCCACCTACCACTGCCAGCAGAGC |
| | | ACCGAAGATCCCTGGACCTTCGGCGGCGGCACCAAGCTGGA |
| | | AATCAAA |
| HD37 VH | 16 | CAGGTCCAACTCCAACAAAGCGGCGCCGAGCTCGTCAGACC |
| | | CGGCTCAAGCGTCAAAATCAGCTGCAAAGCCAGCGGCTACG |
| | | CCTTCAGCAGCTACTGGATGAACTGGGTGAAGCAGAGACCC |
| | | GGCCAAGGCCTCGAGTGGATTGGACAAATCTGGCCCGGAGA |
| | | CGGAGACACCAACTACAACGGAAAATTCAAAGGAAAAGCC |
| | | ACACTGACCGCCGACGAAAGCAGTAGCACCGCCTACATGCA |
| | | GCTGTCCAGCCTCGCCAGCGAAGACAGCGCCGTGTACTTCT |
| | | GCGCCAGACGGGAAACAACCACCGTGGGAAGATACTACTA |
| | | CGCCATGGACTACTGGGGCCAGGGAACCACCGTGACCGTGA |
| | | GCAGC |
| L2K07 | 17 | GACATTCAGCTGACCCAAAGCCCCGCCATAATGAGCGCCAG |
| VL | | CCCCGGAGAAAAAGTGACCATGACCTGTAGAGCCAGCAGC |
| | | AGCGTGAGCTATATGAACTGGTATCAGCAAAAAAGCGGCAC |
| | | CAGCCCCAAGAGATGGATCTACGACACCAGCAAGGTGGCC |
| | | AGCGGAGTGCCCTACAGATTCTCCGGAAGCGGCAGCGGAAC |
| | | ATCATACAGCCTGACCATCAGCAGCATGGAAGCAGAAGAC |
| | | GCCGCCACCTACTACTGCCAGCAGTGGAGCTCTAACCCCCT |
| | | GACCTTCGGCGCCGGTACCAAGCTGGAGCTGAAA |
| L2K07 | 18 | GACATTAAGCTGCAGCAGAGCGGCGCCGAGCTGGCCAGAC |
| VH | | CTGGAGCTTCCGTGAAAATGAGCTGCAAAACCAGCGGCTAC |
| | | ACCTTTACAAGATATACCATGCACTGGGTGAAACAGAGACC |
| | | CGGCCAAGGCCTGGAGTGGATCGGGTACATTAACCCCAGCA |
| | | GAGGCTACACCAACTACAACCAAAAATTCAAGGACAAGGC |
| | | CACCCTGACCACCGACAAAAGCAGCAGCACCGCCTACATGC |
| | | AGCTGAGCAGCCTGACCAGCGAAGACAGCGCCGTGTACTAC |
| | | TGCGCCCGATACTACGACGACCACTACTGCCTGGACTACTG |
| | | GGGCCAAGGCACAACACTGACCGTGTCCAGC |
| FMC43 | 19 | GACATTCAGATGACCCAGACCACCAGCAGCCTCAGCGCCAG |
| VL | | CCTGGGCGACAGAGTTACCATCTCCTGCAGAGCCAGCCAAG |
| | | ACATAAGCAAGTATCTCAACTGGTATCAGCAGAAACCCGAC |
| | | GGCACCGTGAAGCTGCTCATCTACCACACCAGCAGACTCCA |
| | | CAGCGGCGTTCCCAGCCGGTTCAGCGGAAGCGGCTCCGGAA |
| | | CAGACTACTCCCTGACCATCAGCAACCTCGAACAAGAAGAC |
| | | ATCGCAACCTACTTCTGCCAGCAAGGCAACACACTCCCCTA |
| | | CACCTTCGGAGGCGGCACAAAGCTGGAGATCACC |
| FMC43 | 20 | GAGGTGAAGCTGCAGGAGAGCGGGCCAGGACTGGTGGCCC |
| VH | | CTAGCCAGAGCCTGAGTGTGACATGTACAGTGAGCGGAGTG |
| | | AGCCTGCCCGACTACGGAGTGAGCTGGATTAGACAGCCACC |
| | | TAGAAAAGGACTGGAGTGGCTGGGAGTGATTTGGGGCAGC |
| | | GAGACCACATACTACAACTCTGCCCTGAAGAGCAGGCTGAC |
| | | CATTATAAAGGACAATTCAAAGAGCCAGGTGTTCCTGAAGA |
| | | TGAATAGCCTGCAGACCGATGATACCGCTATCTACTACTGC |
| | | GCCAAGCACTACTACTATGGCGGAAGCTACGCCATGGACTA |
| | | CTGGGGACAGGGCACCAGCGTCACCGTGAGCAGC |
| hSP34 VL | 21 | CAGACCGTCGTGACCCAAGAGCCCAGCCTCACCGTGAGCCC |
| | | CGGCGGAACAGTGACCCTCACCTGCGGAAGCAGCACAGGA |
| | | GCCGTGACCAGCGGATACTACCCCAACTGGGTCCAACAGAA |
| | | ACCCGGCCAAGCCCCCAGAGGCCTGATCGGAGGAACCAAG |
| | | TTTCTCGCCCCCGGCACACCCGCAAGATTCAGCGGAAGCCT |
| | | GCTCGGAGGCAAGGCCGCCCTGACCCTGAGCGGAGTGCAAC |
| | | CTGAAGACGAGGCAGAGTACTACTGCGCTCTGTGGTACTCC |
| | | AACAGATGGGTGTTCGGAGGAGGCACAAAGCTGACCGTCCT |
| | | G |
| hSP34 VH | 22 | GAGGTGCAGCTGGTGGAGAGCGGGGGGGGACTGGTGCAGC |
| | | CAGGAGGAAGCCTGAAGCTGAGCTGTGCCGCAAGCGGGTTC |
| | | ACATTTAATAAGTACGCAATGAACTGGGTGAGACAGGCTCC |
| | | TGGCAAAGGACTGGAGTGGGTGGCTAGAATTAGAAGCAAG |
| | | TACAACAACTACGCCACATACTATGCCGACAGTGTGAAGGA |
| | | CCGGTTCACCATAAGCAGAGACGATAGCAAGAATACCGCTT |
| | | ACCTGCAAATGAACAACCTGAAGACCGAGGACACCGCCGTC |
| | | TACTACTGTGTGAGGCACGGAAACTTCGGCAACAGCTACAT |
| | | CTCCTACTGGGCCTACTGGGGACAAGGGACCCTGGTGACCG |
| | | TCAGCAGC |
| BU12 VL | 23 | GAGAATGTGCTGACCCAGAGCCCCGCCATAATGAGCGCCAG |
| | | CCCAGGAGAGAAGGTGACAATGACATGCAGCGCAAGCAGC |
| | | AGCGTGAGTTACATGCACTGGTACCAGCAGAAGAGCAGCAC |
| | | CAGCCCAAAGCTCTGGATCTACGACACCAGCAAACTGGCCA |
| | | GTGGAGTGCCCGGCAGATTCAGTGGAAGTGGAAGCGGAAA |
| | | CAGCCACTTCCTGACTATCAGCAGCATGGAAGCCGAAGACG |
| | | TCGCCACCTACTACTGCTTTCAGGGAAGCGTGTACCCCTTCA |
| | | CCTTCGGAAGCGGTACCAAGCTGGAGATCAAG |
| BU12 VH | 24 | CAGGTCACCCTCAAAGAGTCAGGCCCCGGCATCCTCCAACC |
| | | CAGCCAAACCCTCAGCCTCACATGCAGCTTCAGCGGCTTCA |
| | | GCCTCTCCACCAGCGGCATGGGCGTCGGCTGGATCAGACAG |
| | | CCCTCCGGAAAAGGCCTGGAATGGCTCGCCCACATCTGGTG |
| | | GGACGACGACAAAAGATACAACCCCGCCCTGAAGTCCCGG |
| | | CTCACCATCAGTAAAGACACCAGCTCCAACCAAGTCTTCCT |
| | | CAAGATCGCCTCTGTGGACACCGCCGACACCGCCGCTTATT |
| | | ACTGCGCCAGAATGGAACTCTGGTCCTACTACTTCGACTACT |
| | | GGGGACAAGGCACCACCCTCACCGTCTCCTCC |
| Signal | 25 | AAACTACAAGACAGACTTGCAAAAGAAGGCATGCACAGCT |
| Peptide | | CAGCACTGCTCTGTTGCCTGGTCCTCCTGACTGGGGTGAGG |
| | | GCC |
| Linker-1 | 26 | GGTGGCGGAGGGTCTGGTGGTGGAGGATCAGGGGGTGGAG |
| | | GTTCAGGAGGCGGGGGAAGT |
| Linker-2 | 27 | GGTGGCGGTGGATCT |
| His-tag | 28 | CACCATCACCATCATCAC |

Amino acid sequences

| | | |
|---|---|---|
| HD37 VL | 29 | DIQLTQSPASLAVSLGQRATISCKASQSVDYDGDSYLNWYQQI |
| anti-CD19 | | PGQPPKLLIYDASNLVSGIPPRESGSGSGTDFTLNIHPVEKVDAA |
| | | TYHCQQSTEDPWTFGGGTKLEIK |
| HD37 VH | 30 | QVQLQQSGAELVRPGSSVKISCKASGYAFSSYWMNWVKQRPG |
| (anti- | | QGLEWIGQIWPGDGDTNYNGKFKGKATLTADESSSTAYMQLS |
| CD19) | | SLASEDSAVYFCARRETTTVGRYYYAMDYWGQGTTVTVSS |
| L2K07 | 31 | DIQLTQSPAIMSASPGEKVTMTCRASSSVSYMNWYQQKSGTSP |
| VL (anti- | | KRWIYDTSKVASGVPYRFSGSGSGTSYSLTISSMEAEDAATYY |
| CD3) | | CQQWSSNPLTFGAGTKLELK |
| L2K07 | 32 | DIKLQQSGAELARPGASVKMSCKTSGYTFTRYTMHWVKQRPG |
| VH (anti- | | QGLEWIGYINPSRGYTNYNQKFKDKATLTTDKSSSTAYMQLSS |
| CD3) | | LTSEDSAVYYCARYYDDHYCLDYWGQGTTLTVSS |
| FMC43 | 33 | DIQMTQTTSSLSASLGDRVTISCRASQDISKYLNWYQQKPDGT |
| VL (anti- | | VKLLIYHTSRLHSGVPSRFSGSGSGTDYSLTISNLEQEDIATYFC |
| CD19) | | QQGNTLPYTFGGGTKLEIT |
| FMC43 | 34 | EVKLQESGPGLVAPSQSLSVTCTVSGVSLPDYGVSWIRQPPRK |
| VH (anti- | | GLEWLGVIWGSETTYYNSALKSRLTIIKDNSKSQVFLKMNSLQ |
| CD19) | | TDDTAIYYCAKHYYYGGSYAMDYWGQGTSVTVSS |
| hSP34 VL | 35 | QTVVTQEPSLTVSPGGTVTLTCGSSTGAVTSGYYPNWVQQKP |
| (anti- | | GQAPRGLIGGTKFLAPGTPARFSGSLLGGKAALTLSGVQPEDE |
| CD3) | | AEYYCALWYSNRWVFGGGTKLTVL |
| hSP34 VH | 36 | EVQLVESGGGLVQPGGSLKLSCAASGFTFNKYAMNWVRQAP |
| (anti- | | GKGLEWVARIRSKYNNYATYYADSVKDRFTISRDDSKNTAYL |
| CD3) | | QMNNLKTEDTAVYYCVRHGNFGNSYISYWAYWGQGTLVTVS |
| | | S |
| BU12 VL | 37 | ENVLTQSPAIMSASPGEKVTMTCSASSSVSYMHWYQQKSSTSP |
| (anti- | | KLWIYDTSKLASGVPGRFSGSGSGNSHFLTISSMEAEDVATYY |
| CD19) | | CFQGSVYPFTFGSGTKLEIK |
| BU12 VH | 38 | QVTLKESGPGILQPSQTLSLTCSFSGFSLSTSGMGVGWIRQPSG |
| (anti- | | KGLEWLAHIWWDDDKRYNPALKSRLTISKDTSSNQVFLKIASV |
| CD19) | | DTADTAAYYCARMELWSYYFDYWGQGTTLTVSS |
| Signal | 39 | KLQDRLAKEGMHSSALLCCLVLLTGVRA |
| Peptide | | |
| Linker-1 | 40 | GGGGSGGGGSGGGGSGGGGS |
| Linker-2 | 41 | GGGGS |
| His-tag | 42 | HHHHHH |

In some embodiments, the signal peptide coding region comprises the sequence of SEQ ID NO: 11. In some embodiments, the synthetic RNA further comprises a linker coding region. In some embodiments, the linker coding region comprises the sequence of SEQ ID NO: 12 or SEQ ID NO: 13. In some embodiments, the therapeutic peptide coding region comprises nucleic acid sequences selected from the group consisting of: 1) SEQ ID NOs: 1 and 2; 2) SEQ ID NOs: 5 and 6; and 3) SEQ ID NOs: 9 and 10, wherein the nucleic acid sequences encode an anti-CD 19 antibody or anti-CD 19 binding fragments thereof.

In some embodiments, the therapeutic peptide coding region comprises nucleic acid sequences having 90% sequence identity to sequences selected from the group consisting of: 1) SEQ ID NOs: 1 and 2; 2) SEQ ID NOs: 5 and 6; or 3) SEQ ID NOs: 9 and 10, wherein the nucleic acid sequences encode an anti-CD 19 antibody or anti-CD 19 binding fragments thereof.

In some embodiments, the therapeutic peptide coding region comprises nucleic acid sequences consisting of: 1) SEQ ID NOs: 3 and 4; or 2) SEQ ID NOs: 7 and 8, wherein the nucleic acid sequences encode an anti-CD3 antibody or anti-CD3 binding fragments thereof.

In some embodiments, the therapeutic peptide coding region comprises nucleic acid sequences having 90% sequence identity to 1) SEQ ID NOs: 3 and 4; or 2) SEQ ID NOs: 7 and 8, wherein the nucleic acid sequences encode an anti-CD3 antibody or anti-CD3 binding fragments thereof.

In some embodiments, the synthetic nucleic acid sequence comprises a therapeutic peptide coding region. In some embodiments, the therapeutic peptide coding region comprises, in a 5′ to 3′ direction: a) an anti-CD19 light chain coding region; b) an anti-CD19 heavy chain coding region; c) an anti-CD3 heavy chain coding region; and d) an anti-CD3 light chain coding region.

In some embodiments, the anti-CD19 light chain coding region comprises the sequence of SEQ ID NOs: 1, 3, 5, or 9. In some embodiments, the anti-CD19 light chain coding region comprises nucleic acid sequences having 90% sequence identity to SEQ ID NOs: 1, 3, 5, or 9. In some embodiments, the anti-CD19 light chain coding region comprises nucleic acid sequences having at least 90%, 91%, 92%, 93%, 94%, 95%, 96%, 97%, 98%, 99%, or 100% sequence identity to SEQ ID NOs: 1, 3, 5, or 9.

In some embodiments, the anti-CD19 heavy chain coding region comprises the sequence of SEQ ID NOs: 2, 4, 6, or 10. In some embodiments, the anti-CD19 heavy chain coding region comprises nucleic acid sequences having 90% sequence identity to SEQ ID NOs: 2, 4, 6, or 10. In some embodiments, the anti-CD19 heavy chain coding region comprises nucleic acid sequences having at least 90%, 91%, 92%, 93%, 94%, 95%, 96%, 97%, 98%, 99%, or 100% sequence identity to SEQ ID NOs: 2, 4, 6, or 10.

In some embodiments, the anti-CD3 light chain coding region comprises the sequence of SEQ ID NO: 7. In some embodiments, the anti-CD3 light chain coding region comprises nucleic acid sequences having 90% sequence identity to SEQ ID NO: 7. In some embodiments, the anti-CD3 light chain coding region comprises nucleic acid sequences having at least 90%, 91%, 92%, 93%, 94%, 95%, 96%, 97%, 98%, 99%, or 100% sequence identity to SEQ ID NO: 7.

In some embodiments, the anti-CD3 heavy chain coding region comprises the sequence of SEQ ID NO: 8. In some embodiments, the anti-CD3 heavy chain coding region comprises nucleic acid sequences having 90% sequence identity to SEQ ID NO: 8. In some embodiments, the anti-CD3 heavy chain coding region comprises nucleic acid sequences having at least 90%, 91%, 92%, 93%, 94%, 95%, 96%, 97%, 98%, 99%, or 100% sequence identity to SEQ ID NO: 8.

In some embodiments, the anti-CD19 light chain coding region comprises SEQ ID NO: 1 and the anti-CD19 heavy chain coding region comprises SEQ ID NO: 2; wherein the anti-CD3 light chain coding region comprises SEQ ID NO: 3 and the anti-CD3 heavy chain coding region comprises SEQ ID NO: 4.

In some embodiments, the anti-CD19 light chain coding region comprises SEQ ID NO: 5 and the anti-CD19 heavy chain coding region comprises SEQ ID NO: 6; wherein the anti-CD3 light chain coding region comprises SEQ ID NO: 7 and the anti-CD3 heavy chain coding region comprises SEQ ID NO: 8.

In some embodiments, the anti-CD19 light chain coding region comprises SEQ ID NO: 9 and the anti-CD19 heavy chain coding region comprises SEQ ID NO: 10; wherein the anti-CD3 light chain coding region comprises SEQ ID NO: 7 and the anti-CD3 heavy chain coding region comprises SEQ ID NO: 8.

In some embodiments, the anti-CD19 heavy chain coding region consists of or comprises a nucleotide sequence that encodes a light chain of blinatumomab, inebilizumab, loncastuximab, or tafasitamab. In some embodiments, the anti-CD19 heavy chain coding region consists of or comprises a nucleotide sequence that encodes a heavy chain of blinatumomab, inebilizumab, loncastuximab, or tafasitamab. In some embodiments, the anti-CD3 light chain coding region consists of or comprises a nucleotide sequence that encodes a light chain of blinatumomab, inebilizumab, loncastuximab, or tafasitamab. In some embodiments, the anti-CD3 heavy chain coding region consists of or comprises a nucleotide sequence that encodes a heavy chain of blinatumomab, inebilizumab, loncastuximab, or tafasitamab.

In some embodiments, the synthetic RNA comprises a single strand synthetic RNA.

In some embodiments, the synthetic RNA further encodes a protein tag. In some embodiments, protein tag comprises a histidine tag, a flag tag or a hemagglutinin tag.

In some cases, the mRNA encodes a therapeutic peptide may comprise a signal peptide and the signal peptide can be cleaved during post-translational processing.

In some embodiments, the synthetic nucleic acid sequence comprises a therapeutic peptide coding region. In some embodiments, the therapeutic peptide coding region comprises, in a 5′ to 3′ direction: a) an anti-CD19 light chain coding region; b) an anti-CD19 heavy chain coding region; c) an anti-CD3 heavy chain coding region; and d) an anti-CD3 light chain coding region.

In some embodiments, the anti-CD19 light chain coding region comprises the sequence of SEQ ID NOs: 1, 3, 5, or 9. In some embodiments, the anti-CD19 light chain coding region comprises nucleic acid sequences having 90% sequence identity to SEQ ID NOs: 1, 3, 5, or 9. In some embodiments, the anti-CD19 light chain coding region comprises nucleic acid sequences having at least 90%, 91%, 92%, 93%, 94%, 95%, 96%, 97%, 98%, 99%, or 100% sequence identity to SEQ ID NOs: 1, 3, 5, or 9.

In some embodiments, the anti-CD19 heavy chain coding region comprises the sequence of SEQ ID NOs: 2, 4, 6, or 10. In some embodiments, the anti-CD19 heavy chain coding region comprises nucleic acid sequences having 90% sequence identity to SEQ ID NOs: 2, 4, 6, or 10. In some embodiments, the anti-CD19 heavy chain coding region comprises nucleic acid sequences having at least 90%, 91%, 92%, 93%, 94%, 95%, 96%, 97%, 98%, 99%, or 100% sequence identity to SEQ ID NOs: 2, 4, 6, or 10.

In some embodiments, the anti-CD3 light chain coding region comprises the sequence of SEQ ID NO: 7. In some embodiments, the anti-CD3 light chain coding region comprises nucleic acid sequences having 90% sequence identity to SEQ ID NO: 7. In some embodiments, the anti-CD3 light chain coding region comprises nucleic acid sequences having at least 90%, 91%, 92%, 93%, 94%, 95%, 96%, 97%, 98%, 99%, or 100% sequence identity to SEQ ID NO: 7.

In some embodiments, the anti-CD3 heavy chain coding region comprises the sequence of SEQ ID NO: 8. In some embodiments, the anti-CD3 heavy chain coding region comprises nucleic acid sequences having 90% sequence identity to SEQ ID NO: 8. In some embodiments, the anti-CD3 heavy chain coding region comprises nucleic acid sequences having at least 90%, 91%, 92%, 93%, 94%, 95%, 96%, 97%, 98%, 99%, or 100% sequence identity to SEQ ID NO: 8.

In some embodiments, the anti-CD19 light chain coding region comprises SEQ ID NO: 1 and the anti-CD19 heavy chain coding region comprises SEQ ID NO: 2; wherein the anti-CD3 light chain coding region comprises SEQ ID NO: 3 and the anti-CD3 heavy chain coding region comprises SEQ ID NO: 4.

In some embodiments, the anti-CD19 light chain coding region comprises SEQ ID NO: 5 and the anti-CD19 heavy chain coding region comprises SEQ ID NO: 6; wherein the anti-CD3 light chain coding region comprises SEQ ID NO: 7 and the anti-CD3 heavy chain coding region comprises SEQ ID NO: 8.

In some embodiments, the anti-CD19 light chain coding region comprises SEQ ID NO: 9 and the anti-CD19 heavy chain coding region comprises SEQ ID NO: 10; wherein the anti-CD3 light chain coding region comprises SEQ ID NO: 7 and the anti-CD3 heavy chain coding region comprises SEQ ID NO: 8.

In some embodiments, the anti-CD19 heavy chain coding region consists of or comprises a nucleotide sequence that encodes a light chain of blinatumomab, inebilizumab, loncastuximab, or tafasitamab. In some embodiments, the anti-CD19 heavy chain coding region consists of or comprises a nucleotide sequence that encodes a heavy chain of blinatumomab, inebilizumab, loncastuximab, or tafasitamab. In some embodiments, the anti-CD3 light chain coding region consists of or comprises a nucleotide sequence that encodes a light chain of blinatumomab, inebilizumab, loncastuximab, or tafasitamab. In some embodiments, the anti-CD3 heavy chain coding region consists of or comprises a nucleotide sequence that encodes a heavy chain of blinatumomab, inebilizumab, loncastuximab, or tafasitamab.

In some cases, the mRNA comprises a hepatocyte-specific promoter and the LNPs comprising the mRNA encoding a soluble therapeutic peptide (e.g., BiTE). In some cases, the mRNA comprises a strong promoter that enhances the expression of the therapeutic peptide for immunotherapy. The strong promoter can comprise a Human cytomegalovirus (hCMV), chicken beta-actin/CMV enhancer (CAG), elongation factor-1alpha (EF1α), or phosphoglycerokinase (PGK) promoter.

In some cases, the mRNA encapsulated by the LNPs of the present disclosure comprises a naturally occurring or an artificial promoter. In some cases, the mRNA comprises a promoter that is specific for expression in immune cells as compared to non-immune cells. In some cases, the mRNA comprises a T-cell specific promoter and the LNPs comprising the mRNA encoding a therapeutic peptide can be used for CAR-T therapy. In some cases, the T-cell specific promoter comprises promoters that drive endogenous expression of T-cell specific proteins comprising CD3 (e.g., CD3 gamma, CD3 delta, CD3 epsilon, CD3 zeta), CD4, CD8, CD28, TCRB or TRAC. In some cases, the promoter can result in stronger expression of therapeutic peptides in immune cells comprising lymphocytes, T cells, CD4+ T cells, CD8+ T cells, alpha-beta T cells, gamma-delta T cells, T regulatory cells (Tregs), cytotoxic T lymphocytes, Th1 cells, Th2 cells, Th17 cells, Th9 cells, naïve T cells, memory T cells, effector T cells, effector-memory T cells (TEM), central memory T cells (TCM), resident memory T cells (TRM), follicular helper T cells (TFH), Natural killer T cells (NKTs), tumor-infiltrating lymphocytes (TILs), Natural killer cells (NKs), Innate Lymphoid Cells (ILCs), ILC1 cells, ILC2 cells, ILC3 cells, lymphoid tissue inducer (LTi) cells, B cells, B1 cells, B1a cells, B1b cells, B2 cells, plasma cells, B regulatory cells, memory B cells, marginal zone B cells, follicular B cells, germinal center B cells, antigen presenting cells (APCs), monocytes, macrophages, M1 macrophages, M2 macrophages, tissue-associated macrophages, dendritic cells, plasmacytoid dendritic cells, neutrophils, mast cells, basophils, eosinophils, common myeloid progenitors, common lymphoid progenitors, or any combination thereof.

In some cases, an mRNA encoding a therapeutic peptide disclosed herein comprises natural, synthetic, and/or artificial nucleotide analogues or bases. In some cases, the synthetic or artificial nucleotide analogues or bases comprise modifications at one or more of deoxyribose moieties, ribose moiety, phosphate moiety, nucleoside moiety, or a combination thereof.

In some cases, a nucleotide analogue or artificial nucleotide base comprises a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Illustrative alkyl moiety includes, but are not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some cases, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety.

In some cases, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties.

In some cases, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer.

In some cases, additional modifications at the 2′ hydroxyl group include 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA).

In some cases, a nucleotide analogue comprises a modified base, for example, N1-methylpseudouridine, 5-propynyluridine, 5-propynylcytidine, 6-methyladenine, 6-methylguanine, N, N-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes universal bases. By way of example, universal bases include but are not limited to 3-nitropyrrole, 5-nitroindole, or nebularine.

In some cases, one or more modifications optionally occur at the internucleotide linkage. In some instances, a modified internucleotide linkages can include, but is not limited to, phosphorothioates; phosphorodithioates; methylphosphonates; 5′-alkylenephosphonates; 5′-methylphosphonate; 3′-alkylene phosphonates; borontrifluoridates; borano phosphate esters and selenophosphates of 3′-5′linkage or 2′-5′linkage; phosphotriesters; thionoalkylphosphotriesters; hydrogen phosphonate linkages; alkyl phosphonates; alkylphosphonothioates; arylphosphonothioates; phosphoroselenoates; phosphorodiselenoates; phosphinates; phosphoramidates; 3′-alkylphosphoramidates; aminoalkylphosphoramidates; thionophosphoramidates; phosphoropiperazidates; phosphoroanilothioates; phosphoroanilidates; ketones; sulfones; sulfonamides; carbonates; carbamates; methylenehydrazos; methylenedimethylhydrazos; formacetals; thioformacetals; oximes; methyleneiminos; methylenemethyliminos; thioamidates; linkages with riboacetyl groups; aminoethyl glycine; silyl or siloxane linkages; alkyl or cycloalkyl linkages with or without heteroatoms of, for example, 1 to 10 carbons that are saturated or unsaturated and/or substituted and/or contain heteroatoms; linkages with morpholino structures, amides, or polyamides wherein the bases are attached to the aza nitrogens of the backbone directly or indirectly; and combinations thereof.

In some cases, one or more modifications comprise a modified phosphate backbone in which the modification generates a neutral or uncharged backbone. In some instances, the phosphate backbone is modified by alkylation to generate an uncharged or neutral phosphate backbone. As used herein, alkylation includes methylation, ethylation, and propylation. In some cases, an alkyl group, as used herein in the context of alkylation, refers to a linear or branched saturated hydrocarbon group containing from 1 to 6 carbon atoms. In some instances, exemplary alkyl groups include, but are not limited to, methyl, ethyl, n-propyl, isopropyl, n-butyl, isobutyl, sec-butyl, tert-butyl, n-pentyl, isopentyl, neopentyl, hexyl, isohexyl, 1,1-dimethylbutyl, 2,2-dimethylbutyl, 3.3-dimethylbutyl, and 2-ethylbutyl groups. In some cases, a modified phosphate is a phosphate group as described in U.S. Pat. No. 9,481,905.

In some embodiments, additional modified phosphate backbones comprise methylphosphonate, ethylphosphonate, methylthiophosphonate, or methoxyphosphonate. In some cases, the modified phosphate is methylphosphonate. In some cases, the modified phosphate is ethylphosphonate. In some cases, the modified phosphate is methylthiophosphonate. In some cases, the modified phosphate is methoxyphosphonate.

In some cases, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

### Lipid Compositions

In one aspect, provided herein is a pharmaceutical composition comprising a synthetic nucleic acid sequence assembled with a lipid composition, wherein the lipid composition comprises an ionizable lipid.

In some embodiments, the composition comprises a synthetic nucleic acid sequence assembled with a lipid composition, and the lipid composition comprises an ionizable lipid, wherein the ionizable lipid comprises an amine head group and at least one hydrophobic tail R^{Lipid} having a structure of

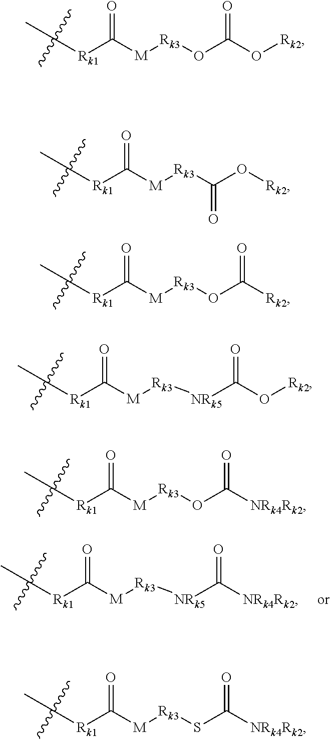

or a pharmaceutically acceptable salt thereof; wherein
Rₖ₁ is independently a C1-C12 bivalent aliphatic or heteroaliphatic radical;
Rₖ₃ is independently a C1-C12 alkyl, C2-C12 alkenyl, C2-C12 alkynyl, C3-C12 cycloalkyl, C3-C12 heterocycloalkyl, aryl, or heteroaryl;
Rₖ₂ is independently a C1-C20 alkyl, C2-C20 alkenyl, C2-C20 alkynyl, C3-C20 cycloalkyl, C1-C20 heteroalkyl, C3-C20 heterocycloalkyl, aryl, or heteroaryl;
Rₖ₄ and Rₖ₅ are each independently H or C1-C12 bivalent aliphatic radical;
M is O or NRₖ₆, wherein Rₖ₆ is H or C1-C12 aliphatic radical; and the synthetic nucleic acid sequence may encode one or more therapeutic peptides, wherein the therapeutic peptide binds to a B cell antigen.

In some embodiments, the ionizable lipid comprises an amine head group and at least one hydrophobic tail R^{Lipid} having a structure of Formula (I):

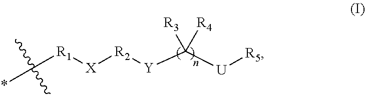

or a pharmaceutically acceptable salt thereof;
wherein:
* indicates a point of attachment to a nitrogen in the amine head group;
R₁ and R₂ are each independently a C1-C12 bivalent aliphatic or heteroaliphatic radical;
X is

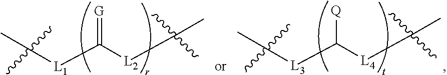

in which each of L₁, L₂, L₃, and L₄ is, independently, a bond, O, S, or NRC; G is 0, S, or NR^{d}; Q is OR^{e}, SR^{f}, or NR^{g}R^{h}; and
each of r and t is independently 1-6;
each of R^{e}, R^{d}, R^{e}, R^{f}, R^{g}, and R^{h} is independently H, C1-C10 alkyl, C1-C10 heteroalkyl, aryl, or heteroaryl;
Y and U are each independently a bond, O, S, NR¹⁰, or Se;
n is 0 or 1;
R₃ and R₄, are each independently H, C1-C10 alkyl, C1-C10 heteroalkyl, aryl, or heteroaryl; or R₃ and R₄ together with the atom to which they are attached, form C═O;
R₅ is a C1-C20 alkyl, C2-C20 alkenyl, C2-C20 alkynyl, C3-C20 cycloalkyl, C1-C20 heteroalkyl, C3-C20 heterocycloalkyl, aryl, or heteroaryl; and
the synthetic nucleic acid sequence may encode one or more therapeutic peptides, wherein the therapeutic peptide binds to a B cell antigen.

In some embodiments, n is 0. In some embodiments, n is 1.

In some embodiments, n is 1 and R₃ and R₄ together with the atom to which they are attached, form C═O. In some embodiments, Y is CH₂ and U is O. In some embodiments, Y and U are both O. In some embodiments, U is CH₂ and Y is O. In some embodiments, Y and U are NR¹⁰. In some embodiments, Y is O and U is NR¹⁰.

In some embodiments, Y is S and U is NR¹⁰.

In some embodiments, n is 0, and Y and U are both S.

In some embodiments, n is 0, and one of Y and U is O.

In some embodiments, n is 0, and one of Y and U is Se.

In some embodiments, R₁ is a C1-C12 alkyl, linear or branched. In some embodiments, R₁ is a C1-C10 alkyl, linear or branched. In some embodiments, R₁ is a C1-C8 alkyl, linear or branched. In some embodiments, R₁ is a C1-C6 alkyl, linear or branched. In some embodiments, R₁ is a C1-C4 alkyl, linear or branched. In some embodiments, R₁ is a C2 alkyl, e.g.,

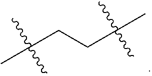 [SMILES: *CC*]

In some embodiments, R₁ is a C3 alkyl, e.g.,

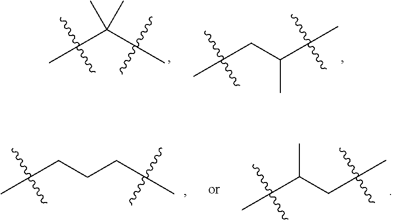

In some embodiments, R₁ is a C4 alkyl. In some embodiments, R₁ is a C1-C12 heteroaliphatic radical.

In some embodiments, R₂ is a C1-C12 alkyl, linear or branched. In some embodiments, R₂ is a C1-C10 alkyl, linear or branched. In some embodiments, R₂ is a C1-C8 alkyl, linear or branched. In some embodiments, R₂ is a C1-C6 alkyl, linear or branched. In some embodiments, R₂ is a C1-C4 alkyl, linear or branched. In some embodiments, R₂ is a C2 alkyl, e.g.,

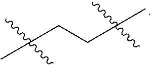 [SMILES: *CC*]

In some embodiments, R₂ is a C3 alkyl, e.g.,

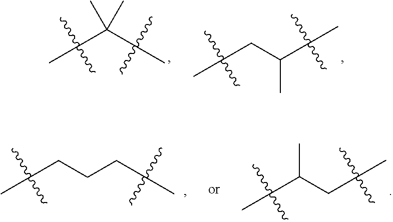

In some embodiments, R₂ is a C4 alkyl. In some embodiments, R₂ is a C1-C12 heteroaliphatic radical.

In some embodiments, the amine head group is represented by

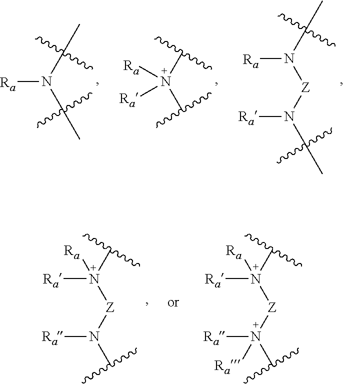

wherein Ra, Ra′, Ra″, and Ra′″ are each independently, H, C1-20 alkyl, C2-C20 alkenyl, C2-C20 alkynyl, C3-C20 cycloalkyl or heterocycloalkyl, C1-C20 heteroalkyl, C3-C20 aryl or heteroaryl, or a R^{Lipid},

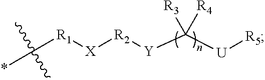

and Z is a C1-C20 bivalent aliphatic radical, a C1-C20 bivalent heteroaliphatic radical, a bivalent aryl radical, or a bivalent heteroaryl radical.

In some embodiments, the ionizable lipid is represented by Formula (II):

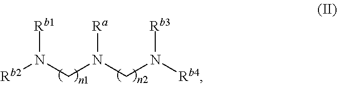

or a pharmaceutically acceptable salt thereof, wherein:
   i) R^{b} is a substituted or unsubstituted alkyl, hydroxyalkyl, alkoxyalkyl, or aryl;
   ii) n1 and n2 are each independently 1, 2, 3, 4, 5, or 6; and
   iii) R^{b1}, R^{b2}, R^{b3} and R^{b4} are each independently H, or R^{Lipid}

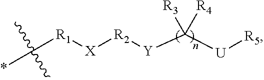

wherein at least one of R^{b1}, R^{b2}, R^{b3} and R^{b4} is not H.

In some embodiments, R^{b} is a C1-C6 alkyl, linear or branched. In some embodiments, R^{b} is a substituted C1-C6 alkyl, linear or branched. In some embodiments, a substituent comprises hydroxyl, carbonyl, thiocarbonyl, alkoxyl, phosphoryl, phosphate, phosphonate, phosphinate, amino, amide, cyclic amine, amidine, imine, cyano, nitro, azido, sulfhydryl, alkylthio, sulfate, sulfonate, sulfamoyl, sulfonamido, sulfonyl, heterocyclyl, aralkyl, or aromatic or heteroaromatic moiety. In some embodiments, the substituent comprises hydroxyl, NH-Boc,

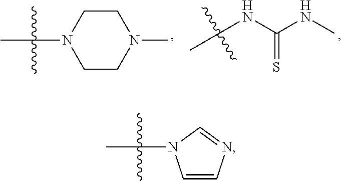

or any suitable substituent thereof.

In some embodiments, n1 is 1, 2, 3, 4, 5, or 6. In some embodiments, n1 is 2 or 3.

In some embodiments, n2 is 1, 2, 3, 4, 5, or 6. In some embodiments, n2 is 2 or 3.

In some embodiments, n1 and n2 are identical. In some embodiments, n1 and n2 are different. In some embodiments, both n1 and n2 are 2. In some embodiments, both n1 and n2 are 3. In some embodiments, both n1 and n2 are 4.

In some embodiments, R^{b1} is not H. In some embodiments, R^{b2} is not H. In some embodiments, R^{b3} is not H. In some embodiments, R^{b4} is not H.

In some embodiments, at least two of R^{b1}, R^{b2}, R^{b3} and R^{b4} are not H. In some embodiments, at least three of R^{b1}, R^{b2}, R^{b3} and R^{b4} are not H. In some embodiments, none of R^{b1}, R^{b2}, R^{b3} and R^{b4} is H.

In some embodiments, the amine head group is selected from the group consisting of

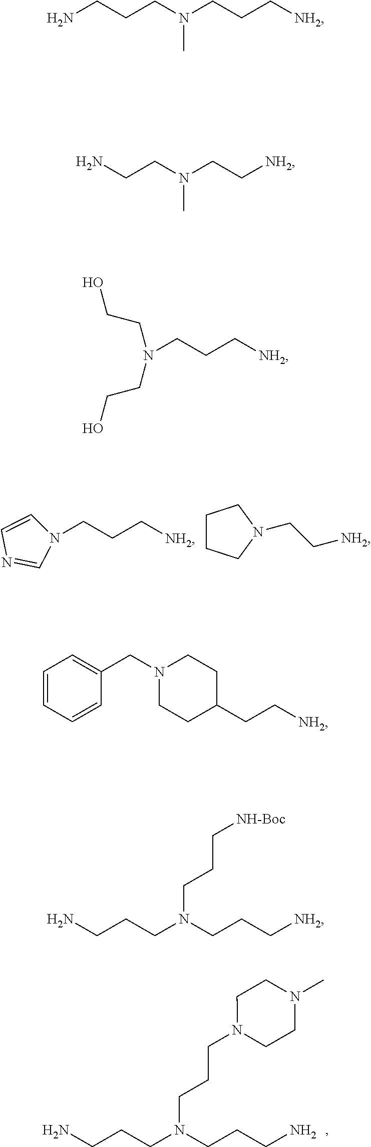

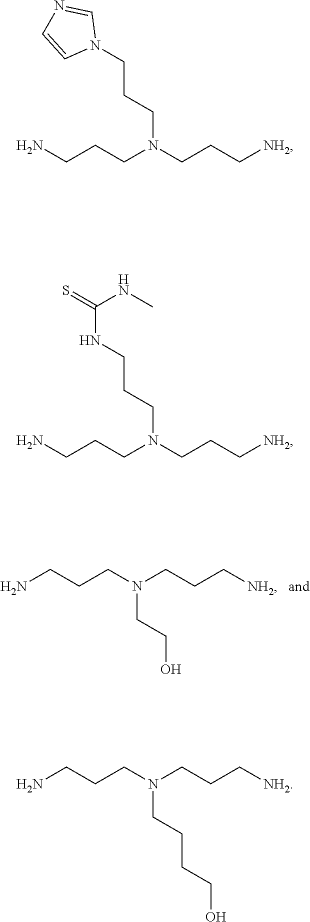 [SMILES: NCCCN(CCCN)CCCn1ccnc1 ; CNC(=S)NCCCN(CCCN)CCCN ; NCCCN(CCO)CCCN ; "and" ; NCCCN(CCCN)CCCCO]

In some embodiments, the at least one hydrophobic tail has a structure of

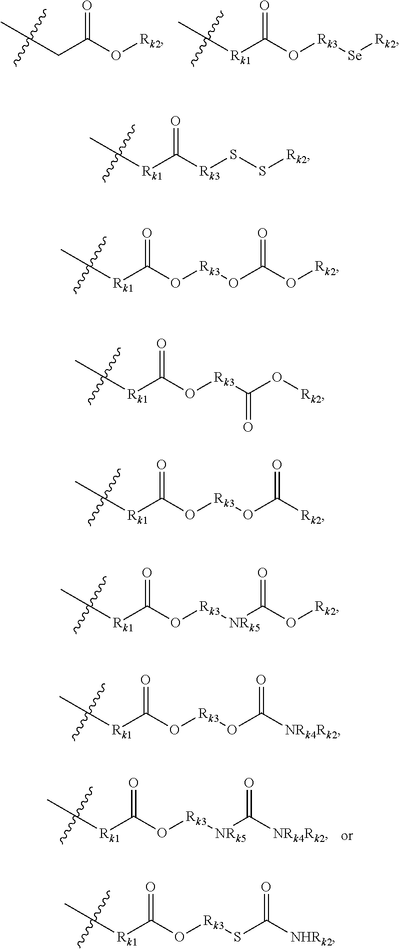

wherein Rₖ₁ and Rₖ₃ are each independently a C1-C10 alkyl;
Rₖ₂ is a C1-C20 alkyl, C2-C20 alkenyl, C2-C20 alkynyl, C3-C20 cycloalkyl, C1-C20 heteroalkyl, C3-C20 heterocycloalkyl, aryl, or heteroaryl;
Rₖ₄ and Rₖ₅ are each independently H, or C1-C10 alkyl.

In some embodiments, Rₖ₁ is a C1-C10 alkyl, linear or branched. In some embodiments, Rₖ₁ is a C1-C4 alkyl, linear or branched. In some embodiments, Rₖ₁ is a C2 alkyl, e.g.,

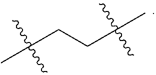 [SMILES: *CC*]

In some embodiments, Rₖ₁ is a C3 alkyl, e.g.,

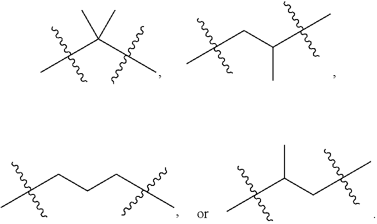

In some embodiments, Rₖ₁ is a C4 alkyl.

In some embodiments, Rₖ₃ is a C1-C10 alkyl, linear or branched. In some embodiments, Rₖ₃ is a C1-C4 alkyl, linear or branched. In some embodiments, Rₖ₃ is a C2 alkyl, e.g.,

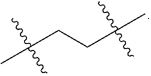 [SMILES: *CC*]

In some embodiments, Rₖ₃ is a C3 alkyl, e.g.,

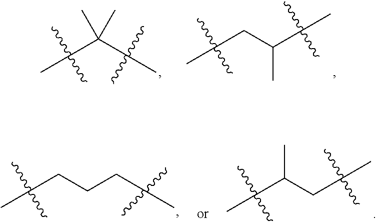

In some embodiments, Rₖ₃ is a C4 alkyl.

In some embodiments, Rₖ₄ is H. In some embodiments, Rₖ₄ is C1-C10 alkyl. In some embodiments, Rₖ₄ is C1-C4 alkyl. In some embodiments, Rₖ₄ is C4-C10 alkyl.

In some embodiments, Rₖs is H. In some embodiments, Rₖ₅ is C1-C10 alkyl. In some embodiments, Rₖ₅ is C1-C4 alkyl. In some embodiments, Rₖs is C4-C10 alkyl.

In some embodiments, Rₖ₂ is a C1-C20 alkyl. In some embodiments, Rₖ₂ is a C2-C20 alkenyl. In some embodiments, Rₖ₂ is a C2-C20 alkynyl. In some embodiments, Rₖ₂ is a C3-C20 cycloalkyl. In some embodiments, Rₖ₂ is a C1-C20 heteroalkyl. In some embodiments, Rₖ2 is a C3-C20 heterocycloalkyl, aryl, or heteroaryl.

In some embodiments, the at least one hydrophobic tail comprises

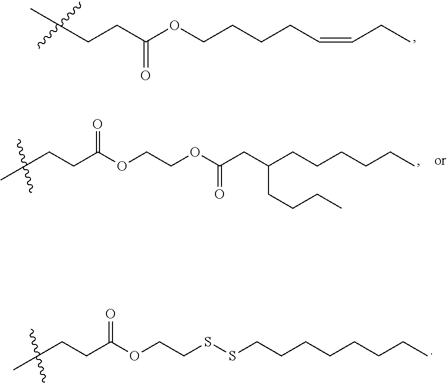 [SMILES: *CCC(=O)OCCCC/C=C\CC ; *CCC(=O)OCCOC(=O)CC(CCCC)CCCCCC ; *CCC(=O)OCCSSCCCCCCCC]

In some embodiments, the at least one hydrophobic tail is selected from the TABLE 2.

TABLE 2
Exemplary Hydrophobic Tail

| Tail # | Tail Structure |
|---|---|
| T0060 | 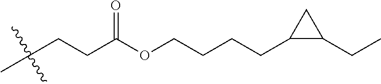 [SMILES: *CCC(=O)OCCCCC1CC1CC] |
| T0061 | 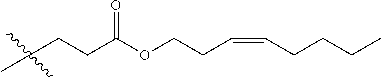 [SMILES: *CCC(=O)OCC/C=C\CCCC] |
| T0062 | 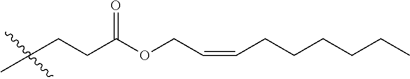 [SMILES: *CCC(=O)OC/C=C\CCCCCC] |
| T0063 | 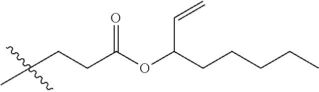 [SMILES: *CCC(=O)OC(C=C)CCCCC] |
| T0064 | 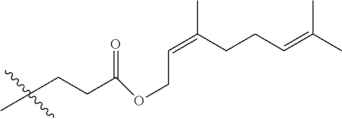 [SMILES: *CCC(=O)OC/C=C(/C)CCC=C(C)C] |
| T0065 | 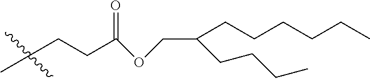 [SMILES: *CCC(=O)OCC(CCCC)CCCCCC] |
| T0066 | 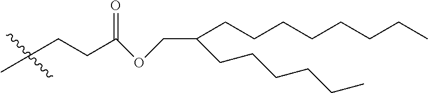 [SMILES: *CCC(=O)OCC(CCCCCC)CCCCCCCC] |
| T0068 | 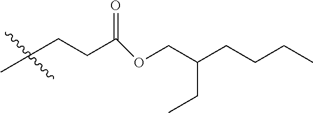 [SMILES: *CCC(=O)OCC(CC)CCCC] |
| T0069 | 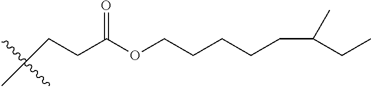 [SMILES: *CCC(=O)OCCCCCC(C)CC] |
| T0070 | 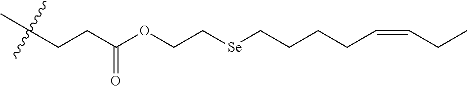 [SMILES: *CCC(=O)OCC[Se]CCCC/C=C\CC] |
| T0007 | 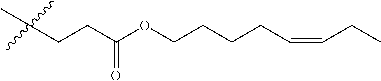 [SMILES: *CCC(=O)OCCCC/C=C\CC] |
| T0114 | 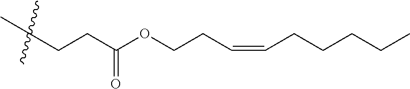 [SMILES: *CCC(=O)OCC/C=C\CCCCC] |
| T0115 | 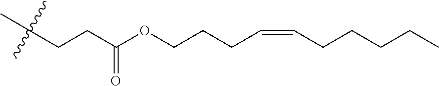 [SMILES: *CCC(=O)OCCC/C=C\CCCCC] |
| T0088 | 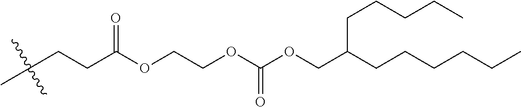 [SMILES: *CCC(=O)OCCOC(=O)OCC(CCCCC)CCCCCC] |
| T0075 | 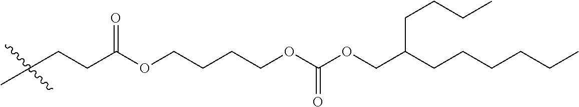 [SMILES: *CCC(=O)OCCCCOC(=O)OCC(CCCC)CCCCCC] |
| T0076 | 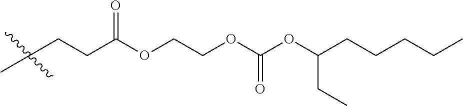 [SMILES: *CCC(=O)OCCOC(=O)OC(CC)CCCCC] |
| T0077 | 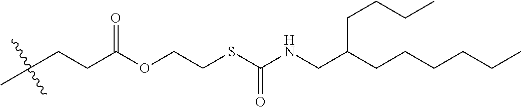 [SMILES: *CCC(=O)OCCSC(=O)NCC(CCCC)CCCCCC] |
| T0122 | 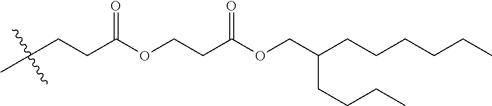 [SMILES: *CCC(=O)OCCC(=O)OCC(CCCC)CCCCCC] |
| T0128 | 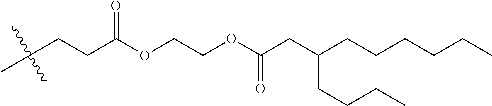 [SMILES: *CCC(=O)OCCOC(=O)CC(CCCC)CCCCCC] |
| T0082 | 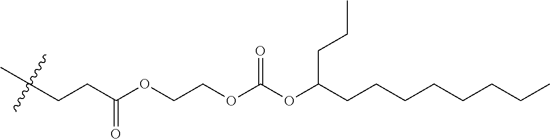 [SMILES: *CCC(=O)OCCOC(=O)OC(CCC)CCCCCCCC] |
| T0083 | 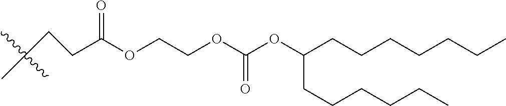 [SMILES: *CCC(=O)OCCOC(=O)OC(CCCCCC)CCCCCCC] |
| T0084 | 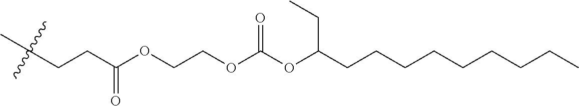 [SMILES: *CCC(=O)OCCOC(=O)OC(CC)CCCCCCCCC] |
| T0071 | 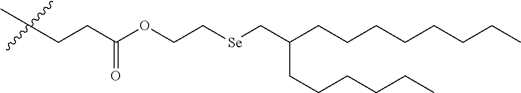 [SMILES: *CCC(=O)OCC[Se]CC(CCCCCC)CCCCCCCC] |
| T0072 | 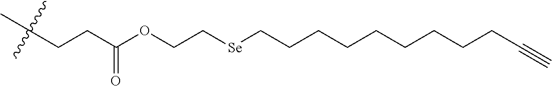 [SMILES: *CCC(=O)OCC[Se]CCCCCCCCCC#C] |
| T0079 | 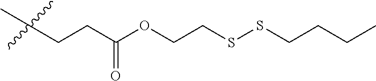 [SMILES: *CCC(=O)OCCSSCCCC] |
| T0089 | 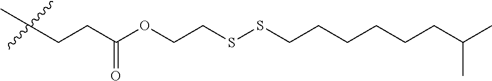 [SMILES: *CCC(=O)OCCSSCCCCCCC(C)C] |
| T0092 | 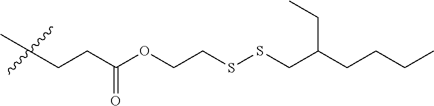 [SMILES: *CCC(=O)OCCSSCC(CC)CCCC] |
| T0093 | 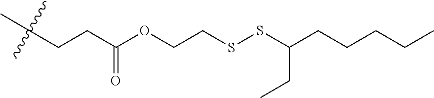 [SMILES: *CCC(=O)OCCSSC(CC)CCCCC] |
| T0106 | 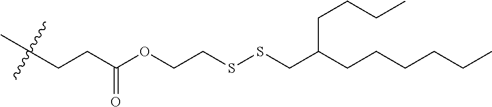 [SMILES: *CCC(=O)OCCSSCC(CCCC)CCCCCC] |
| T0108 | 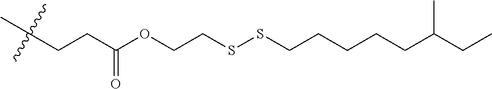 [SMILES: *CCC(=O)OCCSSCCCCCC(C)CC] |
| T0110 | 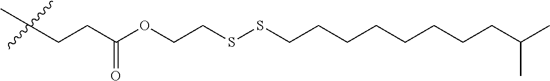 [SMILES: *CCC(=O)OCCSSCCCCCCCCC(C)C] |
| T0113 | 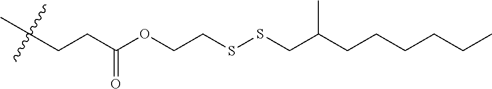 [SMILES: *CCC(=O)OCCSSCC(C)CCCCCC] |
| T0001 | 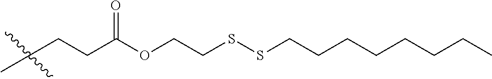 [SMILES: *CCC(=O)OCCSSCCCCCCCC] |
| T0097 | 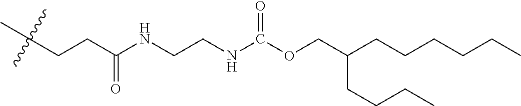 [SMILES: *CCC(=O)NCCNC(=O)OCC(CCCC)CCCCCC] |
| T0094 | 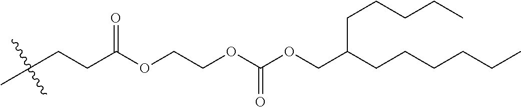 [SMILES: *CCC(=O)OCCOC(=O)OCC(CCCCC)CCCCCC] |
| T0096 | 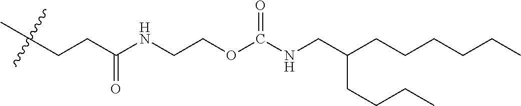 [SMILES: *CCC(=O)NCCOC(=O)NCC(CCCC)CCCCCC] |
| T0100 | 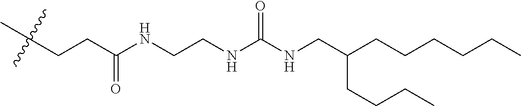 [SMILES: *CCC(=O)NCCNC(=O)NCC(CCCC)CCCCCC] |

In some embodiments, the ionizable lipid comprises:

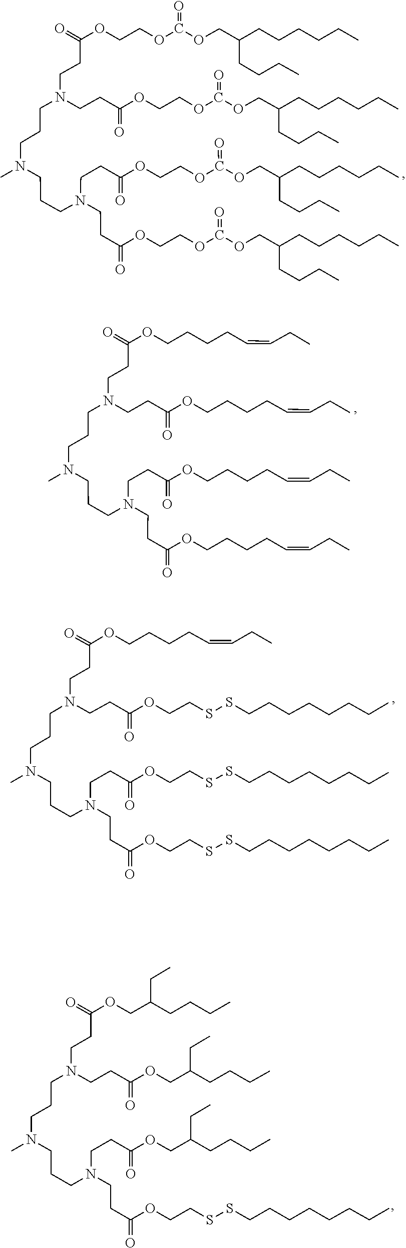

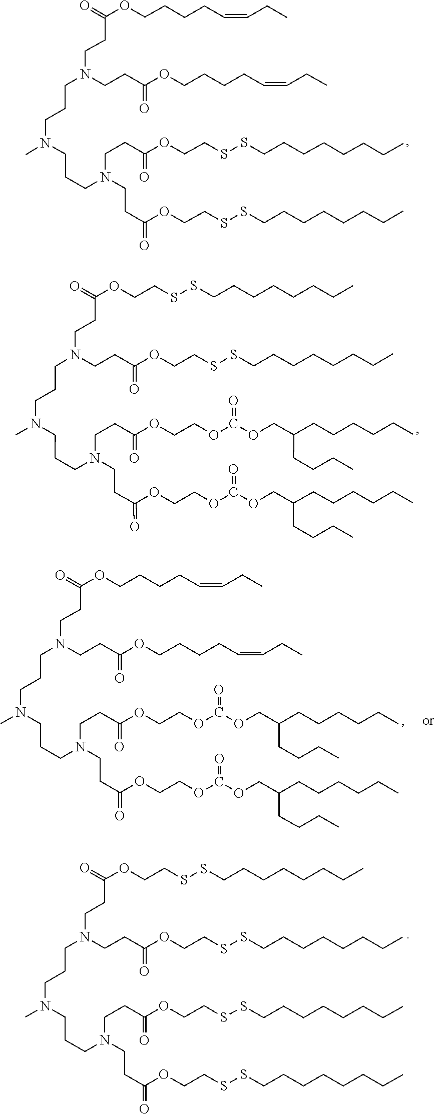

In some embodiments, the ionizable lipid comprises at least two hydrophobic tails. In some embodiments, the at least two hydrophobic tails are independently of structure

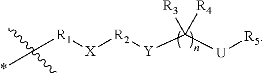

In some embodiments, the at least two hydrophobic tails are identical. In some embodiments, the at least two hydrophobic tails are not identical. In some embodiments, one of the at least two hydrophobic tails is different from the rest.

In some embodiments, the lipid composition comprises at least three hydrophobic tails. In some embodiments, the at least three hydrophobic tails are independently of structure

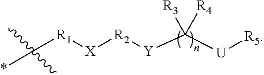

In some embodiments, the at least three hydrophobic tails are identical. In some embodiments, the at least three hydrophobic tails are not identical. In some embodiments, one of the at least three hydrophobic tails is different from the rest.

In some embodiments, the lipid composition comprises two hydrophobic tails. In some embodiments, the two hydrophobic tails are independently of structure

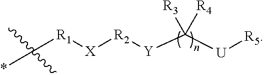

In some embodiments, the two hydrophobic tails are identical. In some embodiments, the two hydrophobic tails are not identical.

In some embodiments, the lipid composition comprises three hydrophobic tails. In some embodiments, the three hydrophobic tails are independently of structure

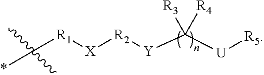

In some embodiments, the three hydrophobic tails are identical. In some embodiments, two of the three hydrophobic tails are identical and the third hydrophobic tail is different. In some embodiments, all three hydrophobic tails are different.

In some embodiments, the lipid composition comprises four hydrophobic tails. In some embodiments, the four hydrophobic tails are independently of structure

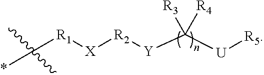

In some embodiments, the four hydrophobic tails are identical. In some embodiments, three of the four hydrophobic tails are identical and the fourth hydrophobic tail is different. In some embodiments, two of the four hydrophobic tails are identical, the other two hydrophobic tails are identical, and the two are different from other two. In some embodiments, two of the four hydrophobic tails are identical while the other two are different from each other and are different from the two. In some embodiments, all four hydrophobic tails are different.

In some embodiments, the ionizable lipid is selected from TABLE3.

TABLE 3
Exemplary Ionizable Lipid

| Lipid# | Lipid Structure |
|---|---|
| L0522 | 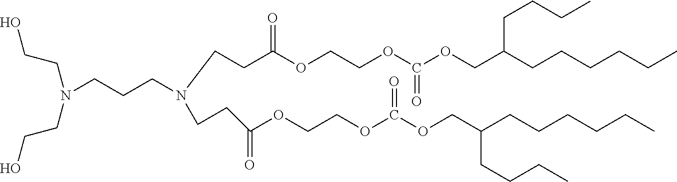 [SMILES: CCCCCCC(CCCC)COC(=O)OCCOC(=O)CCN(CCCN(CCO)CCO)CCC(=O)OCCOC(=O)OCC(CCCC)CCCCCC] |
| L0524 | 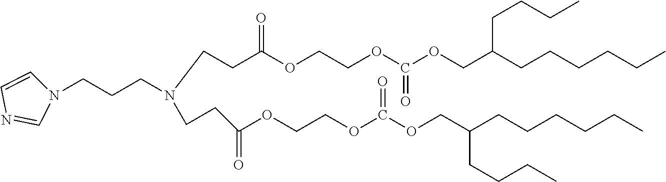 [SMILES: CCCCCCC(CCCC)COC(=O)OCCOC(=O)CCN(CCCn1ccnc1)CCC(=O)OCCOC(=O)OCC(CCCC)CCCCCC] |
| L0526 | 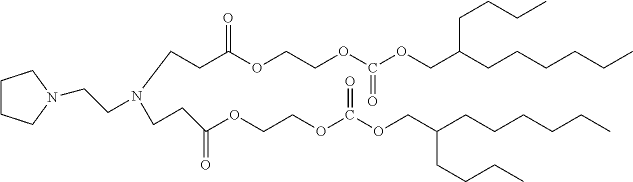 [SMILES: CCCCCCC(CCCC)COC(=O)OCCOC(=O)CCN(CCC(=O)OCCOC(=O)OCC(CCCC)CCCCCC)CCN1CCCC1] |
| L0539 | 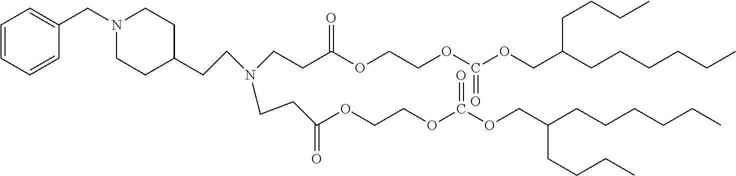 [SMILES: CCCCCCC(CCCC)COC(=O)OCCOC(=O)CCN(CCC(=O)OCCOC(=O)OCC(CCCC)CCCCCC)CCC1CCN(Cc2ccccc2)CC1] |
| L0668 | 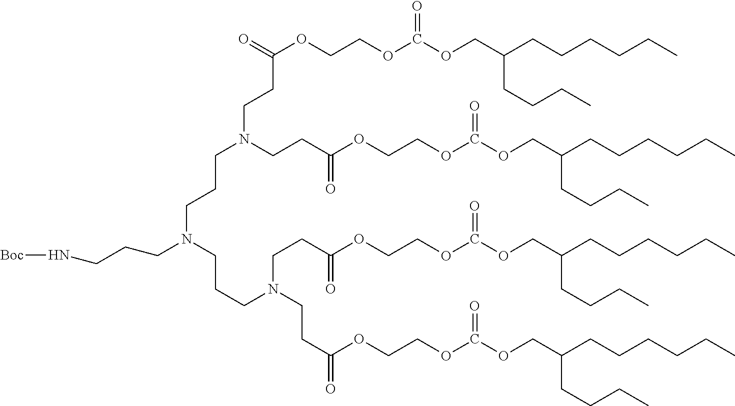 [SMILES: CCCCCCC(CCCC)COC(=O)OCCOC(=O)CCN(CCCN(CCCNC(=O)OC(C)(C)C)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0674 | 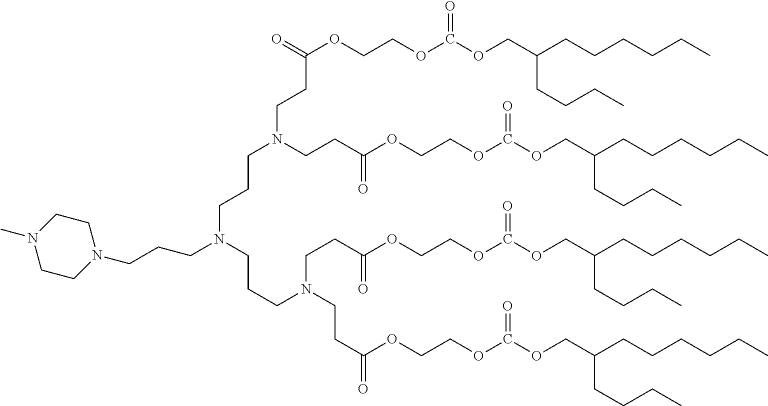 [SMILES: CCCCCCC(CCCC)COC(=O)OCCOC(=O)CCN(CCCN(CCCN(CCC(=O)OCCOC(=O)OCC(CCCC)CCCCCC)CCC(=O)OCCOC(=O)OCC(CCCC)CCCCCC)CCCN1CCN(C)CC1)CCC(=O)OCCOC(=O)OCC(CCCC)CCCCCC] |
| L0773 | 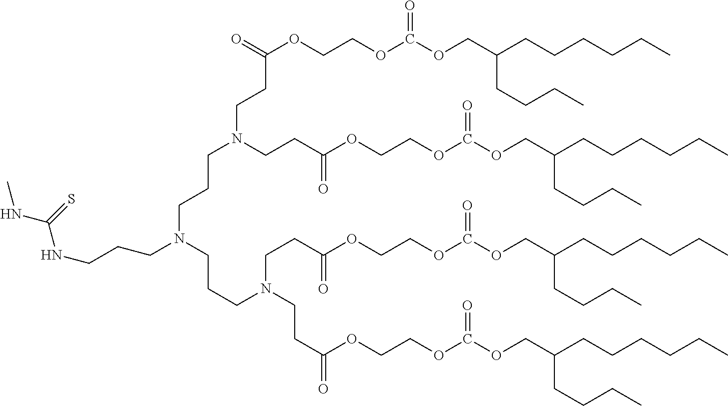 [SMILES: CCCCCCC(CCCC)COC(=O)OCCOC(=O)CCN(CCCN(CCCNC(=S)NC)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0665 | 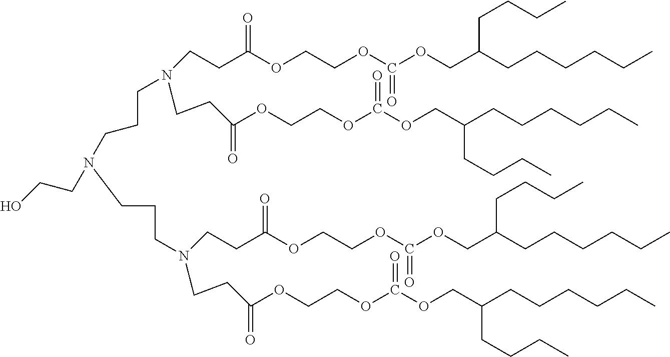 [SMILES: CCCCCCC(CCCC)COC(=O)OCCOC(=O)CCN(CCCN(CCO)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0666 | 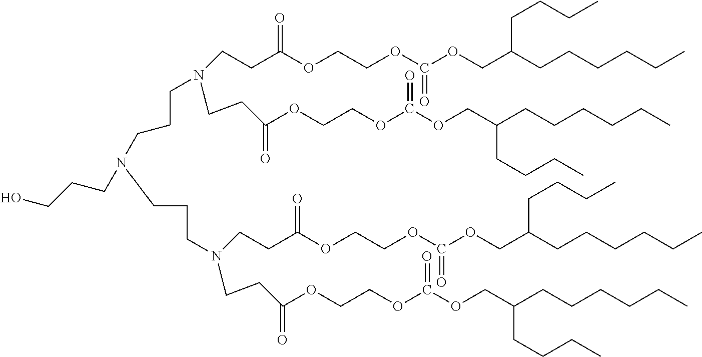 [SMILES: CCCCCCC(CCCC)COC(=O)OCCOC(=O)CCN(CCCN(CCCO)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0667 | 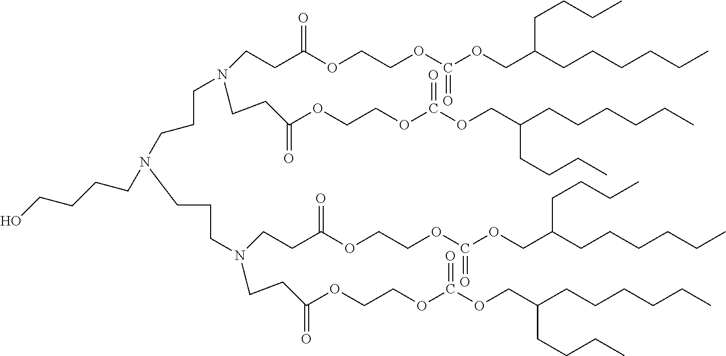 [SMILES: CCCCCCC(CCCC)COC(=O)OCCOC(=O)CCN(CCCN(CCCCO)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0586 | 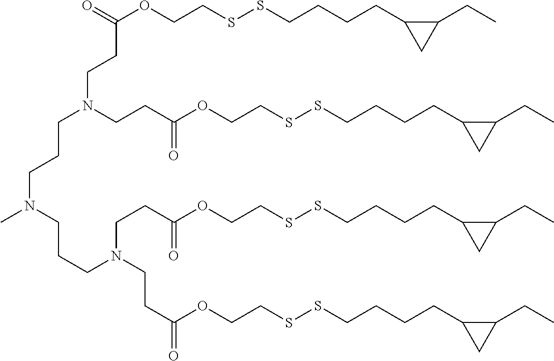 [SMILES: CCC1CC1CCCCSSCCOC(=O)CCN(CCCN(C)CCCN(CCC(=O)OCCSSCCCCC1CC1CC)CCC(=O)OCCSSCCCCC1CC1CC)CCC(=O)OCCSSCCCCC1CC1CC] |
| L0587 | 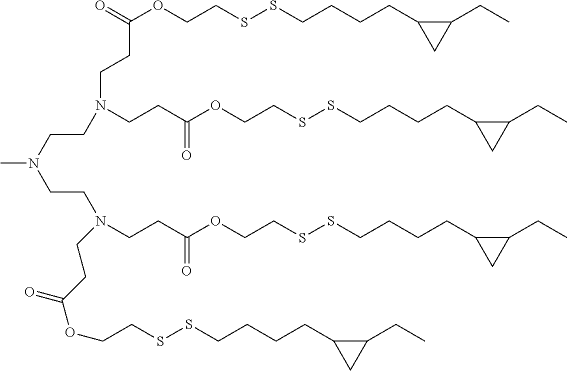 [SMILES: CCC1CC1CCCCSSCCOC(=O)CCN(CCC(=O)OCCSSCCCCC1CC1CC)CCN(C)CCN(CCC(=O)OCCSSCCCCC1CC1CC)CCC(=O)OCCSSCCCCC1CC1CC] |
| L0588 | 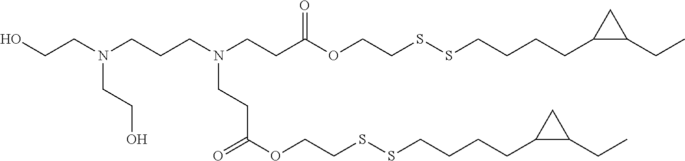 [SMILES: CCC1CC1CCCCSSCCOC(=O)CCN(CCCN(CCO)CCO)CCC(=O)OCCSSCCCCC1CC1CC] |
| L0589 | 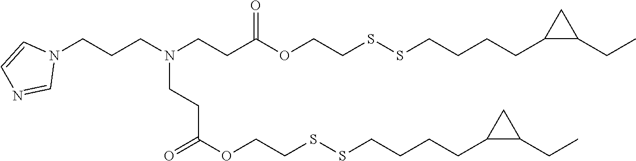 [SMILES: CCC1CC1CCCCSSCCOC(=O)CCN(CCCn1ccnc1)CCC(=O)OCCSSCCCCC1CC1CC] |
| L0591 | 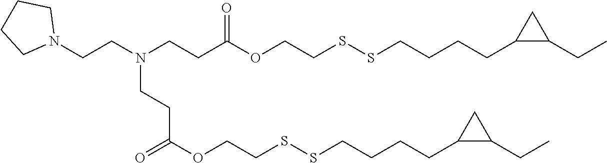 [SMILES: CCC1CC1CCCCSSCCOC(=O)CCN(CCC(=O)OCCSSCCCCC1CC1CC)CCN1CCCC1] |
| L0826 | 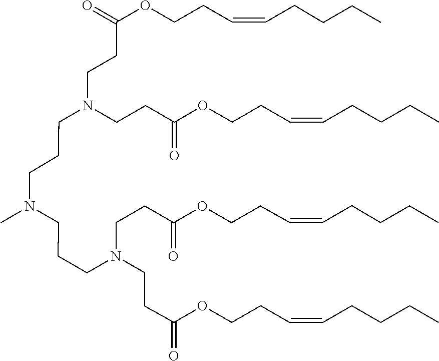 [SMILES: CCCC/C=C\CCOC(=O)CCN(CCCN(C)CCCN(CCC(=O)OCC/C=C\CCCC)CCC(=O)OCC/C=C\CCCC)CCC(=O)OCC/C=C\CCCC] |
| L0828 | 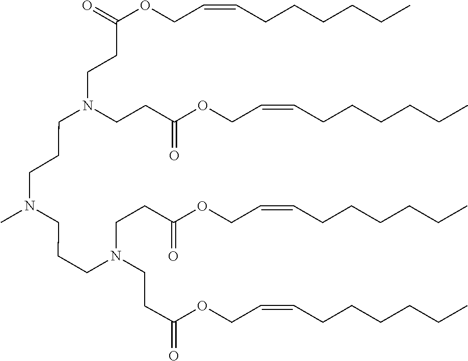 [SMILES: CCCCCC/C=C\COC(=O)CCN(CCCN(C)CCCN(CCC(=O)OC/C=C\CCCCCC)CCC(=O)OC/C=C\CCCCCC)CCC(=O)OC/C=C\CCCCCC] |
| L0531 | 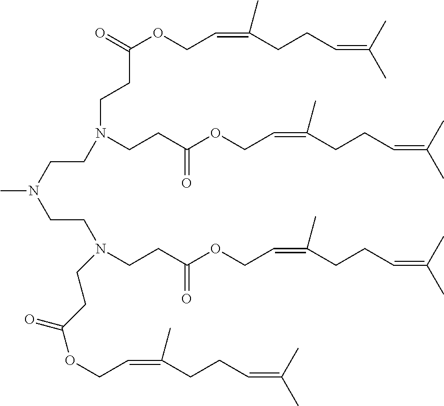 [SMILES: CC(C)=CCC/C(C)=C\COC(=O)CCN(CCC(=O)OC/C=C(/C)CCC=C(C)C)CCN(C)CCN(CCC(=O)OC/C=C(/C)CCC=C(C)C)CCC(=O)OC/C=C(/C)CCC=C(C)C] |
| L0534 | 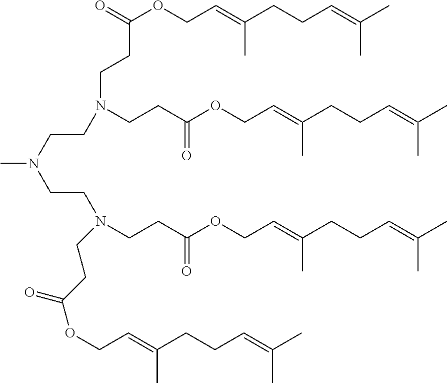 [SMILES: CC(C)=CCC/C(C)=C/COC(=O)CCN(CCC(=O)OC/C=C(\C)CCC=C(C)C)CCN(C)CCN(CCC(=O)OC/C=C(\C)CCC=C(C)C)CCC(=O)OC/C=C(\C)CCC=C(C)C] |
| L0823 | 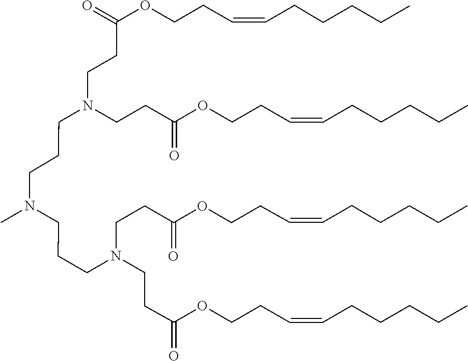 [SMILES: CCCCC/C=C\CCOC(=O)CCN(CCCN(C)CCCN(CCC(=O)OCC/C=C\CCCCC)CCC(=O)OCC/C=C\CCCCC)CCC(=O)OCC/C=C\CCCCC] |
| L0824 | 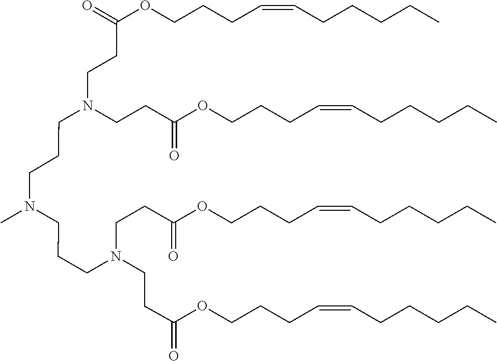 [SMILES: CCCCC/C=C\CCCOC(=O)CCN(CCCN(C)CCCN(CCC(=O)OCCC/C=C\CCCCC)CCC(=O)OCCC/C=C\CCCCC)CCC(=O)OCCC/C=C\CCCCC] |
| L0823 | 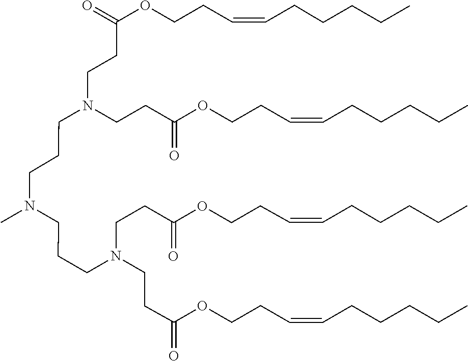 [SMILES: CCCCC/C=C\CCOC(=O)CCN(CCCN(C)CCCN(CCC(=O)OCC/C=C\CCCCC)CCC(=O)OCC/C=C\CCCCC)CCC(=O)OCC/C=C\CCCCC] |
| L0532 | 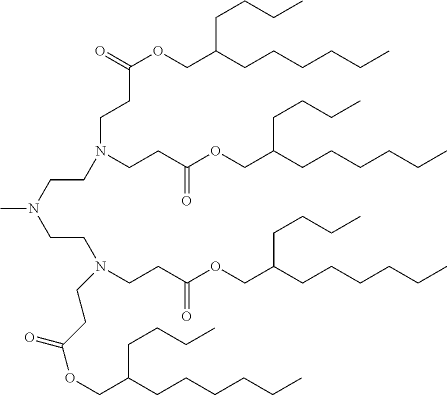 [SMILES: CCCCCCC(CCCC)COC(=O)CCN(CCC(=O)OCC(CCCC)CCCCCC)CCN(C)CCN(CCC(=O)OCC(CCCC)CCCCCC)CCC(=O)OCC(CCCC)CCCCCC] |
| L0533 | 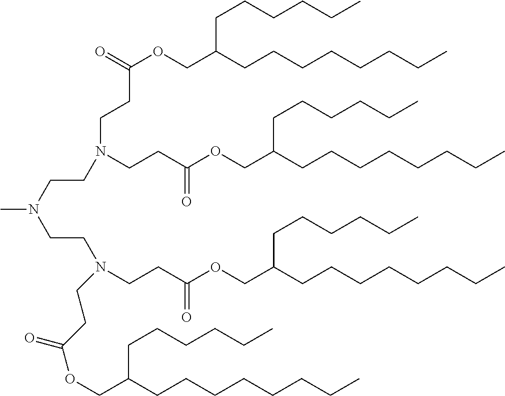 [SMILES: CCCCCCCCC(CCCCCC)COC(=O)CCN(CCC(=O)OCC(CCCCCC)CCCCCCCC)CCN(C)CCN(CCC(=O)OCC(CCCCCC)CCCCCCCC)CCC(=O)OCC(CCCCCC)CCCCCCCC] |
| L0535 | 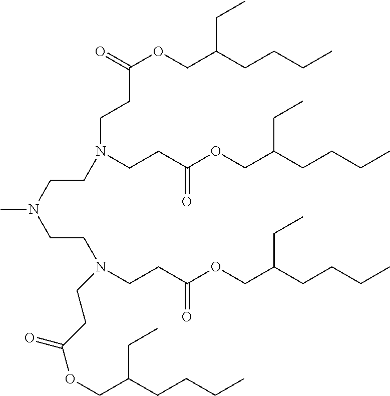 [SMILES: CCCCC(CC)COC(=O)CCN(CCC(=O)OCC(CC)CCCC)CCN(C)CCN(CCC(=O)OCC(CC)CCCC)CCC(=O)OCC(CC)CCCC] |
| L0630 | 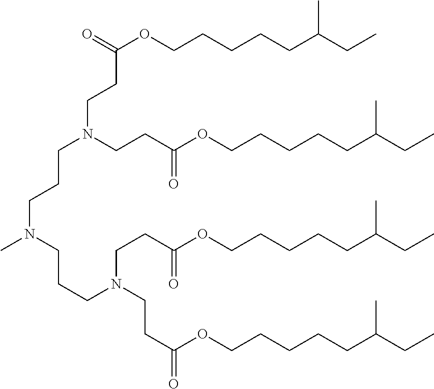 [SMILES: CCC(C)CCCCCOC(=O)CCN(CCCN(C)CCCN(CCC(=O)OCCCCCC(C)CC)CCC(=O)OCCCCCC(C)CC)CCC(=O)OCCCCCC(C)CC] |
| L0631 | 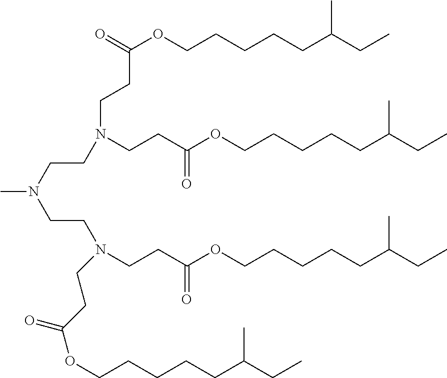 [SMILES: CCC(C)CCCCCOC(=O)CCN(CCC(=O)OCCCCCC(C)CC)CCN(C)CCN(CCC(=O)OCCCCCC(C)CC)CCC(=O)OCCCCCC(C)CC] |
| L0632 | 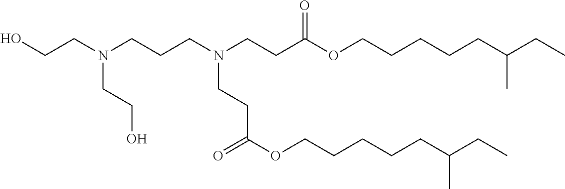 [SMILES: CCC(C)CCCCCOC(=O)CCN(CCCN(CCO)CCO)CCC(=O)OCCCCCC(C)CC] |
| L0633 | 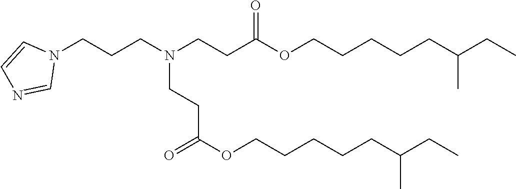 [SMILES: CCC(C)CCCCCOC(=O)CCN(CCCn1ccnc1)CCC(=O)OCCCCCC(C)CC] |
| L0506 | 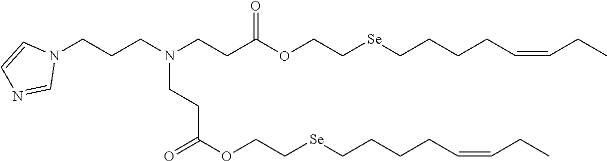 [SMILES: CC/C=C\CCCC[Se]CCOC(=O)CCN(CCCn1ccnc1)CCC(=O)OCC[Se]CCCC/C=C\CC] |
| L0507 | 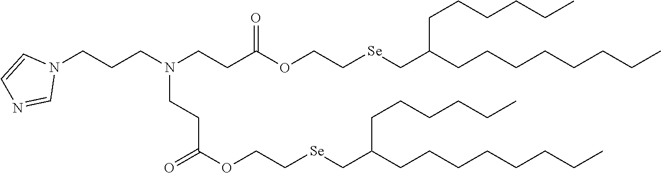 [SMILES: CCCCCCCCC(CCCCCC)C[Se]CCOC(=O)CCN(CCCn1ccnc1)CCC(=O)OCC[Se]CC(CCCCCC)CCCCCCCC] |
| L0508 | 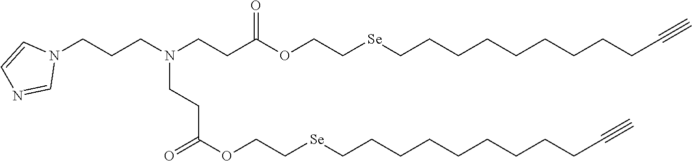 [SMILES: C#CCCCCCCCCC[Se]CCOC(=O)CCN(CCCn1ccnc1)CCC(=O)OCC[Se]CCCCCCCCCC#C] |
| L0509 | 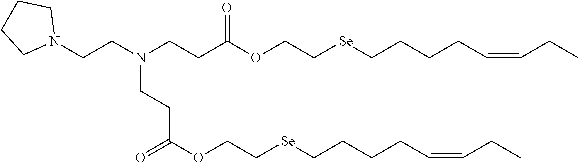 [SMILES: CC/C=C\CCCC[Se]CCOC(=O)CCN(CCC(=O)OCC[Se]CCCC/C=C\CC)CCN1CCCC1] |
| L0510 | 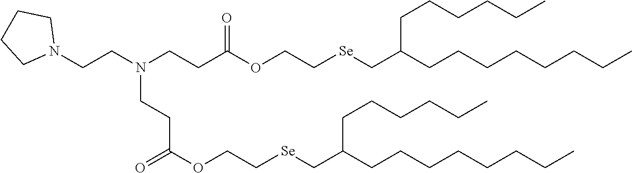 [SMILES: CCCCCCCCC(CCCCCC)C[Se]CCOC(=O)CCN(CCC(=O)OCC[Se]CC(CCCCCC)CCCCCCCC)CCN1CCCC1] |
| L0511 | 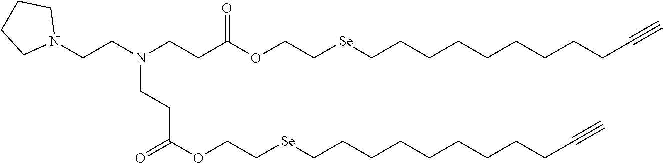 [SMILES: C#CCCCCCCCCC[Se]CCOC(=O)CCN(CCC(=O)OCC[Se]CCCCCCCCCC#C)CCN1CCCC1] |
| L0512 | 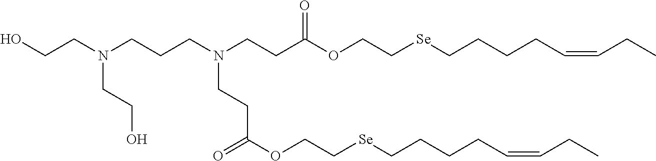 [SMILES: CC/C=C\CCCC[Se]CCOC(=O)CCN(CCCN(CCO)CCO)CCC(=O)OCC[Se]CCCC/C=C\CC] |
| L0513 | 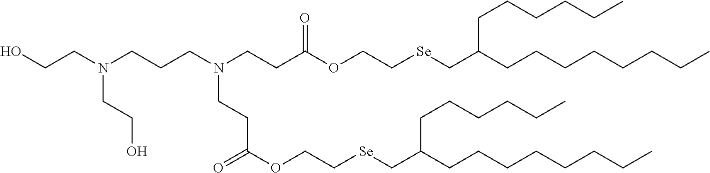 [SMILES: CCCCCCCCC(CCCCCC)C[Se]CCOC(=O)CCN(CCCN(CCO)CCO)CCC(=O)OCC[Se]CC(CCCCCC)CCCCCCCC] |
| L0514 | 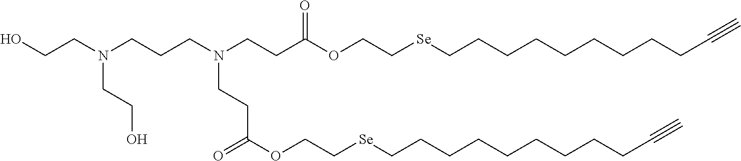 [SMILES: C#CCCCCCCCCC[Se]CCOC(=O)CCN(CCCN(CCO)CCO)CCC(=O)OCC[Se]CCCCCCCCCC#C] |
| L0825 | 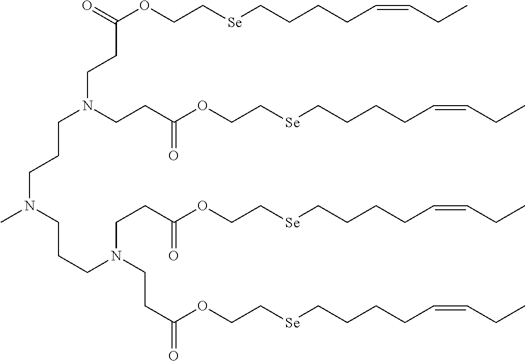 [SMILES: CC/C=C\CCCC[Se]CCOC(=O)CCN(CCCN(C)CCCN(CCC(=O)OCC[Se]CCCC/C=C\CC)CCC(=O)OCC[Se]CCCC/C=C\CC)CCC(=O)OCC[Se]CCCC/C=C\CC] |
| L0580 | 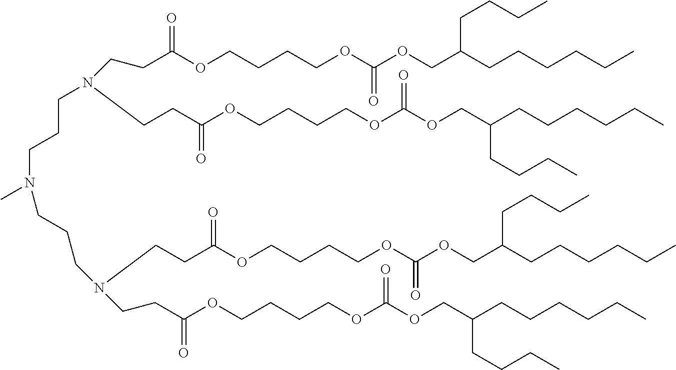 [SMILES: CCCCCCC(CCCC)COC(=O)OCCCCOC(=O)CCN(CCCN(C)CCCN(CCC(=O)OCCCCOC(=O)OCC(CCCC)CCCCCC)CCC(=O)OCCCCOC(=O)OCC(CCCC)CCCCCC)CCC(=O)OCCCCOC(=O)OCC(CCCC)CCCCCC] |
| L0582 | 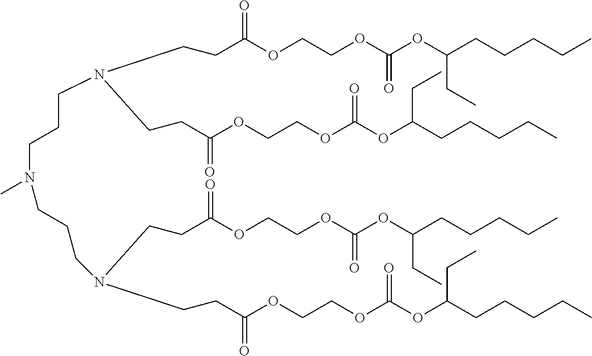 [SMILES: CCCCCC(CC)OC(=O)OCCOC(=O)CCN(CCCN(C)CCCN(CCC(=O)OCCOC(=O)OC(CC)CCCCC)CCC(=O)OCCOC(=O)OC(CC)CCCCC)CCC(=O)OCCOC(=O)OC(CC)CCCCC] |
| L0550 | 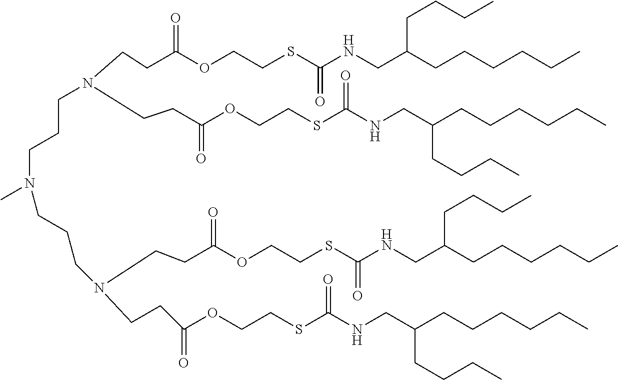 [SMILES: CCCCCCC(CCCC)CNC(=O)SCCOC(=O)CCN(CCCN(C)CCCN(CCC(=O)OCCSC(=O)NCC(CCCC)CCCCCC)CCC(=O)OCCSC(=O)NCC(CCCC)CCCCCC)CCC(=O)OCCSC(=O)NCC(CCCC)CCCCCC] |
| L0551 | 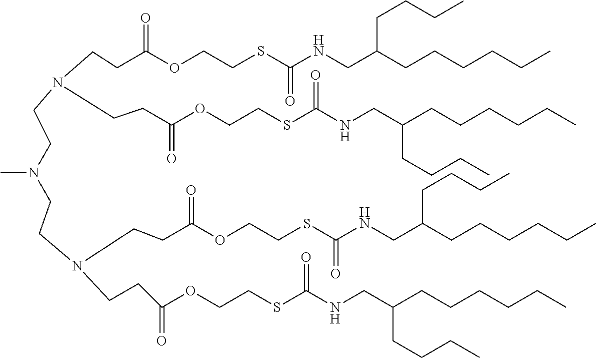 [SMILES: CCCCCCC(CCCC)CNC(=O)SCCOC(=O)CCN(CCC(=O)OCCSC(=O)NCC(CCCC)CCCCCC)CCN(C)CCN(CCC(=O)OCCSC(=O)NCC(CCCC)CCCCCC)CCC(=O)OCCSC(=O)NCC(CCCC)CCCCCC] |
| L0552 | 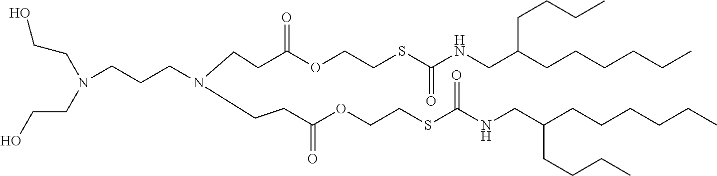 [SMILES: CCCCCCC(CCCC)CNC(=O)SCCOC(=O)CCN(CCCN(CCO)CCO)CCC(=O)OCCSC(=O)NCC(CCCC)CCCCCC] |
| L0553 | 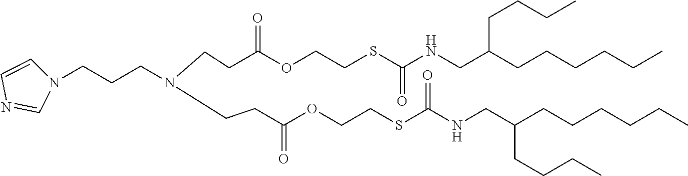 [SMILES: CCCCCCC(CCCC)CNC(=O)SCCOC(=O)CCN(CCCn1ccnc1)CCC(=O)OCCSC(=O)NCC(CCCC)CCCCCC] |
| L0555 | 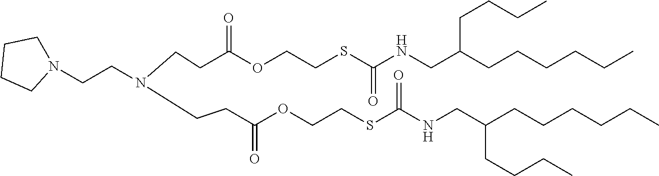 [SMILES: CCCCCCC(CCCC)CNC(=O)SCCOC(=O)CCN(CCC(=O)OCCSC(=O)NCC(CCCC)CCCCCC)CCN1CCCC1] |
| L0637 | 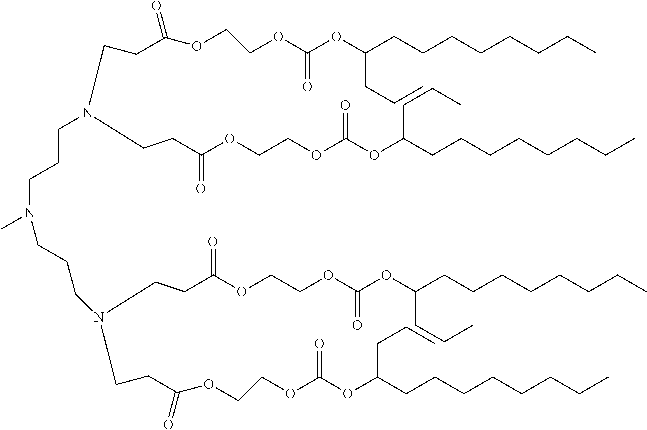 [SMILES: CCCCCCCCC(CCC)OC(=O)OCCOC(=O)CCN(CCCN(C)CCCN(CCC(=O)OCCOC(=O)OC(CCC)CCCCCCCC)CCC(=O)OCCOC(=O)OC(CCC)CCCCCCCC)CCC(=O)OCCOC(=O)OC(CCC)CCCCCCCC] |
| L0638 | 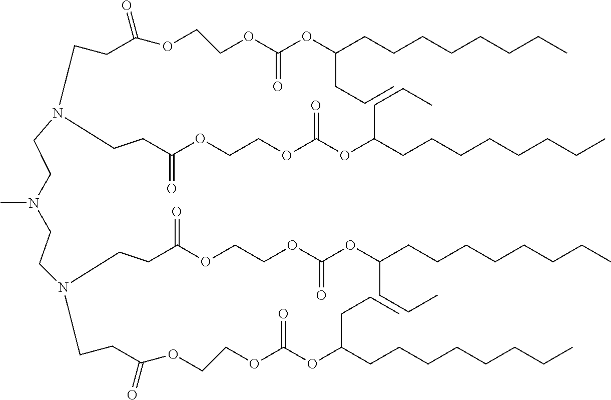 [SMILES: CCCCCCCCC(CCC)OC(=O)OCCOC(=O)CCN(CCC(=O)OCCOC(=O)OC(CCC)CCCCCCCC)CCN(C)CCN(CCC(=O)OCCOC(=O)OC(CCC)CCCCCCCC)CCC(=O)OCCOC(=O)OC(CCC)CCCCCCCC] |
| L0639 | 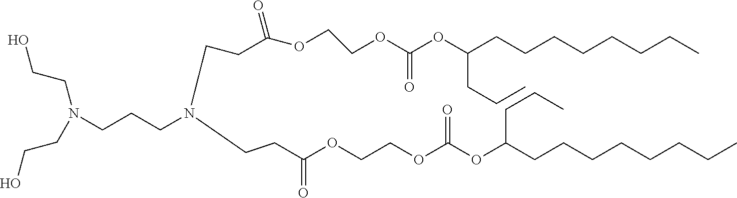 [SMILES: CCCCCCCCC(CCC)OC(=O)OCCOC(=O)CCN(CCCN(CCO)CCO)CCC(=O)OCCOC(=O)OC(CCC)CCCCCCCC] |
| L0640 | 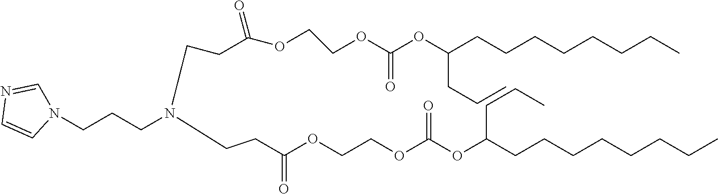 [SMILES: CCCCCCCCC(CCC)OC(=O)OCCOC(=O)CCN(CCCn1ccnc1)CCC(=O)OCCOC(=O)OC(CCC)CCCCCCCC] |
| L0654 | 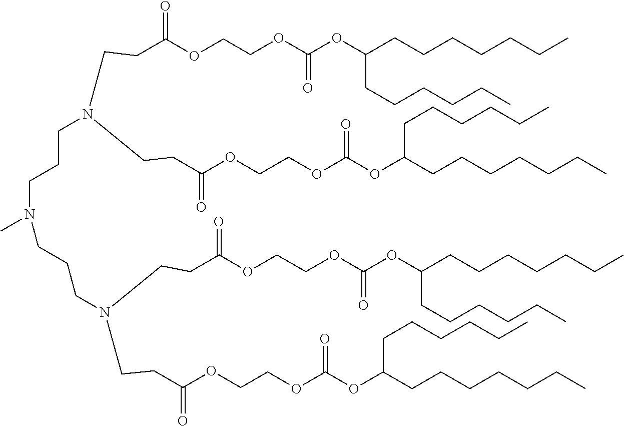 [SMILES: CCCCCCCC(CCCCCC)OC(=O)OCCOC(=O)CCN(CCCN(C)CCCN(CCC(=O)OCCOC(=O)OC(CCCCCC)CCCCCCC)CCC(=O)OCCOC(=O)OC(CCCCCC)CCCCCCC)CCC(=O)OCCOC(=O)OC(CCCCCC)CCCCCCC] |
| L0655 | 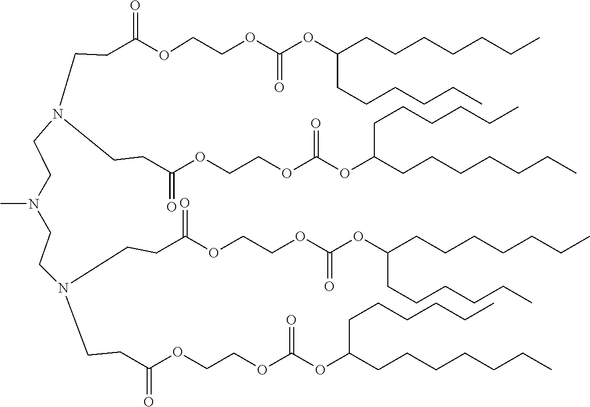 [SMILES: CCCCCCCC(CCCCCC)OC(=O)OCCOC(=O)CCN(CCC(=O)OCCOC(=O)OC(CCCCCC)CCCCCCC)CCN(C)CCN(CCC(=O)OCCOC(=O)OC(CCCCCC)CCCCCCC)CCC(=O)OCCOC(=O)OC(CCCCCC)CCCCCCC] |
| L0656 | 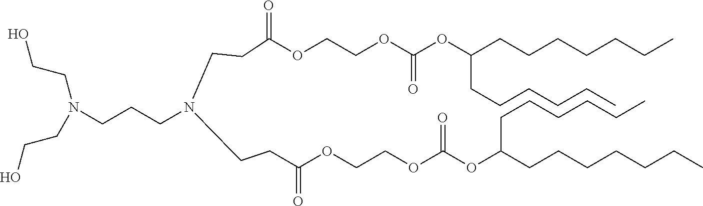 [SMILES: CCCCCCCC(CCCCCC)OC(=O)OCCOC(=O)CCN(CCCN(CCO)CCO)CCC(=O)OCCOC(=O)OC(CCCCCC)CCCCCCC] |
| L0657 | 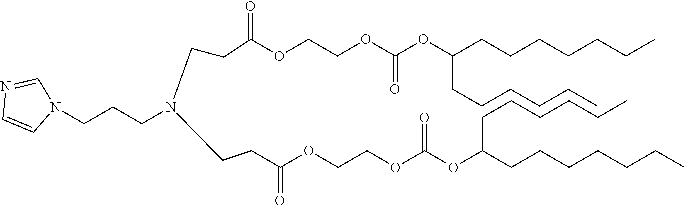 [SMILES: CCCCCCCC(CCCCCC)OC(=O)OCCOC(=O)CCN(CCCn1ccnc1)CCC(=O)OCCOC(=O)OC(CCCCCC)CCCCCCC] |
| L0659 | 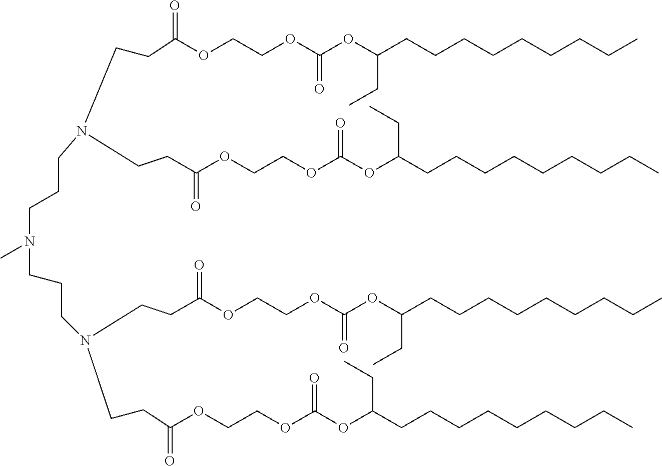 [SMILES: CCCCCCCCCC(CC)OC(=O)OCCOC(=O)CCN(CCCN(C)CCCN(CCC(=O)OCCOC(=O)OC(CC)CCCCCCCCC)CCC(=O)OCCOC(=O)OC(CC)CCCCCCCCC)CCC(=O)OCCOC(=O)OC(CC)CCCCCCCCC] |
| L0660 | 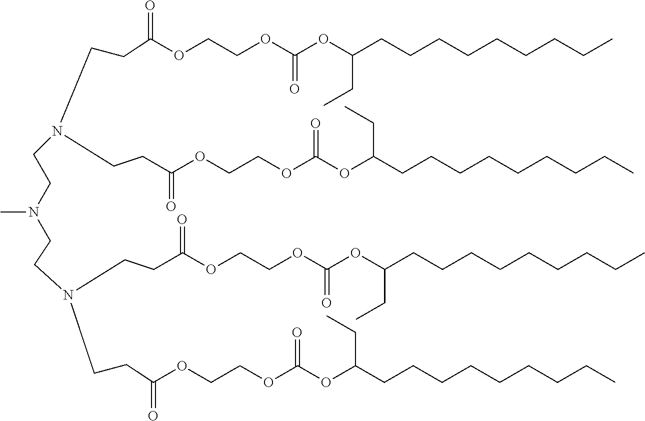 [SMILES: CCCCCCCCCC(CC)OC(=O)OCCOC(=O)CCN(CCC(=O)OCCOC(=O)OC(CC)CCCCCCCCC)CCN(C)CCN(CCC(=O)OCCOC(=O)OC(CC)CCCCCCCCC)CCC(=O)OCCOC(=O)OC(CC)CCCCCCCCC] |
| L0661 | 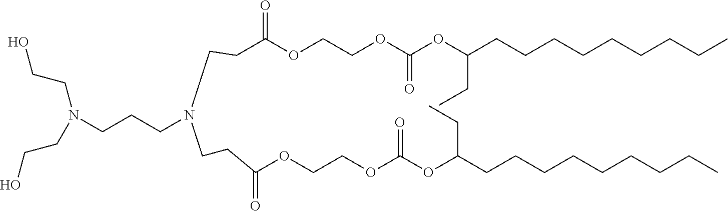 [SMILES: CCCCCCCCCC(CC)OC(=O)OCCOC(=O)CCN(CCCN(CCO)CCO)CCC(=O)OCCOC(=O)OC(CC)CCCCCCCCC] |
| L0662 | 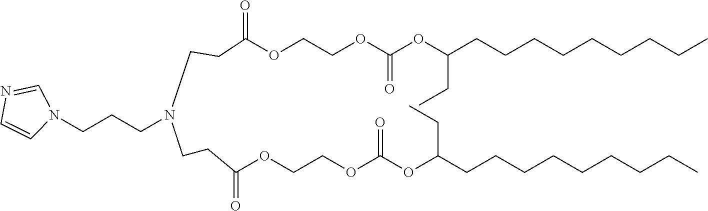 [SMILES: CCCCCCCCCC(CC)OC(=O)OCCOC(=O)CCN(CCCn1ccnc1)CCC(=O)OCCOC(=O)OC(CC)CCCCCCCCC] |
| L0664 | 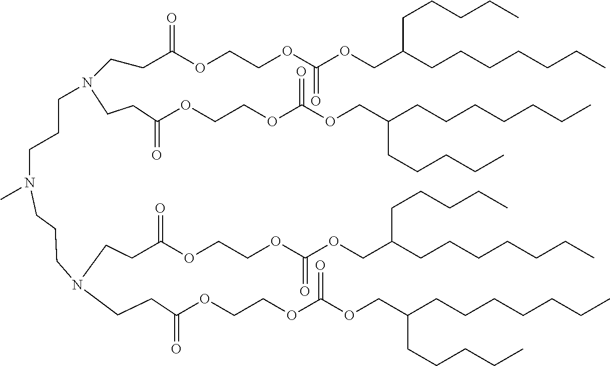 [SMILES: CCCCCCCC(CCCCC)COC(=O)OCCOC(=O)CCN(CCCN(C)CCCN(CCC(=O)OCCOC(=O)OCC(CCCCC)CCCCCCC)CCC(=O)OCCOC(=O)OCC(CCCCC)CCCCCCC)CCC(=O)OCCOC(=O)OCC(CCCCC)CCCCCCC] |
| L0739 | 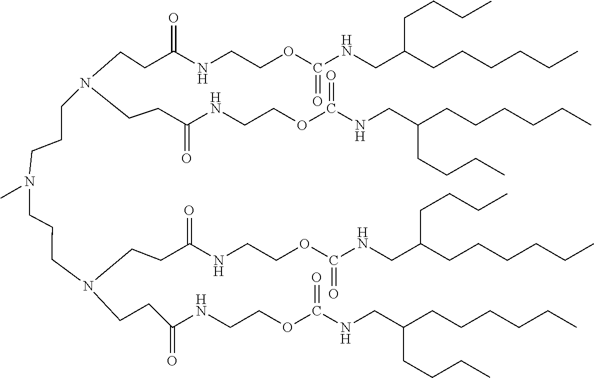 [SMILES: CCCCCCC(CCCC)CNC(=O)OCCNC(=O)CCN(CCCN(C)CCCN(CCC(=O)NCCOC(=O)NCC(CCCC)CCCCCC)CCC(=O)NCCOC(=O)NCC(CCCC)CCCCCC)CCC(=O)NCCOC(=O)NCC(CCCC)CCCCCC] |
| L0740 | 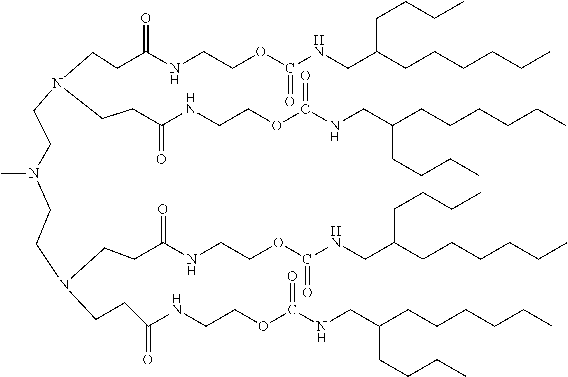 [SMILES: CCCCCCC(CCCC)CNC(=O)OCCNC(=O)CCN(CCC(=O)NCCOC(=O)NCC(CCCC)CCCCCC)CCN(C)CCN(CCC(=O)NCCOC(=O)NCC(CCCC)CCCCCC)CCC(=O)NCCOC(=O)NCC(CCCC)CCCCCC] |
| L0744 | 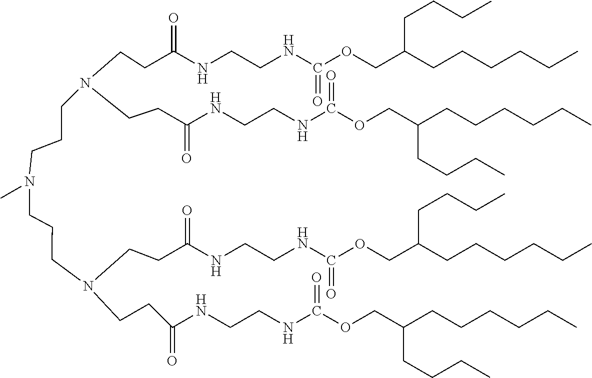 [SMILES: CCCCCCC(CCCC)COC(=O)NCCNC(=O)CCN(CCCN(C)CCCN(CCC(=O)NCCNC(=O)OCC(CCCC)CCCCCC)CCC(=O)NCCNC(=O)OCC(CCCC)CCCCCC)CCC(=O)NCCNC(=O)OCC(CCCC)CCCCCC] |
| L0745 | 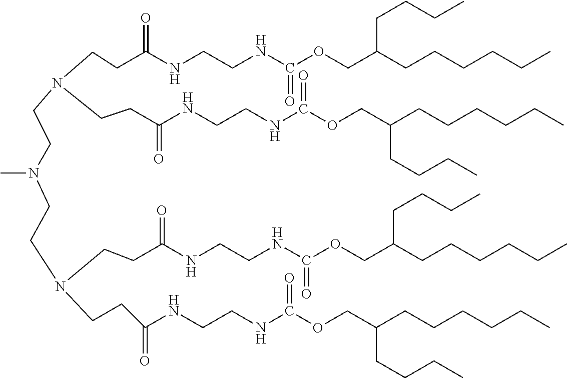 [SMILES: CCCCCCC(CCCC)COC(=O)NCCNC(=O)CCN(CCC(=O)NCCNC(=O)OCC(CCCC)CCCCCC)CCN(C)CCN(CCC(=O)NCCNC(=O)OCC(CCCC)CCCCCC)CCC(=O)NCCNC(=O)OCC(CCCC)CCCCCC] |
| L0775 | 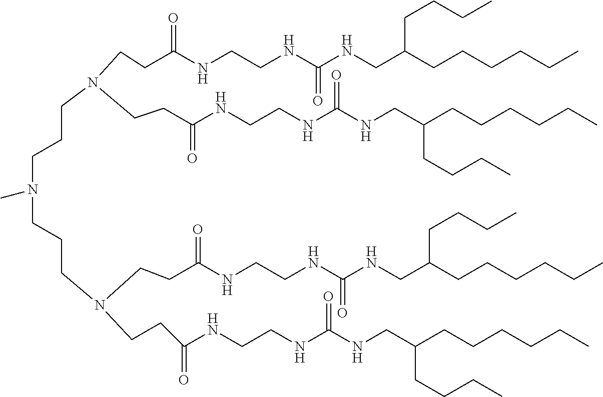 [SMILES: CCCCCCC(CCCC)CNC(=O)NCCNC(=O)CCN(CCCN(C)CCCN(CCC(=O)NCCNC(=O)NCC(CCCC)CCCCCC)CCC(=O)NCCNC(=O)NCC(CCCC)CCCCCC)CCC(=O)NCCNC(=O)NCC(CCCC)CCCCCC] |
| L0776 | 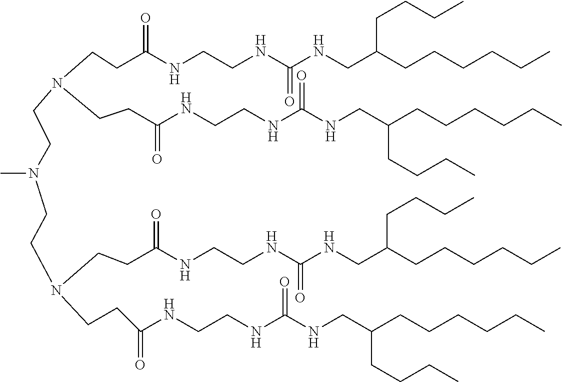 [SMILES: CCCCCCC(CCCC)CNC(=O)NCCNC(=O)CCN(CCC(=O)NCCNC(=O)NCC(CCCC)CCCCCC)CCN(C)CCN(CCC(=O)NCCNC(=O)NCC(CCCC)CCCCCC)CCC(=O)NCCNC(=O)NCC(CCCC)CCCCCC] |
| L0854 | 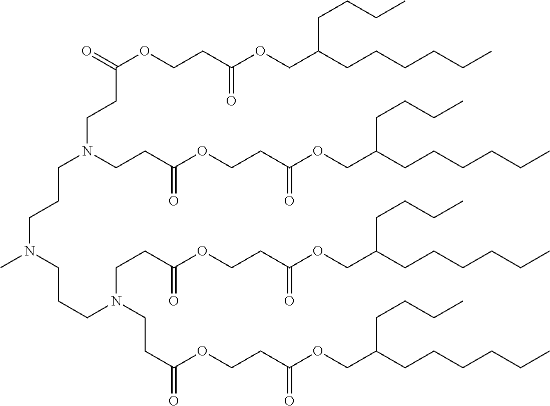 [SMILES: CCCCCCC(CCCC)COC(=O)CCOC(=O)CCN(CCCN(C)CCCN(CCC(=O)OCCC(=O)OCC(CCCC)CCCCCC)CCC(=O)OCCC(=O)OCC(CCCC)CCCCCC)CCC(=O)OCCC(=O)OCC(CCCC)CCCCCC] |
| L0855 | 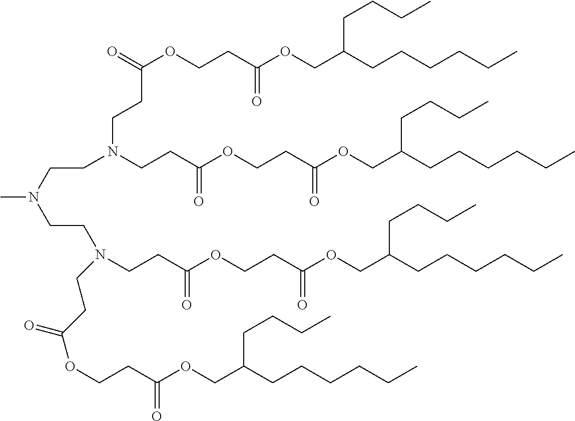 [SMILES: CCCCCCC(CCCC)COC(=O)CCOC(=O)CCN(CCC(=O)OCCC(=O)OCC(CCCC)CCCCCC)CCN(C)CCN(CCC(=O)OCCC(=O)OCC(CCCC)CCCCCC)CCC(=O)OCCC(=O)OCC(CCCC)CCCCCC] |
| L0905 | 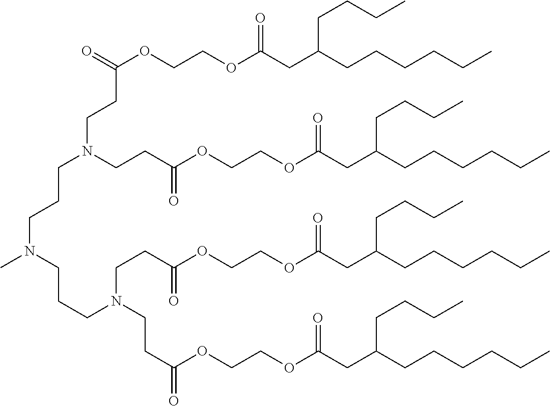 [SMILES: CCCCCCC(CCCC)CC(=O)OCCOC(=O)CCN(CCCN(C)CCCN(CCC(=O)OCCOC(=O)CC(CCCC)CCCCCC)CCC(=O)OCCOC(=O)CC(CCCC)CCCCCC)CCC(=O)OCCOC(=O)CC(CCCC)CCCCCC] |
| L0906 | 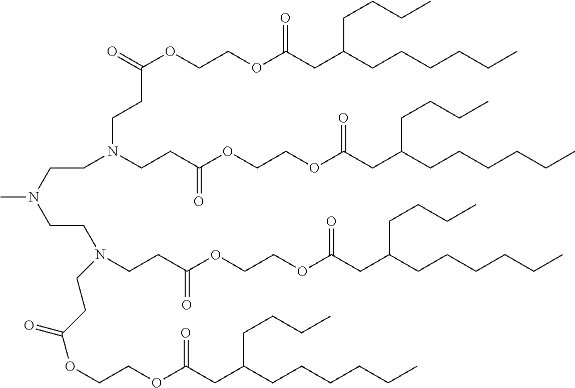 [SMILES: CCCCCCC(CCCC)CC(=O)OCCOC(=O)CCN(CCC(=O)OCCOC(=O)CC(CCCC)CCCCCC)CCN(C)CCN(CCC(=O)OCCOC(=O)CC(CCCC)CCCCCC)CCC(=O)OCCOC(=O)CC(CCCC)CCCCCC] |
| L0888 | 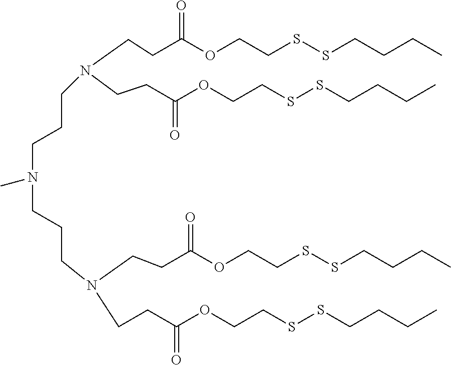 [SMILES: CCCCSSCCOC(=O)CCN(CCCN(C)CCCN(CCC(=O)OCCSSCCCC)CCC(=O)OCCSSCCCC)CCC(=O)OCCSSCCCC] |
| L0889 | 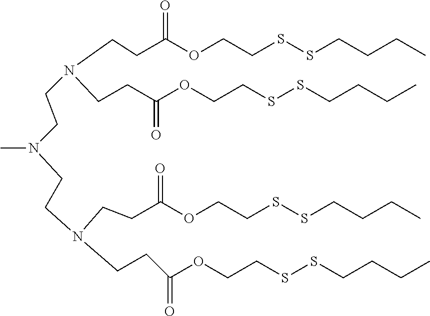 [SMILES: CCCCSSCCOC(=O)CCN(CCC(=O)OCCSSCCCC)CCN(C)CCN(CCC(=O)OCCSSCCCC)CCC(=O)OCCSSCCCC] |
| L0642 | 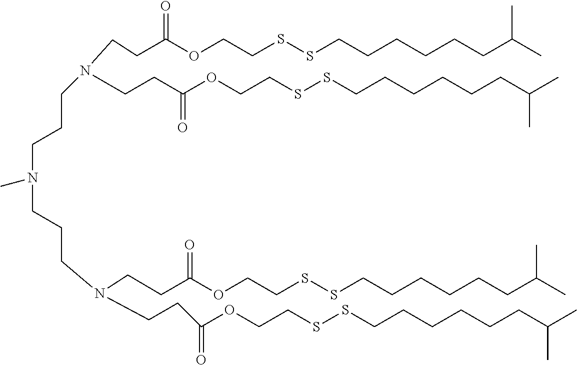 [SMILES: CC(C)CCCCCCSSCCOC(=O)CCN(CCCN(C)CCCN(CCC(=O)OCCSSCCCCCCC(C)C)CCC(=O)OCCSSCCCCCCC(C)C)CCC(=O)OCCSSCCCCCCC(C)C] |
| L0643 | 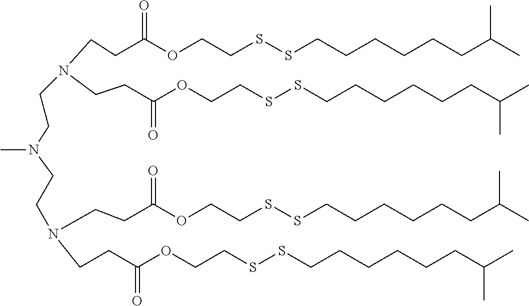 [SMILES: CC(C)CCCCCCSSCCOC(=O)CCN(CCC(=O)OCCSSCCCCCCC(C)C)CCN(C)CCN(CCC(=O)OCCSSCCCCCCC(C)C)CCC(=O)OCCSSCCCCCCC(C)C] |
| L0644 | 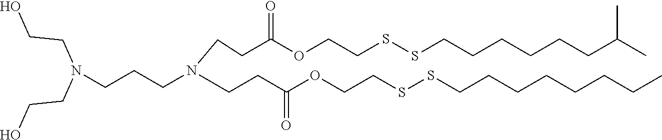 [SMILES: CCCCCCCCSSCCOC(=O)CCN(CCCN(CCO)CCO)CCC(=O)OCCSSCCCCCCC(C)C] |
| L0645 | 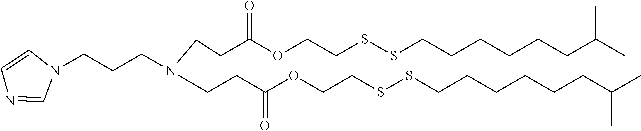 [SMILES: CC(C)CCCCCCSSCCOC(=O)CCN(CCCn1ccnc1)CCC(=O)OCCSSCCCCCCC(C)C] |
| L0893 | 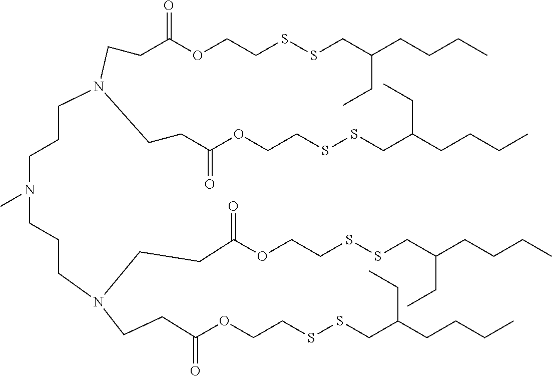 [SMILES: CCCCC(CC)CSSCCOC(=O)CCN(CCCN(C)CCCN(CCC(=O)OCCSSCC(CC)CCCC)CCC(=O)OCCSSCC(CC)CCCC)CCC(=O)OCCSSCC(CC)CCCC] |
| L0894 | 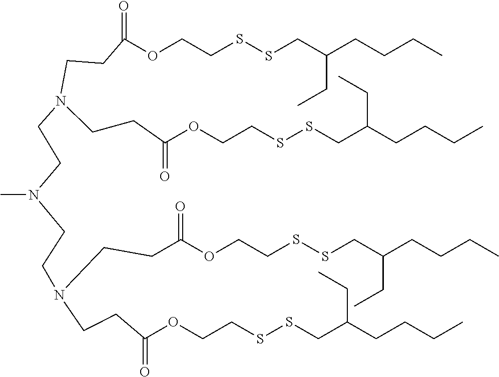 [SMILES: CCCCC(CC)CSSCCOC(=O)CCN(CCC(=O)OCCSSCC(CC)CCCC)CCN(C)CCN(CCC(=O)OCCSSCC(CC)CCCC)CCC(=O)OCCSSCC(CC)CCCC] |
| L0743 | 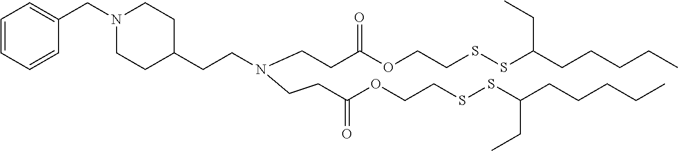 [SMILES: CCCCCC(CC)SSCCOC(=O)CCN(CCC(=O)OCCSSC(CC)CCCCC)CCC1CCN(Cc2ccccc2)CC1] |
| L0882 | 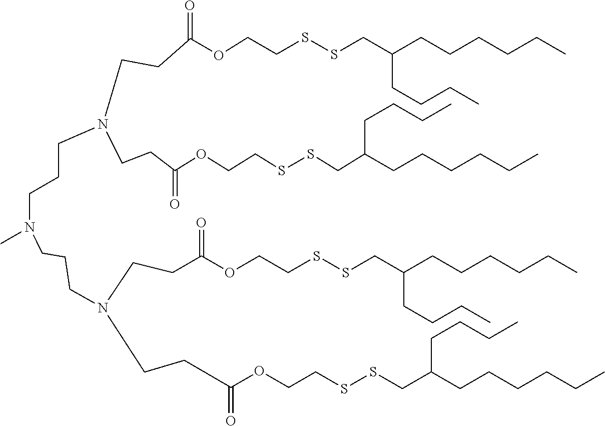 [SMILES: CCCCCCC(CCCC)CSSCCOC(=O)CCN(CCCN(C)CCCN(CCC(=O)OCCSSCC(CCCC)CCCCCC)CCC(=O)OCCSSCC(CCCC)CCCCCC)CCC(=O)OCCSSCC(CCCC)CCCCCC] |
| L0883 | 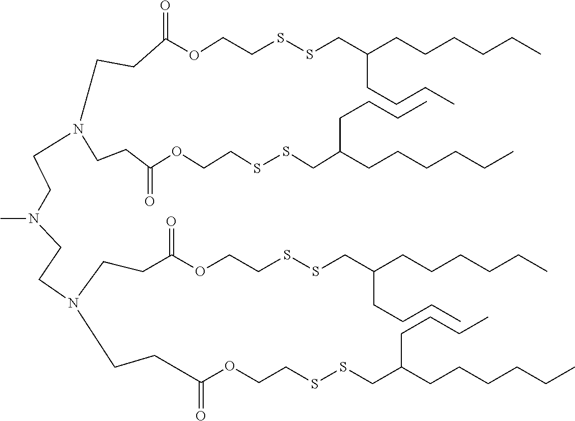 [SMILES: CCCCCCC(CCCC)CSSCCOC(=O)CCN(CCC(=O)OCCSSCC(CCCC)CCCCCC)CCN(C)CCN(CCC(=O)OCCSSCC(CCCC)CCCCCC)CCC(=O)OCCSSCC(CCCC)CCCCCC] |
| L0884 | 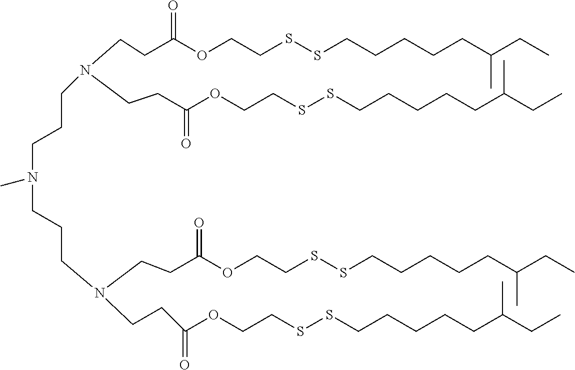 [SMILES: CCC(C)CCCCCSSCCOC(=O)CCN(CCCN(C)CCCN(CCC(=O)OCCSSCCCCCC(C)CC)CCC(=O)OCCSSCCCCCC(C)CC)CCC(=O)OCCSSCCCCCC(C)CC] |
| L0885 | 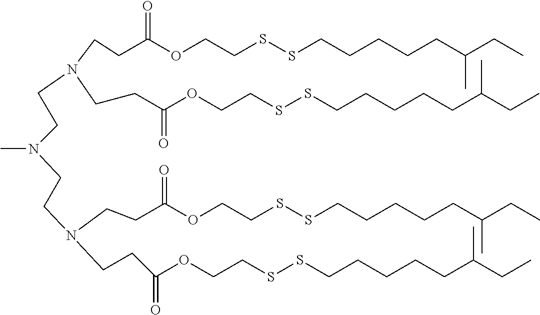 [SMILES: CCC(C)CCCCCSSCCOC(=O)CCN(CCC(=O)OCCSSCCCCCC(C)CC)CCN(C)CCN(CCC(=O)OCCSSCCCCCC(C)CC)CCC(=O)OCCSSCCCCCC(C)CC] |
| L0813 | 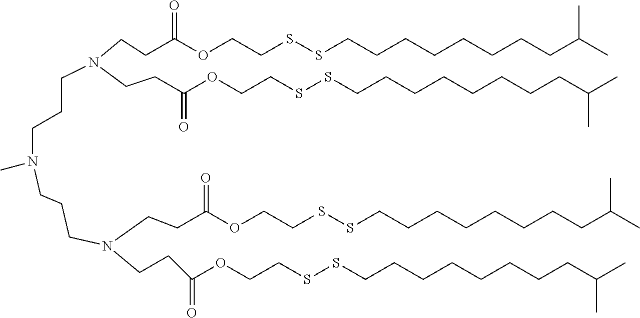 [SMILES: CC(C)CCCCCCCCSSCCOC(=O)CCN(CCCN(C)CCCN(CCC(=O)OCCSSCCCCCCCCC(C)C)CCC(=O)OCCSSCCCCCCCCC(C)C)CCC(=O)OCCSSCCCCCCCCC(C)C] |
| L0814 | 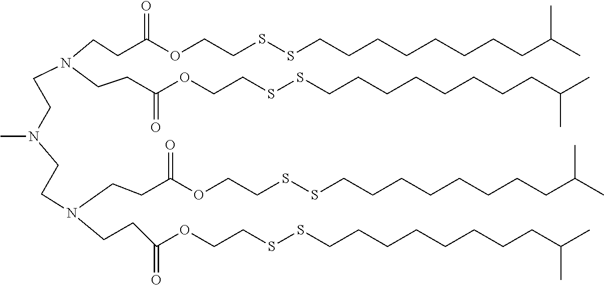 [SMILES: CC(C)CCCCCCCCSSCCOC(=O)CCN(CCC(=O)OCCSSCCCCCCCCC(C)C)CCN(C)CCN(CCC(=O)OCCSSCCCCCCCCC(C)C)CCC(=O)OCCSSCCCCCCCCC(C)C] |
| L0886 | 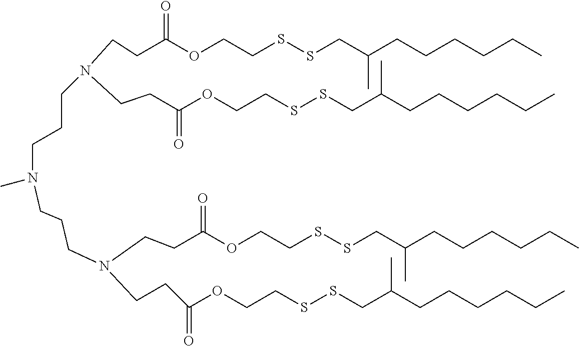 [SMILES: CCCCCCC(C)CSSCCOC(=O)CCN(CCCN(C)CCCN(CCC(=O)OCCSSCC(C)CCCCCC)CCC(=O)OCCSSCC(C)CCCCCC)CCC(=O)OCCSSCC(C)CCCCCC] |
| L0887 | 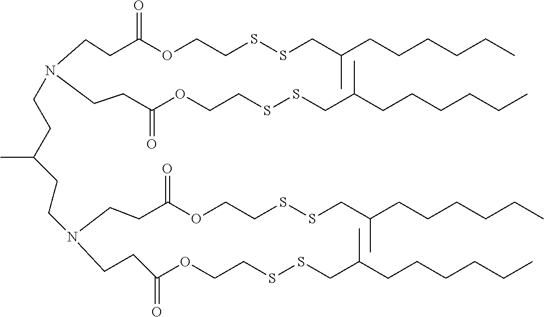 [SMILES: CCCCCCC(C)CSSCCOC(=O)CCN(CCC(=O)OCCSSCC(C)CCCCCC)CCC(C)CCN(CCC(=O)OCCSSCC(C)CCCCCC)CCC(=O)OCCSSCC(C)CCCCCC] |
| L0481 | 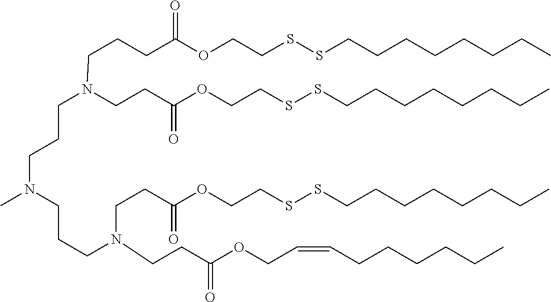 [SMILES: CCCCCC/C=C\COC(=O)CCN(CCCN(C)CCCN(CCCC(=O)OCCSSCCCCCCCC)CCC(=O)OCCSSCCCCCCCC)CCC(=O)OCCSSCCCCCCCC] |
| L0482 | 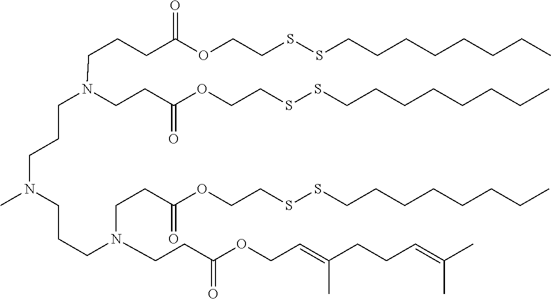 [SMILES: CCCCCCCCSSCCOC(=O)CCCN(CCCN(C)CCCN(CCC(=O)OC/C=C(\C)CCC=C(C)C)CCC(=O)OCCSSCCCCCCCC)CCC(=O)OCCSSCCCCCCCC] |
| L0483 | 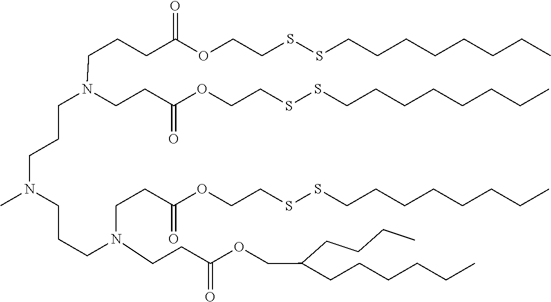 [SMILES: CCCCCCCCSSCCOC(=O)CCCN(CCCN(C)CCCN(CCC(=O)OCCSSCCCCCCCC)CCC(=O)OCC(CCCC)CCCCCC)CCC(=O)OCCSSCCCCCCCC] |
| L0484 | 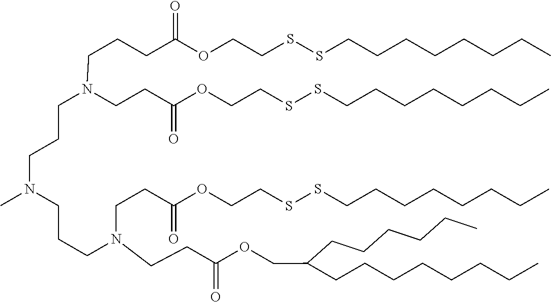 [SMILES: CCCCCCCCSSCCOC(=O)CCCN(CCCN(C)CCCN(CCC(=O)OCCSSCCCCCCCC)CCC(=O)OCC(CCCCCC)CCCCCCCC)CCC(=O)OCCSSCCCCCCCC] |
| L0486 | 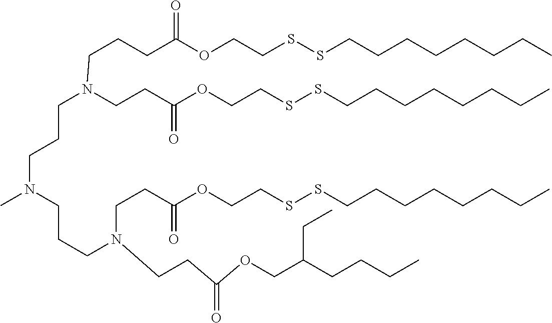 [SMILES: CCCCCCCCSSCCOC(=O)CCCN(CCCN(C)CCCN(CCC(=O)OCCSSCCCCCCCC)CCC(=O)OCC(CC)CCCC)CCC(=O)OCCSSCCCCCCCC] |
| L0581 | 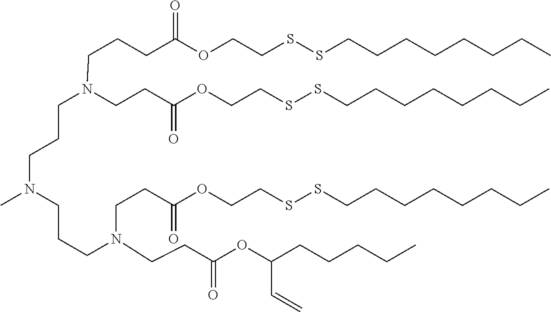 [SMILES: C=CC(CCCCC)OC(=O)CCN(CCCN(C)CCCN(CCCC(=O)OCCSSCCCCCCCC)CCC(=O)OCCSSCCCCCCCC)CCC(=O)OCCSSCCCCCCCC] |
| L0829 | 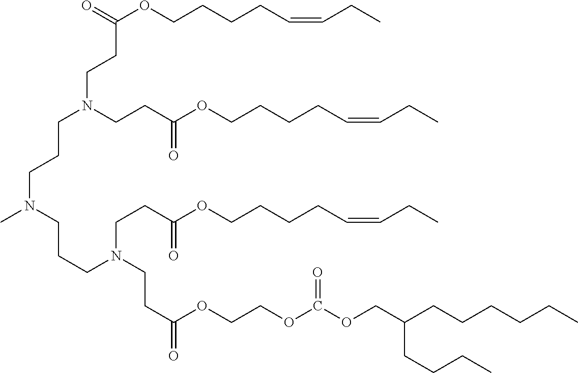 [SMILES: CC/C=C\CCCCOC(=O)CCN(CCCN(C)CCCN(CCC(=O)OCCCC/C=C\CC)CCC(=O)OCCOC(=O)OCC(CCCC)CCCCCC)CCC(=O)OCCCC/C=C\CC] |
| L0830 | 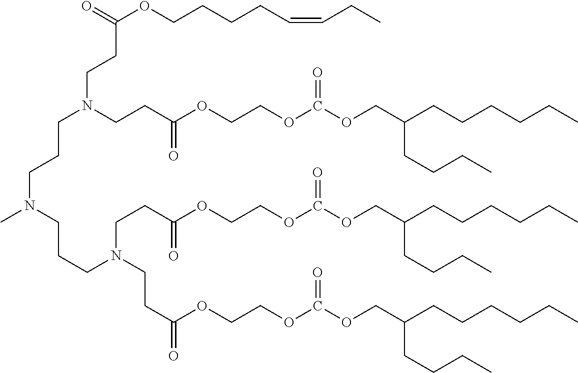 [SMILES: CC/C=C\CCCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| L0836 | 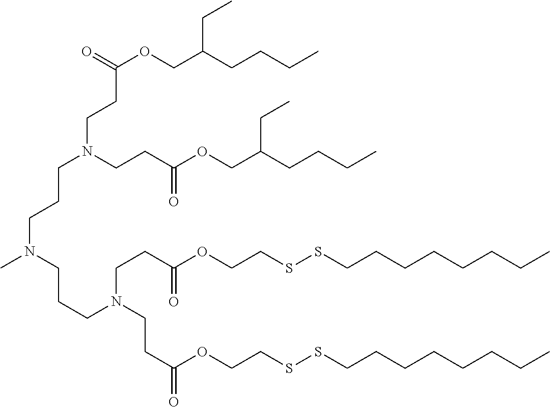 [SMILES: CCCCCCCCSSCCOC(=O)CCN(CCCN(C)CCCN(CCC(=O)OCC(CC)CCCC)CCC(=O)OCC(CC)CCCC)CCC(=O)OCCSSCCCCCCCC] |
| HTX-CH-L0088 | 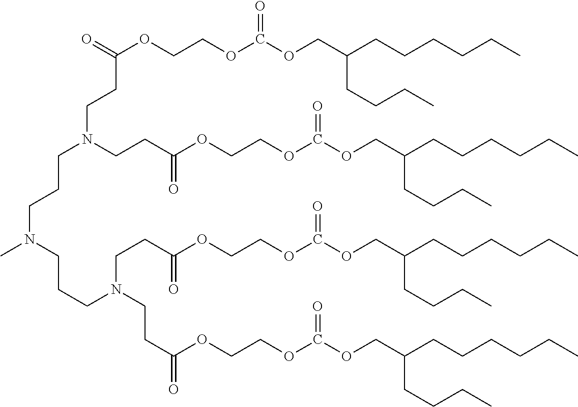 [SMILES: CCCCCCC(CCCC)COC(=O)OCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| HTX-CH-L0093 | 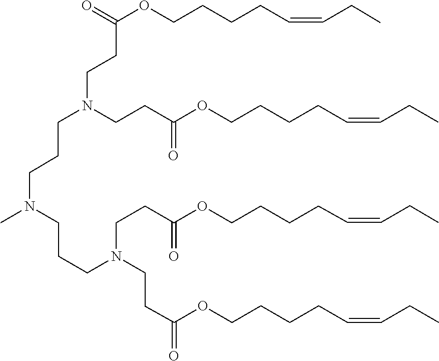 [SMILES: CC/C=C\CCCCOC(=O)CCN(CCCN(C)CCCN(CCC(=O)OCCCC/C=C\CC)CCC(=O)OCCCC/C=C\CC)CCC(=O)OCCCC/C=C\CC] |
| HTX-CH-L0010 | 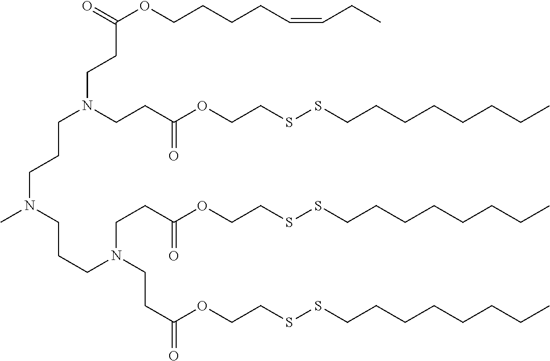 [SMILES: CC/C=C\CCCCOC(=O)CCN(CCCN(C)CCCN(CCC(=O)OCCSSCCCCCCCC)CCC(=O)OCCSSCCCCCCCC)CCC(=O)OCCSSCCCCCCCC] |
| HTX-CH-L0065 | 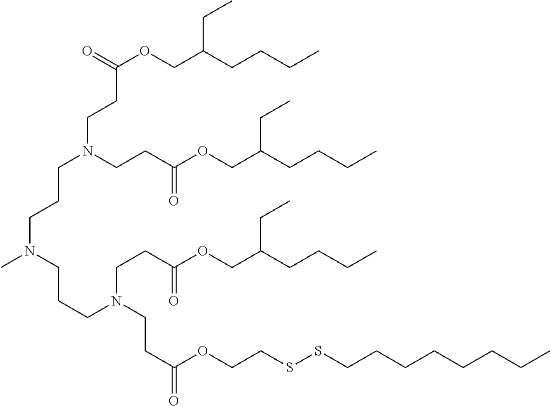 [SMILES: CCCCCCCCSSCCOC(=O)CCN(CCCN(C)CCCN(CCC(=O)OCC(CC)CCCC)CCC(=O)OCC(CC)CCCC)CCC(=O)OCC(CC)CCCC] |
| HTX-CH-L0090 | 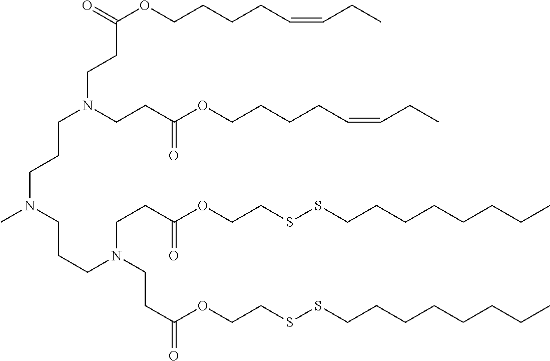 [SMILES: CC/C=C\CCCCOC(=O)CCN(CCCN(C)CCCN(CCC(=O)OCCSSCCCCCCCC)CCC(=O)OCCSSCCCCCCCC)CCC(=O)OCCCC/C=C\CC] |
| HTX-CH-L0095 | 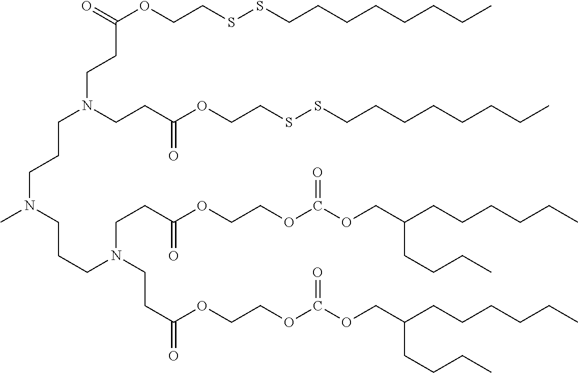 [SMILES: CCCCCCCCSSCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCSSCCCCCCCC] |
| HTX-CH-L0105 | 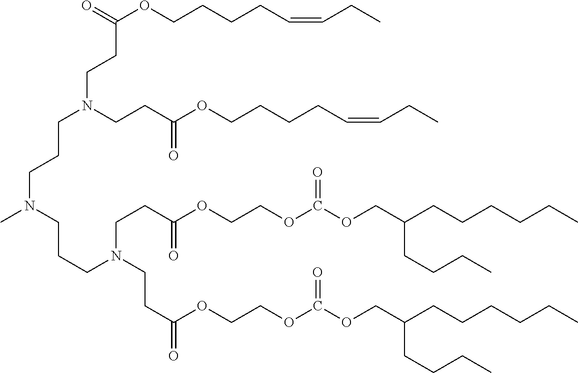 [SMILES: CC/C=C\CCCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCCC/C=C\CC] |
| HTX-CH-L0465 | 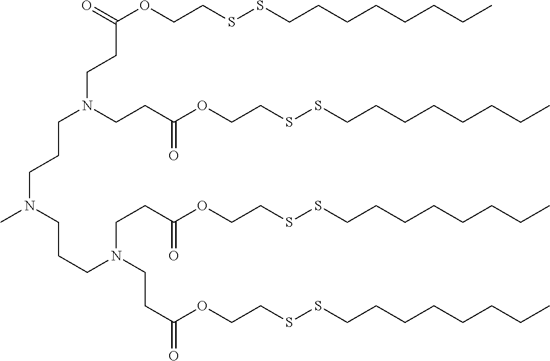 [SMILES: CCCCCCCCSSCCOC(=O)CCN(CCCN(C)CCCN(CCC(=O)OCCSSCCCCCCCC)CCC(=O)OCCSSCCCCCCCC)CCC(=O)OCCSSCCCCCCCC] |

In some embodiments, the composition provided herein further comprises a steroid. In some embodiments, the steroid comprises a cholesterol or a cholesterol derivative. In some embodiments, the composition provided herein further comprises a helper lipid. In some embodiments, the helper lipid comprises a phospholipid, such as 1,2-dioleoyl-sn-glycero-3-phosphoethanolamine (DOPE) or 1,2-dioleoyl-sn-glycero-3-phosphocholine (DOPC). In some embodiments, the composition provided herein further comprises a polymer conjugated lipid. In some embodiments, the polymer conjugated lipid comprises 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k).

In some embodiments, the lipid composition comprises an ionizable lipid disclosed in this application, a steroid, a helper lipid, and a polymer conjugated lipid.

In some embodiments, the ionizable lipid is present in the lipid composition at a weight percentage from about 10% to about 20%, about 10% to about 30%, about 10% to about 40%, about 10% to about 50%, about 10% to about 60%, about 10% to about 80%, about 10% to about 90%, from about 20% to about 30%, from about 20% to about 30%, from about 20% to about 40%, from about 20% to about 50%, from about 20% to about 60%, from about 20% to about 70%, from about 20% to about 80%, from about 20% to about 90%, from about 30% to about 40%, from about 30% to about 50%, from about 30% to about 60%, from about 30% to about 70%, from about 30% to about 80%, from about 30% to about 90%, from about 40% to about 50%, from about 40% to about 60%, from about 40% to about 70%, from about 40% to about 80%, from about 40% to about 90%, from about 50% to about 60%, from about 50% to about 70%, from about 50% to about 80%, from about 50% to about 90%, from about 60% to about 70%, from about 60% to about 80%, from about 60% to about 90%, from about 70% to about 80%, from about 70% to about 90%, or from about 80% to about 90%.

In some embodiments, the helper lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the steroid is present in the lipid composition at a weight percentage from about 10% to about 20%, from about 10% to about 30%, from about 10% to about 40%, from about 20% to about 30%, from about 20% to about 40%, or from about 30% to about 40%.

In some embodiments, the polymer conjugated lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the weight ratio of the ionizable lipid/steroid/helper lipid/polymer conjugated lipid is about 14/4/1/1, about 15/4/1/1, about 16/4/1/1, about 17/4/1/1, about 18/4/1/1, about 19/4/1/1, about 20/4/1/1, about 14/4/2/1, about 15/4/2/1, about 16/4/2/1, about 16.8/4/2/1, about 17/4/2/1, about 18/4/2/1, about 19/4/2/1, or about 20/4/2/1.

In some embodiments, the lipid composition comprises an ionizable lipid disclosed in this application, a steroid and a helper lipid. In some embodiments, the ionizable lipid is present in the lipid composition at a weight percentage from about 30% to about 90%. In some embodiments, the helper lipid is present in the lipid composition at a weight percentage from about 5% to about 40%. In some embodiments, the steroid is present in the lipid composition at a weight percentage from about 5% to about 40%. In some embodiments, the weight ratio of the ionizable lipid/steroid/helper lipid is about 1/1/1, 2/1/1, about 3/1/1, about 4/1/1, about 5/1/1, about 6/1/1, about 2/2/1, about 3/2/1, about 4/2/1, about 5/2/1, or about 6/2/1.

In some embodiments, the lipid composition further comprises a pharmaceutically acceptable carrier. In some embodiments, a carrier comprises a pharmaceutically acceptable excipient. The carrier can comprise a sugar, wherein the sugar comprises mannitol, sucrose, maltose, or trehalose. The carrier can comprise EC-16, (2-hydroxypropyl)-β-cyclodextrin ((HP-β-CD), stearic acid, Perfluoroundecanoic, Saponin, Mannitol, Borneol, Amikacin-EC16, Kanamycin-EC16, Neomycin-EC16, or Bile salts. In some embodiments, the carrier is present in the composition at a weight percentage from about 5% to about 60%. In some embodiments, the excipient is present in the composition at a weight percentage from about 1% to about 70%, from about 5% to about 60%, from about 5% to about 50%, from about 5% to about 40%, from about 5% to about 30%, from about 10% to about 50%, from about 10% to about 40%, from about 10% to about 30%, or from about 10% to about 20%.

In some embodiments, the pharmaceutical composition provided herein may further comprises polynucleotide, an oligonucleotide, a polypeptide, an oligopeptide, a small molecule compound, or any combination thereof. In some embodiments, the polynucleotide is a messenger ribonucleic acid (mRNA).

In some embodiments, the pharmaceutical composition comprises a polynucleotide that encodes a gene product thereof.

In some embodiments, the nucleic acid sequence provided herein is assembled in the lipid composition at a weight ratio of the pharmaceutical agent/lipid composition of from about 1:200 to about 1:100, from about 1:200 to about 1:50, from about 1:200 to about 1:40, from about 1:200 to about 1:30, from about 1:200 to about 1:20, from about 1:200 to about 1:10, from about 1:200 to about 1:5, from about 1:200 to about 1:1, from about 1:100 to about 1:50, from about 1:100 to about 1:40, from about 1:100 to about 1:25, from about 1:100 to about 1:20, from about 1:100 to about 1:15, from about 1:100 to about 1:10, from about 1:100 to about 1:5 or from about 1:100 to about 1:1.

In some embodiments, the composition is formulated for systemic or local administration. In some embodiments, the composition is formulated for intravenous administration. In some embodiments, the composition is formulated for intramuscular administration.

In some embodiments, the lipid nanoparticle targets a tissue or an organ upon administration to a subject. In some embodiments, the tissue or the organ comprises a liver. In some embodiments, the tissue or the organ comprises a lymphoid organ. In some embodiments, the lymphoid organ comprises bone marrow, spleen, or lymph nodes.

In some embodiments, the lipid nanoparticle targets a target cell upon administration to a subject. In some embodiments, the target cell comprises a hepatocyte, a lymphocyte, a leukocyte, a myeloid cell, or a hematopoietic stem cell. In some embodiments, the target cell comprises a B cell, and wherein the B cell comprises a plasmablast, a plasma cell, or a memory B cell. In some embodiments, the target cell comprises a T cell. In some embodiments, the target cell comprises a mature B cell. In some embodiments, the mature B-cell comprises a memory B-cell, a plasma cell, or a plasmablast.

In some embodiments, the target cell comprises a cell in a systemic circulation of the subject. In some embodiments, the target cell comprises a cell within a tissue or an organ of the subject.

In some embodiments, upon administration to a subject the lipid nanoparticle has a faster rate of clearance compared to other compositions.

### Additional Lipids

In some embodiments, the lipid composition further comprises an additional lipid comprising a steroid or a steroid derivative, a PEG lipid, and a helper lipid (e.g., phospholipids or other zwitterionic lipids).

In some embodiments, the lipid composition further comprises a helper lipid. In some embodiments, the helper lipid comprises a lipid that contributes to the stability or delivery efficiency of the lipid compositions. In some embodiments, the helper lipid comprises a zwitterionic lipid. In some embodiments, the helper lipid comprises a phospholipid. In some embodiments, the phospholipid may contain one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge. In some embodiments of the lipid compositions, the phospholipid is not an ethylphosphocholine. In some embodiments, the helper lipid can comprise 1,2-dioleoyl-sn-glycero-3-phosphoethanolamine (DOPE) or 1,2-dioleoyl-sn-glycero-3-phosphocholine (DOPC).

In some embodiments, the compositions may further comprise a molar percentage of the phospholipid to the total lipid composition from about 5 to about 30.

In some embodiments, the helper lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the lipid composition comprises the phospholipid at a molar percentage from about 8% to about 23%. In some embodiments, the lipid composition comprises the phospholipid at a molar percentage from about 10% to about 20%. In some embodiments, the lipid composition comprises the phospholipid at a molar percentage from about 15% to about 20%. In some embodiments, the lipid composition comprises the phospholipid at a molar percentage from about 8% to about 15%. In some embodiments, the lipid composition comprises the phospholipid at a molar percentage from about 10% to about 15%. In some embodiments, the lipid composition comprises the phospholipid at a molar percentage from about 12% to about 18%. In some embodiments, the lipid composition comprises the phospholipid at a molar percentage of at least about 8%, at least about 10%, at least about 12%, at least about 15%, at least about 18%, at least about 20%, or at least about 23%. In some embodiments, the lipid composition comprises the phospholipid at a molar percentage of at most about 8%, at most about 10%, at most about 12%, at most about 15%, at most about 18%, at most about 20%, or at most about 23%.

In some embodiments, the lipid composition further comprises a steroid or steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula:

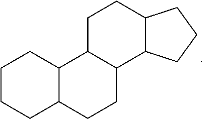 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

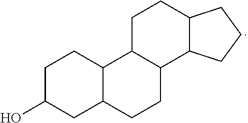 [SMILES: OC1CCC2C(CCC3C4CCCC4CCC23)C1]

In some embodiments, the steroid or steroid derivative is a cholestane or cholestane derivative. In a cholestane, the ring structure is further defined by the formula:

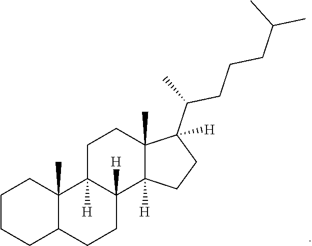

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholesterol and a sterol or a derivative thereof.

In some embodiments, the compositions may further comprise a molar percentage of the steroid to the total lipid composition from about 20 to about 60. In some embodiments, the steroid is present in the lipid composition at a weight percentage from about 10% to about 20%, from about 10% to about 30%, from about 10% to about 40%, from about 20% to about 30%, from about 20% to about 40%, or from about 30% to about 40%.

In some embodiments, the lipid composition comprises the steroid or steroid derivative at a molar percentage from about 15% to about 46%. In some embodiments, the lipid composition comprises the steroid or steroid derivative at a molar percentage from about 20% to about 40%. In some embodiments, the lipid composition comprises the steroid or steroid derivative at a molar percentage from about 25% to about 35%. In some embodiments, the lipid composition comprises the steroid or steroid derivative at a molar percentage from about 30% to about 40%. In some embodiments, the lipid composition comprises the steroid or steroid derivative at a molar percentage from about 20% to about 30%. In some embodiments, the lipid composition comprises the steroid or steroid derivative at a molar percentage of at least about 15%, at least about 20%, at least about 25%, at least about 30%, at least about 35%, at least about 40%, at least about 45%, or at least about 46%. In some embodiments, the lipid composition comprises the steroid or steroid derivative at a molar percentage of at most about 15%, at most about 20%, at most about 25%, at most about 30%, at most about 35%, at most about 40%, at most about 45%, or at most about 46%.

In some embodiments, the lipid composition further comprises a polymer conjugated lipid. In some embodiments, the polymer conjugated lipid is a PEG lipid. In some embodiments, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present application are taught by U.S. Pat. No. 5,820,873, WO 2010/141069, or U.S. Pat. No. 8,450,298, which is incorporated herein by reference.

In some embodiments, the PEG lipid has a structural formula:

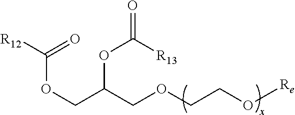

wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤8)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In some embodiments, the PEG lipid has a structural formula:

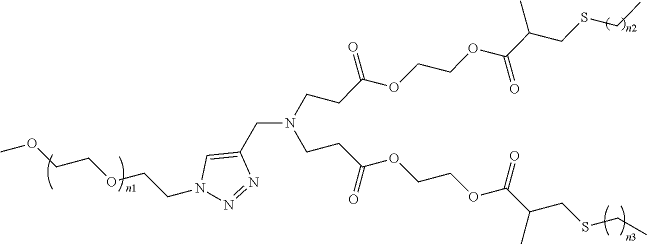

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments, the polymer conjugated lipid comprises 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k).

In some embodiments, the lipid composition comprises the polymer-conjugated lipid at a molar percentage from about 0.5% to about 20%. In some embodiments, the lipid composition comprises the polymer-conjugated lipid at a molar percentage from about 1% to about 8%. In some embodiments, the lipid composition comprises the polymer-conjugated lipid at a molar percentage from about 2% to about 7%. In some embodiments, the lipid composition comprises the polymer-conjugated lipid at a molar percentage from about 3% to about 5%. In some embodiments, the lipid composition comprises the polymer-conjugated lipid at a molar percentage from about 5% to about 10%. In some embodiments, the lipid composition comprises the polymer-conjugated lipid at a molar percentage of at least about 0.5%, at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, or at least about 10%. In some embodiments, the lipid composition comprises the polymer-conjugated lipid at a molar percentage of at most about 0.5%, at most about 1%, at most about 1.5%, at most about 2%, at most about 2.5%, at most about 3%, at most about 3.5%, at most about 4%, at most about 4.5%, at most about 5%, at most about 5.5%, at most about 6%, at most about 6.5%, at most about 7%, at most about 7.5%, at most about 8%, at most about 8.5%, at most about 9%, at most about 9.5%, at most about 10%, at most about 15%, or at most 20%.

In some embodiments, the polymer conjugated lipid is present in the lipid composition at a weight percentage from about 1% to about 5%, from about 1% to about 10%, from about 1% to about 20%, from about 5% to about 10%, from about 5% to about 20%, or from about 10% to about 20%.

### Cancer Immunotherapy

Provided herein are pharmaceutical composition comprising a nucleic acid sequence encapsulated by the lipid composition provided herein, wherein the nucleic acid sequences encoding one or more therapeutic peptides. In some embodiments, the therapeutic peptides can be used for cancer immunotherapy (e.g., B cell malignancy). In some embodiments, the cancer immunotherapy comprises T-cell based therapies. In some embodiments, the one or more therapeutic peptides for cancer immunotherapy comprise an antibody or a fragment thereof.

In some embodiments, the antibody encoded by the mRNA comprises a multi-specific antibody. In some embodiments, the multispecific antibody comprises a bispecific T-cell engager (BiTE). In some embodiments, the BiTE binds to a surface protein on an immune cell (e.g., CD3 on a T-cell) and a tumor antigen. A tumor antigen is a protein derived from a tumor or a cancer cell. A tumor antigen can be over-expressed or uniquely expressed on or in a tumor or a cancer cell, which allows tumor targeting. In some embodiments, the BiTE binds to CD3 and CD19. In some embodiments, the compositions and methods provided herein relate to nucleic acid sequences encoding CD19/CD3 BiTE. In some embodiments, the nucleic acid sequences encoding CD19/CD3 BiTE comprise RNA sequences encoding CD19 BiTE.

In some embodiments, the nucleic acid sequences comprise DNA sequences, which could be translated into RNA sequences encoding CD19 BiTE. In some embodiments, the nucleic acid sequences encoding CD19 BiTE or a portion thereof comprise one or more nucleic acid sequences encoding CD19 BiTE or a portion thereof as listed in TABLE 1. In some embodiments, the nucleic acid sequences encoding CD19 BiTE or a portion thereof further comprise one or more nucleic acid sequences encoding a signal peptide, one or more linker regions, or one or more protein tags.

In some cases, the therapeutic peptides encoded by the mRNA encapsulated by the LNPs of the present disclosure comprise a protein tag (e.g., a 6×His tag). In some cases, the protein tag can facilitate identification or purification of the therapeutic peptide.

In some cases, more than one therapeutic peptide can be encoded by the mRNA encapsulated by the LNPs of the present disclosure. In some cases, the LNPs of the present disclosure comprises mRNA encoding at least 1, 2, 3, 4, 5, 6 or more therapeutic peptides.

In some embodiments, the LNPs comprises mRNAs that encode a multispecific antigen-binding peptide for immunotherapy. In some cases, a multispecific antigen-binding peptide comprises a first domain that binds to an antigen expressed by an immune cell and a second domain that binds to an antigen expressed by a tumor cell. The multispecific antigen-binding peptide can bind to an antigen expressed by any one of the immune cells provided herein.

In some embodiments, the LNPs of the disclosure comprises mRNAs that encode a chimeric antigen receptor derived from a neutralizing antibody comprising an antigen-binding fragment, a transmembrane domain, and an intracellular signaling domain described herein. The term “signaling domain” refers to the functional portion of a protein which acts by transmitting information within the cell to regulate cellular activity via defined signaling pathways by generating second messengers or functioning as effectors by responding to such messengers.

An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines. In some cases, the intracellular signaling domain can comprise a primary intracellular signaling domain. Exemplary primary intracellular signaling domains include those derived from the molecules responsible for primary stimulation, or antigen dependent simulation. In some cases, the intracellular signaling domain can comprise a costimulatory intracellular domain. Exemplary costimulatory intracellular signaling domains include those derived from molecules responsible for costimulatory signals, or antigen independent stimulation. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP 12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that can be used for an efficient immune response. Costimulatory molecules include, but are not limited to, an MHC class I molecule, BTLA and a Toll ligand receptor, as well as 0X40, CD2, CD27, CD28, CD5, ICAM-1, LFA-1 (CD1 1a/CD18) and 4-1BB (CD137).

A costimulatory intracellular signaling domain can be derived from the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), 0X40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like.

The intracellular signaling domain can comprise the entire intracellular portion, or the entire native intracellular signaling domain, of the molecule from which it is derived, or a functional fragment thereof.

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR is used. In some cases, the transmembrane domain can be selected or modified by amino acid substitution to avoid binding of such domains to the transmembrane domains of the same or different surface membrane proteins, e.g., to minimize interactions with other members of the receptor complex. In some cases, the transmembrane domain is capable of homodimerization with another CAR on the CAR T-cell surface. In some cases, the amino acid sequence of the transmembrane domain can be modified or substituted so as to minimize interactions with the binding domains of the native binding partner present in the same CAR T-cell.

The transmembrane domain can be derived either from a natural or from a recombinant source. Where the source is natural, the domain may be derived from any membrane-bound or transmembrane protein. In some cases, the transmembrane domain is capable of signaling to the intracellular domain(s) whenever the CAR has bound to a target. In some cases, the transmembrane domain can include at least the transmembrane region(s) of e.g., the alpha, beta, or zeta chain of the T-cell receptor, CD28, CD3 epsilon, CD45, CD4, CD5, CD8, CD9, CD16, CD22, CD33, CD37, CD64, CD80, CD86, CD134, CD137, CD154.

In some cases, the transmembrane domain can be attached to the extracellular region of the CAR, e.g., the antigen-binding domain of the CAR, via a hinge or a spacer, e.g., a hinge from a human protein. In some cases, the hinge can be a human Ig (immunoglobulin) hinge, e.g., an IgG4 hinge, or a CD8a hinge. In one aspect, the hinge or spacer comprises an IgG4 hinge.

The cytoplasmic domain or region of the CAR includes an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. The term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the present disclosure include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal can also be involved. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic signaling domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use herein include those of TCR zeta, FcR gamma, FcR beta, CD3 gamma, CD3 delta, CD3 epsilon, CD5, CD22, CD79a, CD79b, and CD66d. In some cases, a CAR comprises an intracellular signaling domain, e.g., a primary signaling domain, of CD3-zeta. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

A costimulatory molecule is a cell surface molecule other than an antigen receptor or its ligands that can play a role for an efficient response of lymphocytes to an antigen. Examples of such molecules include CD27, CD28, 4-1BB (CD137), 0X40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706).

A short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In some cases, the two or more, e.g., 2, 3, 4, 5, or more, costimulatory signaling domains, are separated by a linker molecule, e.g., a linker molecule described herein. In some cases, the intracellular signaling domain comprises two costimulatory signaling domains. In some cases, the linker molecule is a glycine residue. In some embodiments, the linker is an alanine residue.

In some embodiments, the mRNA-encoded therapeutic peptides for cancer immunotherapy comprises an immunomodulator. In some cases, an immunomodulator comprises a cytokine, cytokine receptor, chemokine, chemokine receptor, immune co-receptor, or immune co-receptor ligand. Expression of the immunomodulator can be driven by an expression regulatory region disclosed herein.

In some cases, the therapeutic peptides for cancer immunotherapy comprises a cytokine or a functional fragment thereof, for example, G-CSF, GITRL, GM-CSF, IFN-α, IFN-β, IFN-γ, IL-1RA, IL-1α, IL-1β, IL-2, IL-3, IL-4, IL-5, IL-6, IL-7, IL-9, IL-10, IL-11, IL-12, IL-13, IL-14, IL-15, IL-16, IL-17, IL-18, IL-20, IL-23, LIF, LIGHT, LT-3, M-CSF, MSP, OSM, OX40L, SCF, TALL-1, TGF-β, TGF-β1, TGF-β2, TGF-β3, TNF-α, TNF-β, TRAIL, TRANCE, or TWEAK.

In some cases, the therapeutic peptides for cancer immunotherapy comprises a cytokine receptor or a functional fragment thereof, for example, a common gamma chain receptor, a common beta chain receptor, an interferon receptor, a TNF family receptor, a TGF-B receptor, Apo3, CD114, CD115, CD116, CD117, CD118, CD120, CD120a, CD120b, CD121, CD121a, CD121b, CD122, CD123, CD124, CD126, CD127, CD130, CD131, CD132, CD212, CD213, CD213a1, CD213a13, CD213a2, CD25, CD27, CD30, CD4, CD40, CD95 (Fas), CDw119, CDw121b, CDw125, CDw131, CDw136, CDw137 (41BB), CDw210, CDw217, GITR, HVEM, IL-11R, IL-11Ra, IL-14R, IL-15R, IL-15Ra, IL-18R, IL-18Rα, IL-18Rβ, IL-20R, IL-20Rα, IL-20Rβ, IL-9R, LIFR, LTβR, OPG, OSMR, OX40, RANK, TACI, TGF-βR1, TGF-βR2, TGF-β3, TRAILR1, TRAILR2, TRAILR3, or TRAILR4.

In some cases, the therapeutic peptides for cancer immunotherapy comprises a chemokine or a functional fragment thereof, for example, ACT-2, AMAC-a, ATAC, ATAC, BLC, CCL1, CCL11, CCL13, CCL14, CCL15, CCL16, CCL17, CCL18, CCL19, CCL2, CCL20, CCL21, CCL22, CCL23, CCL24, CCL25, CCL26, CCL27, CCL3, CCL4, CCL5, CCL7, CCL8, CKb-6, CKb-8, CTACK, CX3CL1, CXCL1, CXCL10, CXCL11, CXCL12, CXCL13, CXCL14, CXCL2, CXCL3, CXCL4, CXCL5, CXCL6, CXCL7, CXCL8, CXCL9, DC-CK1, ELC, ENA-78, eotaxin, eotaxin-2, eotaxin-3, Eskine, exodus-1, exodus-2, exodus-3, fractalkine, GCP-2, GROa, GROb, GROg, HCC-1, HCC-2, HCC-4, I-309, IL-8, ILC, IP-10, I-TAC, LAG-1, LARC, LCC-1, LD78α, LEC, Lkn-1, LMC, lymphoactin, lymphoactin b, MCAF, MCP-1, MCP-2, MCP-3, MCP-4, MDC, MDNCF, MGSA-a, MGSA-b, MGSA-g, Mig, MIP-1d, MIP-1α, MIP-1β, MIP-2a, MIP-2b, MIP-3, MIP-3α, MIP-3β, MIP-4, MIP-4a, MIP-5, MPIF-1, MPIF-2, NAF, NAP-1, NAP-2, oncostatin, PARC, PF4, PPBP, RANTES, SCM-1a, SCM-1b, SDF-1α/β, SLC, STCP-1, TARC, TECK, XCL1, or XCL2.

In some cases, the therapeutic peptides for cancer immunotherapy comprises a chemokine receptor or a functional fragment thereof, for example, CCR1, CCR2, CCR3, CCR4, CCR5, CCR6, CCR7, CCR8, CCR9, CCR10, CX3CR1, CXCR1, CXCR2, CXCR3, CXCR4, CXCR5, XCR1, or XCR1.

In some cases, the therapeutic peptides for cancer immunotherapy comprises costimulatory immune receptor, or a functional fragment thereof, for example, CD28, 2B4 (CD244, SLAMF4), 4-1BB (CD137), CD2 (LFA2, OX34), CD21, CD226 (DNAM1), CD27 (TNFRSF7), CD30 (TNFRSF8), CD4, CD40, CD8, CD84 (SLAMF5), CRACC (CD319,BLAME), CRTAM (CD355), DcR3, DR3 (TNFRSF25), GITR (CD357), HVEM (CD270), ICOS (CD278), LIGHT, LTOR (TNFRSF3), Ly108 (NTBA,CD352,SLAMF6), Ly9 (CD229,SLAMF3), OX40 (CD134), SLAM (CD150,SLAMF1), TIM1 (HAVCR1,KIM1), or TIM2.

In some cases, the therapeutic peptides for cancer immunotherapy comprise an activating NK receptor, or a functional fragment thereof, for example, CD100 (SEMA4D), CD16 (FcgRIIIA), CD160 (BY55), CD244 (2B4, SLAMF4), CD27, CD94− NKG2C, CD94− NKG2E, CD94−NKG2H, CD96, CRTAM, DAP12, DNAM1 (CD226), KIR2DL4, KIR2DS1, KIR2DS2, KIR2DS3, KIR2DS4, KIR2DS5, KIR3DS1, Ly49, NCR, NKG2D (KLRK1, CD314), NKp30 (NCR3), NKp44 (NCR2), NKp46 (NCR1), NKp80 (KLRF1, CLEC5C), NTB-A (SLAMF6), PSGL1, or SLAMF7 (CRACC, CS1, CD319).

In some cases, the therapeutic peptides for cancer immunotherapy comprise an Fcγ receptor (FcγR), an Fcε receptor (FcεR), an Fcα receptor (FcαR), an Fcμ receptor (FcμR), neonatal Fc receptor (FcRn), CD4, CD5, CD8, CD21, CD22, CD27, CD28, CD32, CD40, CD45, CD66d, CD79a, CD79b, CD80, CD86, CD278 (ICOS), CD247ζ, CD247η, 41BB, DAP10, DAP12, FYN, LAT, Lck, MAPK, MHC complex, NFAT, NF-κB, PLC-γ, iC3b, C3dg, C3d, Zap70, MyD88, a functional fragment thereof, or a combination thereof.

In some cases, the therapeutic peptides for cancer immunotherapy comprise a domain that is, is derived from, interacts with, increases expression of, or activates a transcription factor, such as, for example, E2A, Pax5, EBF, PU.1, Ikaros, GATA3, Th-POK, Tbet, Bcl6, NF-κB, NFAT, AP-1, NFAT, STAT1, STAT2, STAT3, STAT4, STAT5, STAT5A, STAT5B, STAT6, STAT7, IRF1, IRF2, IRF3, IRF4, IRF5, IRF6, IRF7, IRF8, IRF9, AP-1, Eomes, FoxP3, Id2, PLZF, ROR-gamma-T, TCF7, ThPOK, or any combination thereof.

### Methods

In some aspects, provided herein are methods for depleting B cells comprising administering a pharmaceutical composition provided herein to a subject in need thereof. B cell have been found to participate in the etiology of many disorders. It is well known that B cells can produce antibodies against pathogens, a process that is heavily regulated by the immune system. Disorders involving dysregulation of maturation, differentiation and proliferation of B cells may be treated with B cell depletion therapy (BCDT). In some embodiments, the subject in need thereof can have a disorder that requires a BCDT. In some embodiments, the methods for depleting B cells can be used in BCDT. In some embodiments, the methods for depleting B cells can be part of the BCDT.

In some embodiments, BCDT comprises a B-cell malignancy or an autoimmune disorder. In some embodiments, the B-cell malignancy comprises B-cell lymphoma, wherein the B-cell lymphoma comprises diffuse large B-cell lymphoma (DLBCL), follicular lymphoma, chronic lymphocytic leukemia (CLL), small lymphocytic lymphoma (SLL), Mantle cell lymphoma (MCL), Marginal zone lymphomas, Burkitt lymphoma, Lymphoplasmacytic lymphoma (Waldenstrom macroglobulinemia), Hairy cell leukemia, primary central nervous system (CNS) lymphoma, or primary intraocular lymphoma (lymphoma of the eye). In some embodiments, the B-cell lymphoma affects the spleen or lymph nodes.

B cell malignancies can be found not only in circulation, but also in local tissues, such as in the bone marrow or secondary lymphoid tissues. In some embodiments, the B-cell malignancy comprises multiple myeloma.

In some embodiments, BCDT comprises the autoimmune disorder. In some embodiments, the autoimmune disorder comprises allergic disorders, systemic lupus erythematosus (SLE), rheumatoid arthritis (RA), multiple sclerosis (MS), anti-myelin-oligodendrocyte glycoprotein (anti-MOG) spectrum disorders, Neuromyelitis Optica Spectrum Disorder (NMOSD), anti-NMDAR encephalitis, or myasthenia gravis.

In some embodiments, the disorder that requires a B cell depletion therapy (BCDT) further comprises pemphigus vulgaris or Sjogren Syndrome.

In some aspects, provided herein are methods for treating a blood malignancy, comprising administering a pharmaceutical composition provided herein to a subject in need thereof, wherein the subject in need thereof has a blood malignancy.

In some embodiments, the blood malignancy comprises a B-cell lymphoma. In some embodiments, the B-cell lymphoma comprises diffuse large B-cell lymphoma (DLBCL), follicular lymphoma, chronic lymphocytic leukemia (CLL), small lymphocytic lymphoma (SLL), Mantle cell lymphoma (MCL), Marginal zone lymphomas, or Burkitt lymphoma.

Administering as provided herein can comprises administering topically, orally, or by an injection. In some embodiments, the administering comprises administering via an intravenous injection or via an intramuscular injection.

In some embodiments, the pharmaceutical composition provided herein can be used for an approved or off-label indication of an FDA-approved drug, wherein the FDA-approved drug binds to a B-cell antigen. In some embodiments, the pharmaceutical composition provided herein can be used for an approved or off-label indication of blinatumomab, inebilizumab, loncastuximab, or tafasitamab.

In some embodiments, the methods provided herein result in the expression of a therapeutic peptide that has a longer half-life compared to any other method comprising administering an equivalent dose of the therapeutic peptide.

In some embodiments, the method results in the expression of a therapeutic peptide that has a half-life of at least 2 hours, at least 2.5 hours, at least 3 hours, at least 3.5 hours, at least 4 hours, at least 4.5 hours, at least 5 hours, at least 5.5 hours, at least 6 hours, at least 6.5 hours, at least 7 hours, at least 7.5 hours, at least 8 hours, at least 8.5 hours, at least 9 hours, at least 9.5 hours, at least 10 hours, at least 12 hours, at least 16 hours, at least 18 hours, at least 24 hours, at least 1.5 days, at least 2 days, at least 2.5 days, at least 3 days, at least 3.5 days, at least 4 days, at least 4.5 days, at least 5 days, at least 5.5 days, at least 6 days, at least 6.5 days, at least 7 days, at least 7.5 days, at least 8 days, at least 8.5 days, at least 9 days, at least 9.5 days, at least 10 days, at least 11 days, at least 12 days, at least 13 days, at least 14 days, or at least 15 or more days.

In some embodiments, the method results in the expression of a therapeutic peptide that has a larger area under curve (AUC) compared to other methods.

In some embodiments, the method results in the expression of a therapeutic peptide that has an AUC that is at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, or 500% or more larger compared to any other method comprising administering an equivalent dose of the therapeutic peptide.

In some embodiments, the method results in prolonged B-cell depletion compared to any other method comprising administering an equivalent dose of the therapeutic peptide. In some embodiments, the prolonged B-cell depletion comprises B cell depletion for at least 6 hours, at least 12 hours, at least 18 hours, at least 24 hours, at least 3 days, at least 4 days, at least 5 days, at least 6 days, at least 7 days, at least 8 days, at least 9 days, at least 10 days, at least 15 days, at least 17.5 days, at least 20 days, at least 25 days, at least 27.5 days, at least 30 days, at least 35 days, at least 37.5 days, at least 40 days, at least 45 days, at least 47.5 days, at least 50 days, at least 55 days, at least 57.5 days, at least 60 days, at least 65 days, at least 70 days, at least 75 days, at least 80 days, at least 85 days, at least 90 days, at least 95 days, at least 100 days, at least 105 days, at least 110 days, or at least 120 or more days.

In some embodiments, the method results in depletion of at least 50%, at least 55%, at least 60%, at least 65%, at least 70%, at least 75%, at least 80%, at least 85%, at least 90%, at least 95%, or at least 99% or more of circulating B-cells in a subject. In some embodiments, the method results in depletion of at least 50%, at least 55%, at least 60%, at least 65%, at least 70%, at least 75%, at least 80%, at least 85%, at least 90%, at least 95%, or at least 99% or more of tissue B-cells in a subject.

In some embodiments, the method results in depletion of at least 50%, at least 55%, at least 60%, at least 65%, at least 70%, at least 75%, at least 80%, at least 85%, at least 90%, at least 95%, or at least 99% or more of naive B-cells in a subject. In some embodiments, the method results in depletion of at least 50%, at least 55%, at least 60%, at least 65%, at least 70%, at least 75%, at least 80%, at least 85%, at least 90%, at least 95%, or at least 99% or more of mature B-cells in a subject. In some embodiments, the mature B-cell comprises a memory B-cell, a plasma cell, or a plasmablast.

In some embodiments, the method results in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200% or more B-cell depletion compared to any other method comprising administering an equivalent dose of the therapeutic peptide.

In some embodiments, the method further comprises administering a second pharmaceutical composition comprising an active agent. The pharmaceutical composition provided herein can be used in combination with another active agent. In some embodiments, the method further comprises administering a small molecule drug. In some embodiments, small molecule drug comprises an agent for chemotherapy.

In some embodiments, the subject in need thereof comprises a mammal. In some embodiments, the mammal comprises a human, a non-human primate, or a rodent.

In some embodiments, the method for delivering a pharmaceutical agent to a target organ in a subject in need thereof provides a greater amount or activity of the pharmaceutical agent in the target organ in the subject as compared to that achieved absent the lipid composition. In some embodiments, the method for delivering a pharmaceutical agent to a target organ in a subject in need thereof provides a greater amount or activity of the pharmaceutical agent in the target organ in the subject as compared to a non-target organ.

In some embodiments of the method, the composition of the present application can be administrated through any suitable routes comprising parenteral delivery (e.g., injections), such as intravenous, intramuscular, subcutaneous, intramedullary injections, as well as intrathecal, direct intraventricular, intraperitoneal, intranasal, or intraocular injections.

In some embodiments, the method provides potent delivery of the pharmaceutical composition to a cell of a subject. In some embodiments, the method comprising administering the pharmaceutical composition provided herein results in targeted delivery of the pharmaceutical composition to a target organ. In some embodiments, the target organ comprises liver.

In some embodiments, the method delivers a pharmaceutical composition to a target organ (e.g., liver or spleen) or a target cell (e.g., an immune cell) of a subject, and thereby providing an effective amount or activity of the pharmaceutical composition in the target organ or target cell that is at least 1.1-fold greater than a corresponding amount or activity of the pharmaceutical composition achieved in a non-target organ or non-target cell of the subject. In some embodiments, the effective amount or activity of the pharmaceutical composition in the target organ or target cell is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, at least 20-fold greater, at least 30-fold greater, at least 40-fold greater, at least 50-fold greater, at least 75-fold greater, at least 100-fold greater, at least 200-fold greater, or at least 300-fold greater, than a corresponding amount or activity of the pharmaceutical composition achieved in non-target organ or non-target cell of the subject.

In some embodiments, the methods of delivery comprise administering a pharmaceutical composition described herein provides an effective amount or activity of a pharmaceutical composition at least 1.1-fold greater than a corresponding amount or activity of the pharmaceutical composition achieved by administering other compositions. In some embodiments, the effective amount or activity of the pharmaceutical composition results from administering a lipid composition described herein is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, at least 20-fold greater, at least 30-fold greater, at least 40-fold greater, at least 50-fold greater, at least 75-fold greater, at least 100-fold greater, at least 200-fold greater, or at least 300-fold greater, than a corresponding amount or activity of the pharmaceutical composition achieved by administering other compositions.

In some embodiments, the methods of delivery comprise administering a lipid described herein provides an effective amount or activity of a pharmaceutical composition at least 1.1-fold greater than a corresponding amount or activity of the pharmaceutical composition achieved by administering other lipids. In some embodiments, the effective amount or activity of the pharmaceutical composition results from administering a lipid described herein is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, at least 20-fold greater, at least 30-fold greater, at least 40-fold greater, at least 50-fold greater, at least 75-fold greater, at least 100-fold greater, at least 200-fold greater, or at least 300-fold greater, than a corresponding amount or activity of the pharmaceutical composition achieved by administering other lipids.

In some embodiments, the delivery of the pharmaceutical composition to a cell may cause cell death, such as apoptosis of the cell.

In one aspect, provided herein is a high-potency dosage form of a pharmaceutical composition formulated with an ionizable lipid, the dosage form comprising a pharmaceutical composition (e.g., mRNA encoding a therapeutic peptide for cancer immunotherapy) assembled with a lipid composition as described herein.

The dose of the pharmaceutical composition provided herein can be measured in units of mg/kg, which refers to mg of total nucleic acid used to formulate the LNPs per kg of body weight of a subject (mg of total mRNA/kg). In some embodiments, the pharmaceutical composition is present in the dosage form at a dose of about 10, 9, 8, 7, 6, 5, 4, 3, 2, 1.5, 1.0, 0.5, 0.2, 0.1, 0.05, 0.02, 0.01, 0.005, 0.002, or 0.001 milligram per kilogram (mg/kg, or mpk) body weight, or of a range between (inclusive) any two of the foregoing values. In some embodiments, the pharmaceutical composition is present in the dosage form at a dose of no more than about 10 milligram per kilogram (mg/kg, or mpk) body weight. In some embodiments, the pharmaceutical composition is present in the dosage form at a dose of no more than about 9 mg/kg, no more than about 8 mg/kg, no more than about 7 mg/kg, no more than about 6 mg/kg, no more than about 5 mg/kg, no more than about 4 mg/kg, no more than about 3 mg/kg, no more than about 2 mg/kg, no more than about 1 mg/kg, no more than about 0.5 mg/kg, no more than about 0.2 mg/kg, no more than about 0.1 mg/kg, no more than about 0.05 mg/kg, or no more than about 0.01 mg/kg. In some embodiments, the pharmaceutical composition is present in the dosage form at a concentration of no more than about 5 milligram per milliliter (mg/mL).

In some embodiments, the pharmaceutical composition is present in the dosage form at a concentration of about 5, 4, 3, 2, 1, 0.5, 0.2, or 0.1 milligram per milliliter (mg/mL), or of a range between (inclusive) any two of the foregoing values.

In some embodiments, the pharmaceutical composition is present in the dosage form at a concentration of no more than about 5 milligram per milliliter (mg/mL). In some embodiments, the pharmaceutical composition is present in the dosage form at a concentration of no more than about 2 milligram per milliliter (mg/mL). In some embodiments, the pharmaceutical composition is present in the dosage form at a concentration of no more than about 1 milligram per milliliter (mg/mL). In some embodiments, the pharmaceutical composition is present in the dosage form at a concentration of no more than about 0.5 milligram per milliliter (mg/mL). In some embodiments, the pharmaceutical composition is present in the dosage form at a concentration of no more than about 0.1 milligram per milliliter (mg/mL).

In some embodiments, the pharmaceutical composition is present in the dosage form at a concentration of about 10, 9, 8, 7, 6, 5, 4, 3, 2, 1, 0.5, 0.2, or 0.1 microgram per milliliter (μg/mL), or of a range between (inclusive) any two of the foregoing values. In some embodiments, the pharmaceutical composition is present in the dosage form at a concentration of no more than about 10, no more than about 9, no more than about 8, no more than about 7, no more than about 6, no more than about 5, no more than about 4, no more than about 3, no more than about 2, no more than about 1, no more than about 0.5, no more than about 0.2, no more than about 0.1 microgram per milliliter (μg/mL).

Any suitable dosage form can be prepared for delivery, for example, via oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

In some embodiments, the pharmaceutical composition is administered at a dosage of no more than about 10 milligram per kilogram (mg/kg, or mpk) body weight, no more than about 9 mg/kg, no more than about 8 mg/kg, no more than about 7 mg/kg, no more than about 6 mg/kg, no more than about 5 mg/kg, no more than about 4 mg/kg, no more than about 3 mg/kg, no more than about 2 mg/kg, no more than about 1 mg/kg, no more than about 0.5 mg/kg, no more than about 0.2 mg/kg, no more than about 0.1 mg/kg, no more than about 0.05 mg/kg, or no more than about 0.01 mg/kg body weight. In some embodiments, the pharmaceutical composition is administered at a dosage from about 1 μg/kg body weight to about 3 mg/kg body weight.

In some embodiments, the administration of a dose of the lipid composition provided here can be repeated. If desired, the effective dose of the active lipid composition can be administered as one, two, three, four, five, six or more doses administered separately at appropriate intervals throughout the course of treatment. In some embodiments, the lipid composition can be administered two or three times daily. In some embodiments, the lipid composition will be administered once daily. In some embodiments, the lipid composition is administered about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, or about every 18 weeks. In some embodiments, the lipid composition is administered about every 1 month, about every 2 months, about every 3 months, about every 4 months, about every 5 months, about every 6 months, about every 7 months, about every 8 months, about every 9 months, about every 10 months, about every 11 months, about every 12 months, about every 13 months, about every 14 months, about every 15 months, about every 16 months, about every 17 months, about every 18 months, about every 2 years, about every 2.5 years, about every 3 years, about every 3.5 years, about every 4 years, about every 4.5 years, or about every 5 years. Any subject in need thereof can be treated with the method of the present application.

In some embodiments, the subject has been determined to have a mutation in a target gene. In some embodiments, the mutation in the target gene is associated with a cancer or a tumor.

In some embodiments, the subject has been determined to exhibit an aberrant expression or activity of a protein or polynucleotide that corresponds to a target gene. In some embodiments, the aberrant expression or activity of the protein or polynucleotide is associated with a cancer or a tumor.

In some embodiments, provided herein is a method for targeted delivery of a pharmaceutical agent to a cell type comprising contacting the cell with the composition of the present application. In some embodiments of the method, the pharmaceutical composition comprises a pharmaceutical agent (e.g., mRNA) assembled with a lipid composition as described in the present application, e.g., wherein the lipid composition comprises any of the head or tail groups disclosed herein.

In some embodiments, the contacting is ex vivo. In some embodiments, the contacting is in vitro. In some embodiments, the contacting is in vivo. In some embodiments, the contacting comprises administering to a subject the composition comprising the therapeutic agent assembled with the lipid composition.

In some embodiments, the method results in zero to minimum toxicity. In some embodiments, the toxicity comprises transient elevation in cytokines or liver enzymes. In some embodiments, the toxicity comprises mild inflammation or mild hepatotoxicity.

### Pharmaceutical Compositions

The compositions and methods of the present disclosure may be utilized to treat an individual in need thereof. The pharmaceutical composition described herein may comprise a therapeutic or prophylactic composition, or any combination thereof. In some embodiments, the lipid compositions can be assembled with nucleic acid sequences encoding a therapeutic peptide (e.g., BiTE). In some embodiments, the individual is a mammal (e.g., a human or a non-human mammal). Upon administration to an animal (e.g., a human or a non-human animal) the composition or the lipid composition is preferably administered as a pharmaceutical composition comprising, for example, a lipid composition of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as intravenous or intramuscular injection, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

In some embodiments, the pharmaceutical composition targets a tissue or an organ after upon administration to a subject. In some embodiments, the tissue or the organ comprises a liver. In some embodiments, the tissue or the organ comprises a lymphoid organ. In some embodiments, the lymphoid organ comprises bone marrow, spleen, or lymph nodes.

In some embodiments, the pharmaceutical composition targets a target cell upon administration to a subject. In some embodiments, the target cell comprises a hepatocyte, a lymphocyte, a leukocyte, a myeloid cell, or a hematopoietic stem cell. In some embodiments, the target cell comprises a B cell, and wherein the B cell comprises a plasmablast, a plasma cell, or a memory B cell. In some embodiments, the target cell comprises a T cell.

In some embodiments, the target cell comprises a cell in a systemic circulation of the subject. In some embodiments, the target cell comprises a cell within a tissue or an organ of the subject.

In some embodiments, the pharmaceutical composition has improved storage stability compared to other compositions.

In some embodiments, upon administration to a subject the therapeutic peptide encoded by the synthetic nucleic acid sequence of the pharmaceutical composition has a longer half-life compared to a corresponding therapeutic peptide of another pharmaceutical composition.

In some embodiments, upon administration to a subject the therapeutic peptide encoded by the synthetic nucleic acid sequence of the pharmaceutical composition has a larger area under curve (AUC) compared to an equivalent dose of a corresponding therapeutic peptide of another pharmaceutical composition.

In some embodiments, upon administration to a subject the therapeutic peptide encoded by the synthetic nucleic acid sequence of the pharmaceutical composition results in prolonged B-cell depletion compared to an equivalent dose of a corresponding therapeutic peptide of another pharmaceutical composition.

In some embodiments, upon administration to a subject the therapeutic peptide encoded by the synthetic nucleic acid sequence of the pharmaceutical composition results in depletion of at least 50%, at least 55%, at least 60%, at least 65%, at least 70%, at least 75%, at least 80%, at least 85%, at least 90%, at least 95%, or at least 99% or more of circulating B-cells in a subject. In some embodiments, after being administered to a subject the therapeutic peptide encoded by the synthetic nucleic acid sequence of the pharmaceutical composition results in depletion of at least 50%, at least 55%, at least 60%, at least 65%, at least 70%, at least 75%, at least 80%, at least 85%, at least 90%, at least 95%, or at least 99% or more of tissue B-cells in a subject.

In some embodiments, upon administration to a subject the therapeutic peptide encoded by the synthetic nucleic acid sequence of the pharmaceutical composition results in depletion of at least 50%, at least 55%, at least 60%, at least 65%, at least 70%, at least 75%, at least 80%, at least 85%, at least 90%, at least 95%, or at least 99% or more of naive B-cells in a subject. In some embodiments, after being administered to a subject the therapeutic peptide encoded by the synthetic nucleic acid sequence of the pharmaceutical composition results in depletion of at least 50%, at least 55%, at least 60%, at least 65%, at least 70%, at least 75%, at least 80%, at least 85%, at least 90%, at least 95%, or at least 99% or more of mature B-cells in a subject.

In some embodiments, upon administration to a subject the therapeutic peptide encoded by the synthetic nucleic acid sequence of the pharmaceutical composition results in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200% or more B-cell depletion compared to an equivalent dose of a corresponding therapeutic peptide of another pharmaceutical composition.

In some embodiments, upon administration to the subject the pharmaceutical composition has zero to minimum toxicity. In some embodiments, the toxicity comprises transient elevation in cytokines or liver enzymes. In some embodiments, the toxicity comprises mild inflammation or mild hepatotoxicity.

In some embodiments, upon administration to a subject the pharmaceutical composition has a faster rate of clearance compared to other compositions.

In some embodiments, the pharmaceutical composition further comprises a pharmaceutically acceptable excipient. In some embodiments, the pharmaceutically acceptable excipient comprises a solution suitable for injections into a subject. In some embodiments, the pharmaceutical composition further comprises a small molecule drug. In some embodiments, the small molecule drug comprises an agent for chemotherapy.

The pharmaceutical composition provided herein can comprise 1) the anti-CD19 light chain coding region comprising a nucleic acid sequence having 90% sequence identity to SEQ ID NO: 1, and the anti-CD19 heavy chain coding region comprising a nucleic acid sequence having 90% sequence identity to SEQ ID NO: 2; and 2) the anti-CD3 light chain coding region comprising a nucleic acid sequence having 90% sequence identity to SEQ ID NO: 3, and the anti-CD3 heavy chain coding region comprising a nucleic acid sequence having 90% sequence identity to SEQ ID NO: 4.

In some embodiments, upon administration to the subject, the pharmaceutical composition results in activation of CD69+ T cells.

In some embodiments, the physical properties of the pharmaceutical composition are stable for at least 2 months, at least 3 months, at least 4 months, at least 5 months, or at least 6 months or more when stored at 4° C., −20° C., or −80° C. In some embodiments, the physical properties of the pharmaceutical composition are stable for at least 5 months when stored at 4° C., −20° C., or −80° C. In some embodiments, the physical properties of the pharmaceutical composition comprise size, polydispersity index (PDI), encapsulation efficacy (EE %), or pKa of the lipid composition.

A pharmaceutically acceptable carrier can contain physiologically acceptable agents that act, for example, to stabilize, increase solubility or to increase the absorption of a lipid composition such as a lipid composition of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a lipid composition of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those lipid compositions, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, mannose, trehalose, and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The lipid composition may also be formulated for inhalation. In some embodiments, a lipid composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the lipid composition which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition, such as a lipid (e.g., nanoparticle) composition as described herein, with the carrier and, optionally, one or more accessory ingredients. In general, the formulations are prepared by uniformly and intimately bringing into association a lipid (e.g., nanoparticle) composition as described herein with liquid carriers, or finely divided solid carriers, or both, and then, if necessary, shaping the product.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. In some embodiments, the pharmaceutical compositions comprising the mRNA/LNPs provided herein are administered through parenteral routes (e.g., intravenous injection or intramuscular injection). Pharmaceutical compositions suitable for parenteral administration comprise one or more active lipid compositions in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject lipid compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active lipid compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a lipid composition at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular lipid composition or combination of lipid compositions employed, or the ester, salt or amide thereof, the route of administration, the time of administration, the rate of excretion of the particular lipid composition(s) being employed, the duration of the treatment, other drugs, lipid compositions and/or materials used in combination with the particular lipid composition(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or lipid composition at levels lower than that required in order to achieve the desired therapeutic effect and gradually increase the dosage until the desired effect is achieved. By “therapeutically effective amount” is meant the concentration of a lipid composition that is sufficient to elicit the desired therapeutic effect. It is generally understood that the effective amount of the lipid composition will vary according to the weight, sex, age, and medical history of the subject. Other factors which influence the effective amount may include, but are not limited to, the severity of the patient's condition, the disorder being treated, the stability of the lipid composition, and, if desired, another type of therapeutic agent being administered with the lipid composition of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active lipid composition used in the compositions and methods of the invention will be that amount of the lipid composition that is the lowest dose effective to produce a therapeutic effect. Such an effective dose will generally depend upon the factors described above.

If desired, the effective dose of the active lipid composition may be administered as one, two, three, four, five, six or more doses administered separately at appropriate intervals throughout the course of treatment, optionally, in unit dosage forms. In some embodiments of the present invention, the active lipid composition may be administered two or three times daily. In some embodiments, the active lipid composition will be administered once daily.

The patient or subject receiving this treatment is any animal in need, including primates, in particular humans; and other mammals such as equines, cattle, swine, sheep, cats, and dogs; poultry; and pets in general.

In some embodiments, lipid compositions of the invention may be used alone or conjointly administered with another type of therapeutic agent.

The present disclosure includes the use of pharmaceutically acceptable salts of lipid compositions of the invention in the compositions and methods of the present invention. In some embodiments, contemplated salts of the invention include, but are not limited to, alkyl, dialkyl, trialkyl or tetra-alkyl ammonium salts. In some embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In some embodiments, contemplated salts of the invention include, but are not limited to, Na, Ca, K, Mg, Zn or other metal salts. In some embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d glucoheptonic acid, d gluconic acid, d glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1 tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Kits

Another aspect provides a kit comprising the pharmaceutical composition and/or lipid nanoparticle (LNP) formulations described herein. In some embodiments, the kit can further comprise an apparatus for administering the compositions provided herein, such as appropriate syringes for intravenous or intramuscular administration.

In any herein-disclosed method, the method further comprises providing instructions for use (IFU), the IFU including instructions for administering the LNP compositions to a subject. In some embodiments, the user instruction directs a user to inject the LNP compositions comprising mRNA intravenously or intramuscularly for use in immunotherapy.

### Definitions

Before the embodiments of the disclosure are described, it is to be understood that such embodiments are provided by way of example only, and that various alternatives to the embodiments of the disclosure described herein may be employed in practicing the disclosure. Numerous variations, changes, and substitutions will now occur to those skilled in the art without departing from the disclosure.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the disclosure.

In the context of the present application, the following terms have the meanings ascribed to them unless specified otherwise:

As used throughout the specification and claims, the terms “a”, “an” and “the” are generally used in the sense that they mean “at least one”, “at least a first”, “one or more” or “a plurality” of the referenced components or steps, except in instances wherein an upper limit is thereafter specifically stated. For example, a “cleavage sequence”, as used herein, means “at least a first cleavage sequence” but includes a plurality of cleavage sequences. The operable limits and parameters of combinations, as with the amounts of any single agent, will be known to those of ordinary skill in the art in light of the present application.

The terms “polypeptide”, “peptide”, and “protein” are used interchangeably herein to generally refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein, the term “antibody” refers to an immunoglobulin (Ig) whether natural or partly or wholly synthetically produced. The term also covers any polypeptide or protein having a binding domain which is, or is homologous to, an antigen-binding domain. The term further includes “antigen-binding fragments” or “functional fragment thereof”, or “fragment of an antibody”, “antibody fragment”, “functional fragment of an antibody” and other interchangeable terms for similar binding fragments such as described below. An antibody includes, for example, monoclonal antibodies, chimeric antibodies, humanized antibodies, human antibodies, recombinant antibodies, chemically engineered antibodies, deimmunized antibodies, affinity-matured antibodies, multispecific antibodies (for example, bispecific antibodies and polyreactive antibodies), heteroconjugate antibodies, antibody fragments, and combinations thereof (e.g., a monoclonal antibody that is also deimmunized, a humanized antibody that is also deimmunized, etc.). An antibody can be, for example, murine, chimeric, humanized, heteroconjugate, bispecific, diabody, triabody, or tetrabody. The antigen-binding fragment can include, for example, Fab′, F(ab′)2, Fab, Fv, rlgG, scFv, hcAbs (heavy chain antibodies), a single domain antibody, VHH, VNAR, sdAbs, or nanobody.

The term “antigen” refers to a molecule bound by an antibody or a fragment thereof. In some cases, the antigen can be referred to as a “ligand” of the antibody. An antigen can be derived from a surface protein of a cell, such as an immune cell or a cancer cell. In some cases, an antigen can be derived from a surface protein of a tumor cell. An antigen can be targeted by the antibody for killing of a cell.

As used herein, the terms “treatment” or “treating,” or “palliating” or “ameliorating” are used interchangeably herein. These terms generally refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms or improvement in one or more clinical parameters associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect” or “therapeutic benefit,” as used herein, generally refers to a physiologic effect, including but not limited to the mitigation, amelioration, or prevention of disease or an improvement in one or more clinical parameters associated with the underlying disorder in humans or other animals, or to otherwise enhance physical or mental wellbeing of humans or animals, resulting from administration of a polypeptide of the disclosure other than the ability to induce the production of an antibody against an antigenic epitope possessed by the biologically active protein. For prophylactic benefit, the compositions may be administered to a subject at risk of developing a particular disease, a recurrence of a former disease, condition or symptom of the disease, or to a subject reporting one or more of the physiological symptoms of a disease, even though a diagnosis of this disease may not have been made.

The terms “therapeutically effective amount” and “therapeutically effective dose”, as used herein, generally refer to an amount of a drug or a biologically active protein, either alone or as a part of a polypeptide composition, that is capable of having any detectable, beneficial effect on any symptom, aspect, measured parameter or characteristics of a disease state or condition when administered in one or repeated doses to a subject. Such effect need not be absolute to be beneficial. Determination of a therapeutically effective amount is well within the capability of those skilled in the art, especially in light of the detailed disclosure provided herein.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl(C≤8)” or the class “alkene(C≤8)” is two. Compare with “alkoxy(C≤10)”, which designates alkoxy groups having from 1 to 10 carbon atoms. “Cm-n” or “Cm-Cn” defines both the minimum (m) and maximum number (n) of carbon atoms in the group. Thus, “C1-C10 alkyl” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” generally signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the class of compounds having the formula H-R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂C1, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C1. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, the carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no

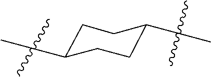 [SMILES: *C1CCC(*)CC1]

atoms other than carbon and hydrogen. The group is a non-limiting example of cycloalkanediyl group. A “cycloalkane” refers to the class of compounds having the formula H-R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H-R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H-R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, the carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, the carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

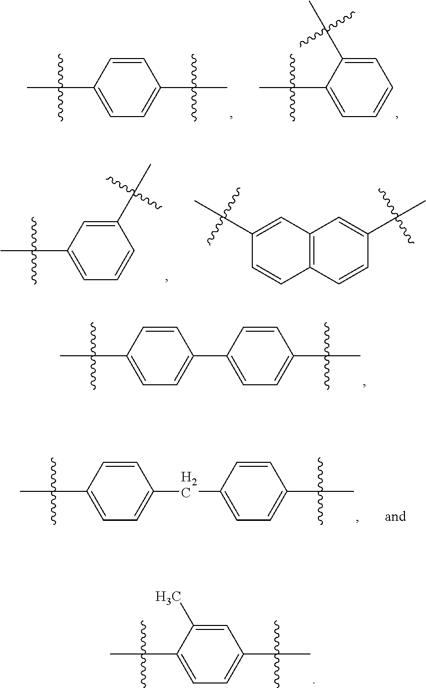

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “hetero” when used to modify a compound or chemical group means the compound or chemical group has at least an atom that is not carbon, for example, N, O, S, Se, P, Si, B, or any other heteroatom. For example, a heteroaliphatic can be any aliphatic moiety containing at least one heteroatom selected from N, O, P, B, S, Si, Sb, Al, Sn, As, Se, and Ge. A heterocycle can be any ring containing a ring atom that is not carbon. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

The term “heteroaryl” when used without the “substituted” modifier refers to a monovalent aromatic group with an aromatic carbon atom or nitrogen atom as the point of attachment, the carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, the atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

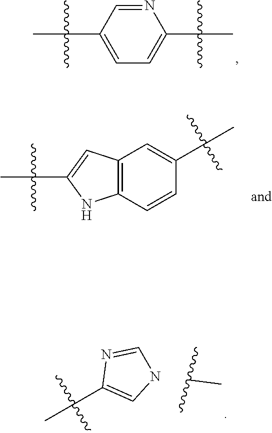

The term “heterocycloalkyl” when used without the “substituted” modifier refers to a monovalent non-aromatic group with a carbon atom or nitrogen atom as the point of attachment, the carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to a divalent cyclic group, with two carbon atoms, two nitrogen atoms, or one carbon atom and one nitrogen atom as the two points of attachment, the atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

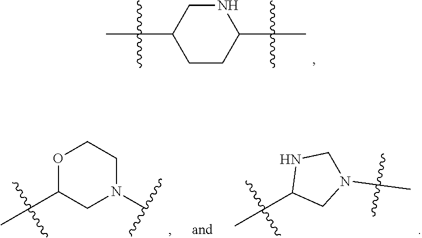 [SMILES: *C1CCC(*)NC1 ; *C1CN(*)CCO1 ; *C1CN(*)CN1]

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃.

The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. Unless specified otherwise, aliphatic, heteroaliphatic, oxyaliphatic, alkyl, alkylene, alkenyl, alkenylene, alkynyl, alkynylene, cycloalkyl, cycloalkylene, cycloalkenyl, cycloalkenylene, cycloalkynyl, cycloalkynylene, hydroxyalkyl, heterocycloalkyl, heterocycloalkylene, heterocycloalkenyl, heterocycloalkenylene, aryl, and heteroaryl mentioned herein include both substituted and unsubstituted moieties.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally, the term “about,” as used herein when referring to a measurable value such as an amount of weight, time, dose, etc. is meant to encompass in one example variations of ±20% or ±10%, in another example ±5%, in another example ±3%, in another example ±1%, and in yet another example ±0.1% from the specified amount, as such variations are appropriate to perform the disclosed method.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate (e.g., non-human primate). In certain embodiments, the patient or subject is a human. Non-limiting examples of human subjects are adults, juveniles, infants and fetuses.

The term “assemble” or “assembled,” as used herein, in context of delivery of a payload to target cell(s) generally refers to covalent or non-covalent interaction(s) or association(s), for example, such that a therapeutic or prophylactic agent be complexed with or encapsulated in a lipid composition.

As used herein, the term “lipid composition” generally refers to a composition comprising lipid compound(s), including but not limited to, a lipoplex, a liposome, a lipid particle. Examples of lipid compositions include suspensions, emulsions, and vesicular compositions.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present application which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically acceptable material, composition or vehicle, such as a liquid or solid filler, diluent, excipient, solvent or encapsulating material, involved in carrying or transporting a chemical agent.

The term “helper lipid” as used in this disclosure refers to a lipid that contributes to the stability or delivery efficacy of a lipid composition. A helper lipid can be a zwitterionic lipid, such as a phospholipid. A helper lipid can be phosphatidylcholine, distearoylphosphatidylcholine, dioleoylphosphatidylethanolamine, 1,2-dioleoyl-sn-glycero-3-phosphoethanolamine (DOPE) or 1,2-dioleoyl-sn-glycero-3-phosphocholine (DOPC). In some cases, the term “helper lipid” refers to phospholipids or other zwitterionic lipids in the LNP composition. In some cases, when describing the formulation of an LNP using weight ratios of the lipid components (e.g., lipidoid, steroid, helper lipid, and polymer conjugated lipid), a helper lipid refers to a phospholipid or another zwitterionic lipid. For example, the weight ratio of the lipidoid/steroid/helper lipid/polymer conjugated lipid is about 4/1/1/1. “Helper lipid” can refer to any class of lipid molecules that improves the particle stability and fluidity of lipid nanoparticles (LNP). Several classes of molecules can be used as helper lipids such as phospholipids (e.g., phosphoethanolamine, phosphocholine), zwitterionic lipids, steroid derivatives, and polymer conjugated lipids (e.g., PEGylated lipid). Representative helper lipids include cholesterol, 1,2-dioleoyl-sn-glycero-3-phosphoethanolamine (DOPE), 1,2-dioleoyl-sn-glycero-3-phosphocholine (DOPC), Phosphatidylcholine (PC), Methoxy-Polyethyleneglycol (MW 2k)-distearoylphosphatidylethanolamine (mPEG2k-DSPE), and 1,2-dimyristoyl-rac-glycero-3-methoxypolyethylene glycol-2000 (DMG-PEG2k).

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Lipid Nanoparticle (LNP) Formulation and Characterization

Table 4 lists some of the lipids tested. All test lipids were synthesized through a solvent free Michael Addition reaction between an amine head and an alkyl-acrylate lipid tail.

In the cases of homogenous tailed lipids, which comprises one kind of hydrophobic tails, the aliphatic amine head (3,3′-Diamino-N-methyldipropylamine) and the corresponding acrylate tail were mixed at 1 to 5 molar ratio in Teflon-lined glass screw-top vials at 70° C. for 48 h. The crude products were purified using a Teledyne Isco Chromatography system using the mobile phase of methanol/DCM. The purified lipidiods were characterized by electrospray ionization mass spectrometry (ESI-MS).

In the cases of asymmetric heterogenous lipids, the amine head was mixed with the major lipid tail at 1 to 3.5 molar ratio. After 48 hours stirring at 70° C., three tailed lipids were purified similarly and reacted with the fourth tail subsequently at 1: 1.5 molar ratio. The final products were isolated and verified by ESI-MS.

In the cases of symmetric heterogenous lipids, boc protected amine head was reacted with one lipid tail at 1 to 2.5 molar ratio. After mixing at 70° C. for 48 hours, the half assembled lipid was purified, boc-protection group was removed by trifluoroacetic acid in DCM, and the half lipid was further reacted with the second lipid tail to afford the full heterogenous but symmetric lipid product.

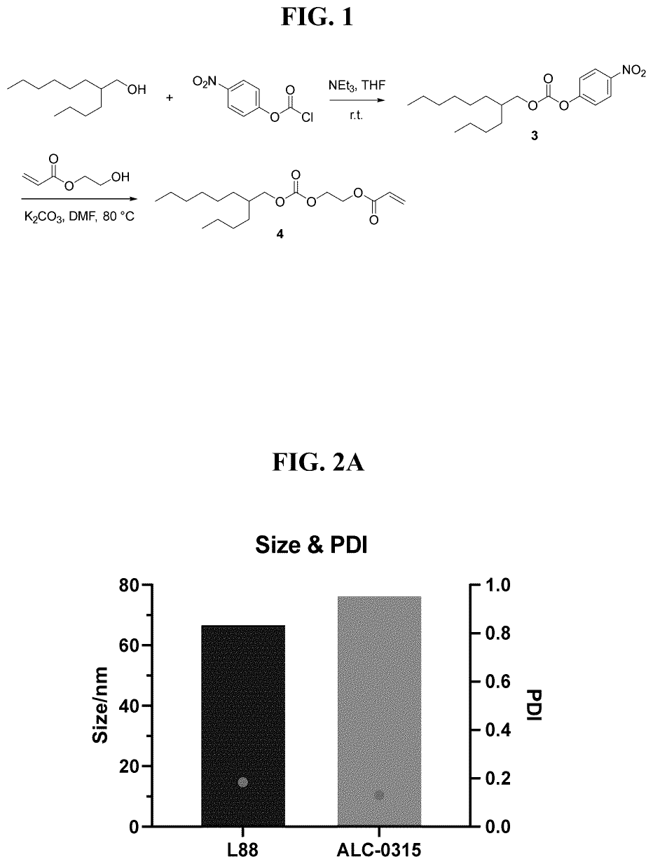

FIG. 1 illustrates the synthesis of lipid L0088 tail. 4-Nitrophenyl chloroformate was added to a solution of 2-butyl-1-octanol and triethylamine in THF (500 ml) under Argon in a round flask with a magnetic stir bar at rt. The reaction mixture was stirred for 2-3 hours until completion, monitored by TLC. Then the reaction mixture was diluted with ethyl acetate, and washed successively with 1 N HCl solution, saturated NaHCO₃ solution and brine. The organic layers were dried over MgSO₄, filtered and concentrated. The residue was purified by flash column chromatography on silica (0-10% ethyl acetate in hexanes) to afford 3 (88% yield).

Reagent 3 was added to a solution of 2-hydroxyethyl acrylate in DMF under Argon. The reaction mixture was heated to 50° C., and K₂CO₃ was added to the reaction mixture after 10 min. Then the whole reaction mixture was heated at 80° C. for 2-3 hours until completion, checked by TLC. DMF was removed by rotary evaporation and the residue was partitioned by addition of water and ethyl acetate. The aqueous layer was extracted with ethyl acetate three times. The combined organic layers were washed with water and brine, dried over Na₂SO₄, filtered and concentrated. The residue was purified carefully by flash column chromatography on silica (10-30% ethyl acetate in hexanes) to afford 4 (70%).

### Example 2. Formulation of BiTE Encoding mRNA Using Cationic Lipid Nanoparticles

Test lipids in TABLE 4 were formulated with cholesterol, DOPC, DMG-PEG, and mRNA encoding CD19-CD3 BiTE. In general, component lipids (ionizable cationic lipids, cholesterol, DOPC, and DMG-PEG) were dissolved in ethanol at appropriate molar ratios of 46/42/10/1.6. Each lipid was dissolved in ethanol and mixed to reach the specified molar ratios in the organic phase. Nucleic acids were dissolved in 25 mM sodium acetate pH 5.2 buffer to reach 0.134 mg/mL. The aqueous and organic solutions were mixed in the NanoAssemblr® (Precision NanoSystems) at a flow rate ratio of 3:1 (v/v; respectively) and a total flow rate of 20 mL/min. The resultant mixture was dialyzed directly against at least 1000-fold volume of 10 mM Tris-HCl pH 7.5 buffer overnight using a Slide-A-Lyzer MINI Dialysis Device (MWCO, 3.5 KDa). Sucrose was then added to formulations to the final concentration of 1.32 mg/mL followed by 0.22 μm filtration and stored in −4° C., −80° C. for short-term and long-term storage, respectively.

TABLE 4
Non-limiting exemplary chemical structures of ionizable lipids
tested

| Entry | Chemical | |
|---|---|---|
| # | Name | Structure |
| 1 | L0088 | 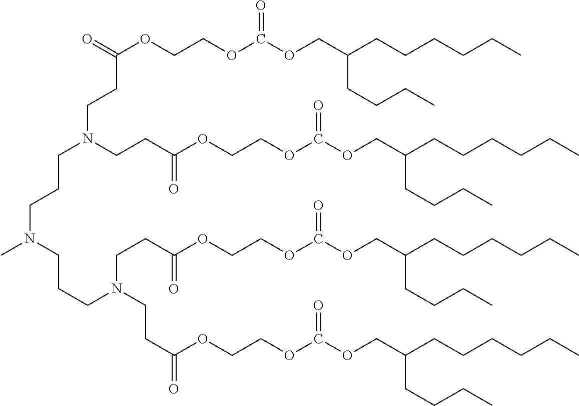 [SMILES: CCCCCCC(CCCC)COC(=O)OCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] |
| 2 | L0093 | 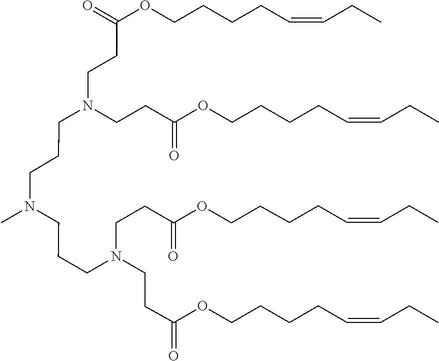 [SMILES: CC/C=C\CCCCOC(=O)CCN(CCCN(C)CCCN(CCC(=O)OCCCC/C=C\CC)CCC(=O)OCCCC/C=C\CC)CCC(=O)OCCCC/C=C\CC] |
| 3 | L0010 | 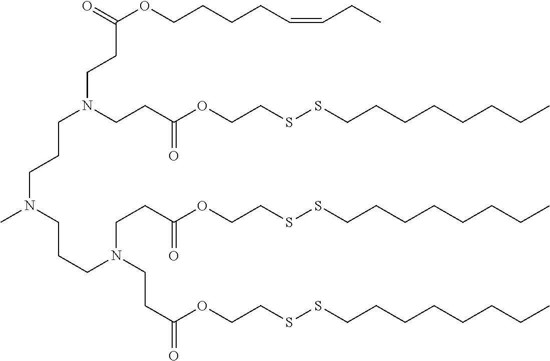 [SMILES: CC/C=C\CCCCOC(=O)CCN(CCCN(C)CCCN(CCC(=O)OCCSSCCCCCCCC)CCC(=O)OCCSSCCCCCCCC)CCC(=O)OCCSSCCCCCCCC] |
| 4 | L0065 | 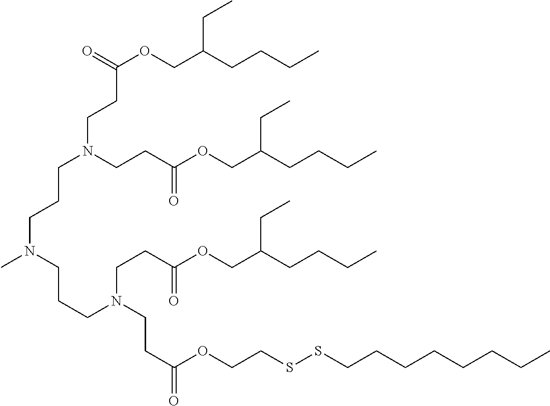 [SMILES: CCCCCCCCSSCCOC(=O)CCN(CCCN(C)CCCN(CCC(=O)OCC(CC)CCCC)CCC(=O)OCC(CC)CCCC)CCC(=O)OCC(CC)CCCC] |
| 5 | L0090 | 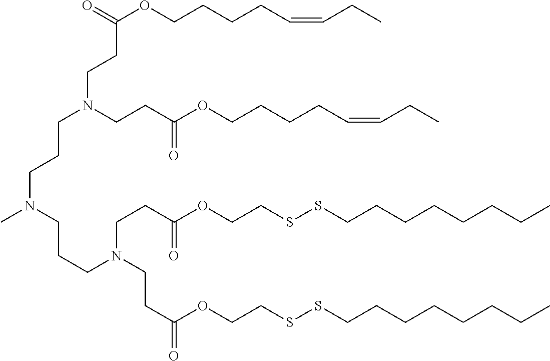 [SMILES: CC/C=C\CCCCOC(=O)CCN(CCCN(C)CCCN(CCC(=O)OCCSSCCCCCCCC)CCC(=O)OCCSSCCCCCCCC)CCC(=O)OCCCC/C=C\CC] |
| 6 | L0095 | 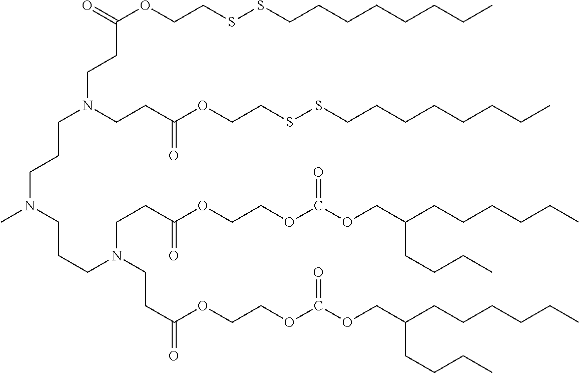 [SMILES: CCCCCCCCSSCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCSSCCCCCCCC] |
| 7 | L0105 | 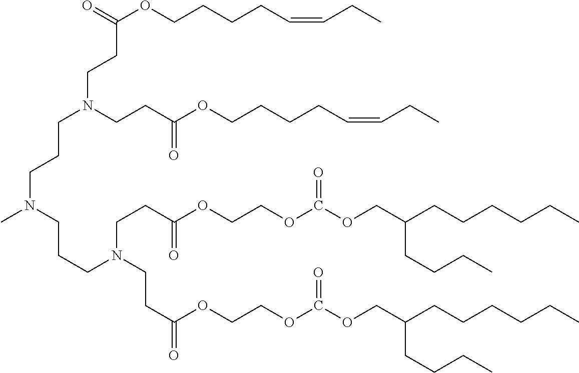 [SMILES: CC/C=C\CCCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCCC/C=C\CC] |
| 8 | L0465 | 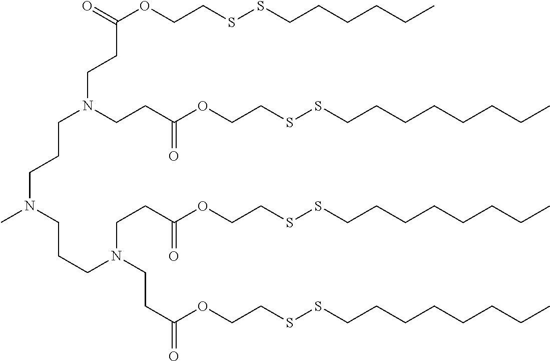 [SMILES: CCCCCCCCSSCCOC(=O)CCN(CCCN(C)CCCN(CCC(=O)OCCSSCCCCCCCC)CCC(=O)OCCSSCCCCCCCC)CCC(=O)OCCSSCCCCCC] |
| 9 | Pfizer ALC- 0315 | 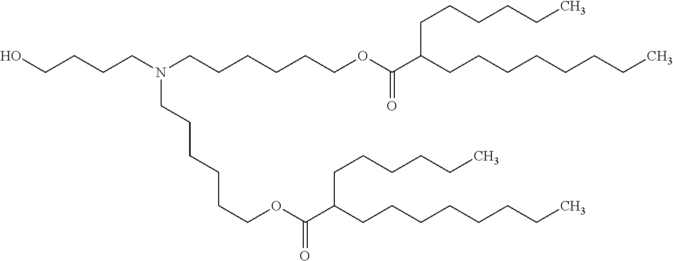 [SMILES: CCCCCCCCC(CCCCCC)C(=O)OCCCCCCN(CCCCO)CCCCCCOC(=O)C(CCCCCC)CCCCCCCC] |
| 10 | Pfizer ALC- 0159 | 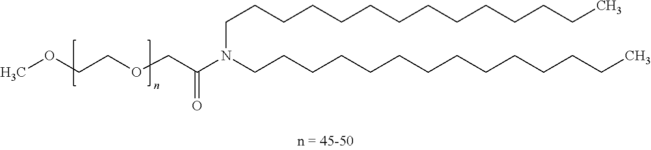 |

### Example 3. Characterization of Cationic Lipid Nanoparticles

LNPs were formulated with different cationic lipids (test lipids and a reference lipid) and mRNA encoding luciferase, and the physical properties of the LNPs were evaluated. The particle size, polydispersity index (PDI), and encapsulation of all lipid nanoparticles were characterized. LNPs were diluted in PBS pH=7.4 and transferred into a quartz cuvette (ZEN2112) to measure size by Dynamic Light Scattering (DLS) in the Zetasizer Nano ZS (Malvern Panalytical) using particle RI of 1.45 and absorption of 0.001 in PBS at 25° C. with viscosity of 0.888 cP and RI of 1.335. Measurements were made using a 173′ backscatter angle of detection previously equilibrated to 25° C. for 30 s in duplicates, each with 5 runs and 10 s run duration, without delay between measurements. Each measurement had a fixed position of 4.65 mm in the quartz cuvette with an automatic attenuation selection. Encapsulation efficiency (EE %) was done by using commercial Invitrogen™ Quant-it™ RiboGreen RNA Assay Kit. LNP formulations were diluted 250 times using TE buffer or Triton/TE (0.5% v/v Triton in TE buffer) and incubated at room temperature for 30 min to extract LNPs with Triton before adding to the 96 well plate. Microplates were then immediately introduced into the SpectraMax® iD3 plate reader to read fluorescence at Ex485/Em530). % of encapsulation efficiency were calculated as [Total mRNA−free mRNA]/[Total mRNA]×100%. The surface pKa values of LNPs were determined based on TNS assay. Briefly, solutions of 20 mM sodium phosphate, 25 mM citrate, 20 mM ammonium acetate and 150 mM NaCl were titrated to pH values varying by 0.5 from 2.0 to 12.0 and aliquoted into a black 96-well plate. LNPs and 2-(p-toluidinyl) naphthalene-6-sulfonic acid (TNS, Sigma Aldrich) were diluted into these solutions for a final concentration of 20 uM and 6 uM, respectively. Fluorescence intensity was read on a plate reader at an excitation of 322 nm and an emission of 431 nm. pKa values were calculated as the pH corresponding to 50% LNP protonation, assuming minimum and maximum fluorescence values corresponding to zero and 100% protonation, respectively.

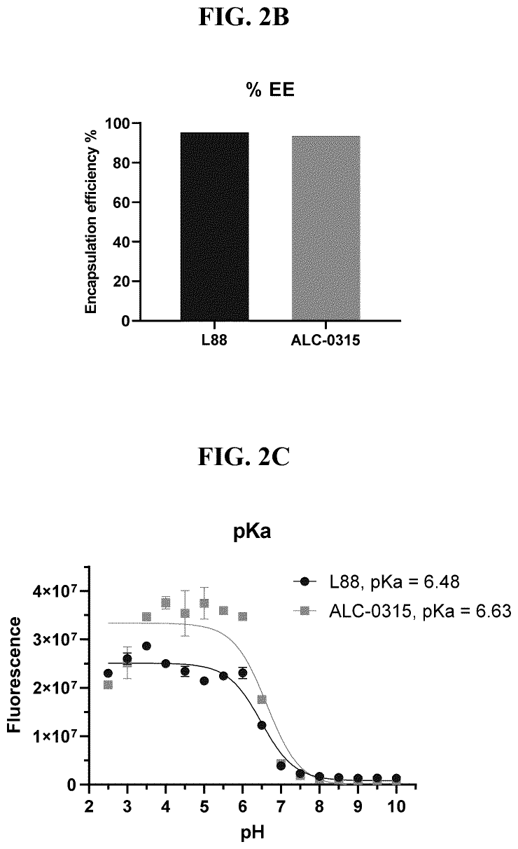

As shown in FIG. 2A, the particle sizes and PDI of test LNPs and reference LNPs were comparable and not significantly different. As shown in FIG. 2B, encapsulation efficiency (EE %) of test and reference LNPs were comparable and not significantly different. As shown in FIG. 2C, pKa of test LNPs and reference LNP were comparable and not significantly different. Overall, the test LNPs and reference LNP were comparable in physical properties.

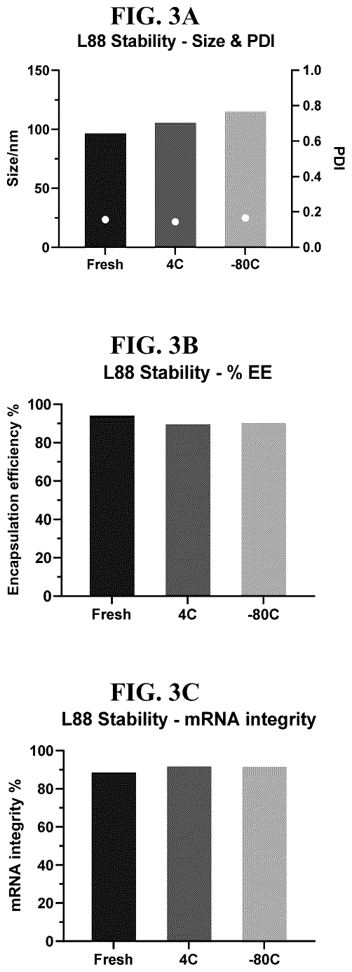

Size, PDI, pKa, EE % of the LNPs were measured over time to evaluate the stability of the LNPs at different storage conditions. The integrity of mRNA was analyzed by a fragment analysis assay over time. We observed that when stored at 4° C. or −80° C., the physical and chemical properties of LNPs formulated with the ionizable lipids are stable over time. As depicted in FIG. 3A, FIG. 3B, and FIG. 3C, LNPs comprising L0088 and mRNA encoding CD19/CD3 BiTE (e.g., L88-CD19/CD3 BiTE) maintained stable sizes, PDI, pKa, EE % and mRNA integrity for up to 5 months.

### Example 4: Characterization of LNP in Mice

### Tissue and Cellular Tropism

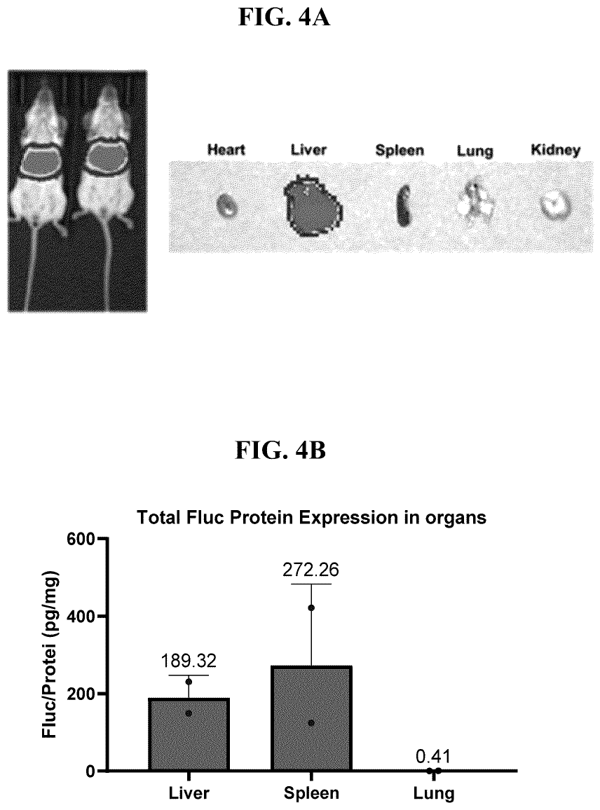

All animal experiments were conducted in accordance with the approved animal protocols. LNPs were formulated with lipids and mRNA encoding Luciferase (Luc LNPs) were injected intravenously (i.v.) to CD1 mice at 0.5 mg/kg body weight. Six hours post injection, 100 μL of luciferin K salt (15 mg/mL in PBS) was administered to the mice through intraperitoneal injections. The mice were then imaged using the In Vivo Imaging System (IVIS) and the total flux of areas of interest was quantified. Multiple mouse organs were harvested at 6 hours after i.v. injection of the Luc mRNA/LNPs. Tissues were homogenized and proteins were extracted. Levels of luciferase was measured by a luciferase assay. As depicted in FIG. 4A, i.v. injections of L88 LNP comprising Luc mRNA (e.g., L88-Luc LNP) to the mice resulted in in vivo production of Luciferase, primarily observed in the liver and spleen. Distribution to the liver and the spleen was also observed in tissue lysates, as depicted in FIG. 4B.

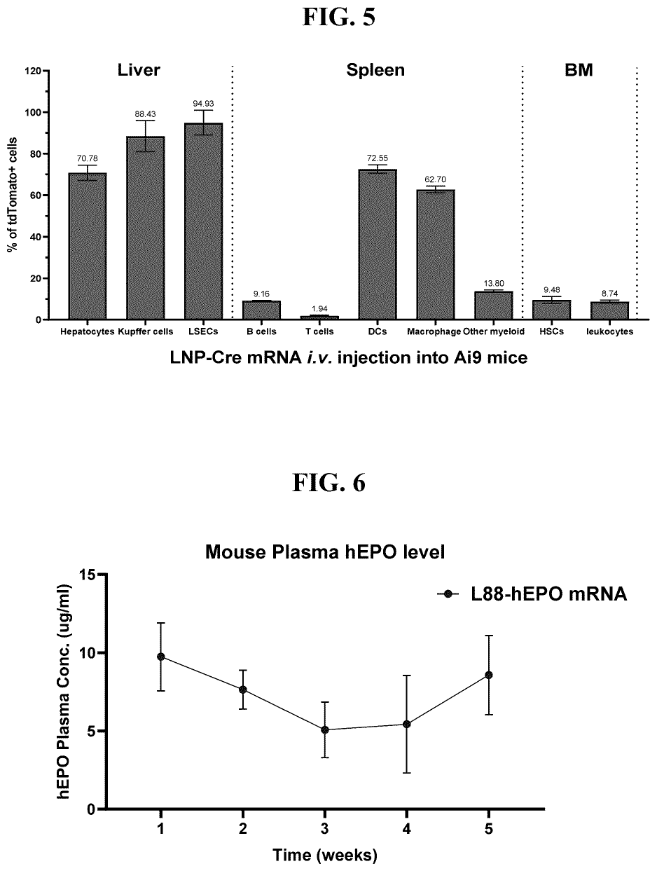

L88 LNP comprising Cre mRNA (L88-Cre LNP) was injected intravenously at Ai9 mice at 0.5 mg/kg. after 1 week, mouse organs were harvested and bone marrows were also extracted. Tissues were treated with enzymes such as papain to obtain single cell suspension. Isolated cells were stained using antibody mixture and analyzed by flow cytometry to identify distribution of the L88-Cre LNP. FIG. 5 shows the cellular tropism of the L88 LNP. L88 LNP specifically targeted some cells but not others.

Protein Expression Over Time after Repeated Dose of L88 LNP.

L88 LNP comprising mRNA encoding human erythropoietin (hEPO) (e.g., L88-hEPO LNP) was injected intravenously to CD1 mice at 0.5 mg/kg dose. A dose was administered to the same mice once a week for at least 5 weeks. Plasma hEPO protein levels were quantified by ELISA at the same time after each injection. FIG. 6 shows the changes in plasma hEPO over time.

### Pharmacokinetics of LNPs.

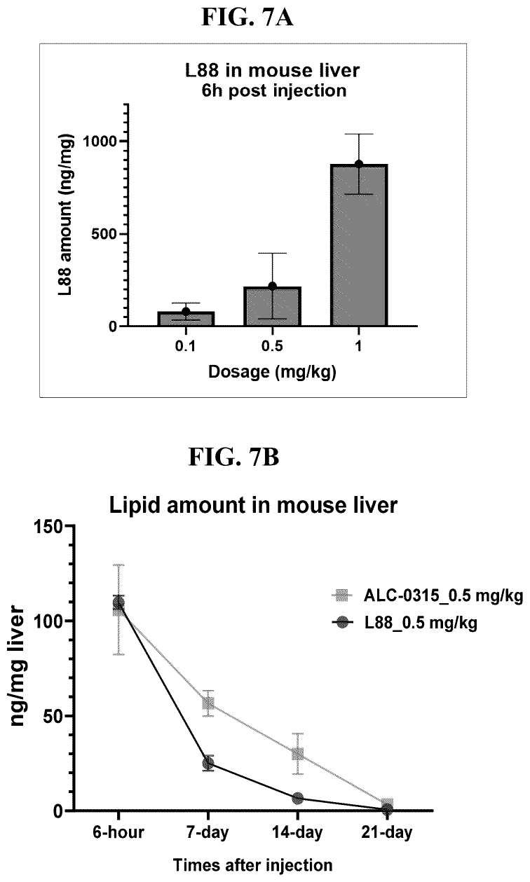

Luc LNPs were injected intravenously to CD1 mice at 0.5 mg/kg body weight. Liver tissue was obtained at 6-hour, on day 7, on day 14, and on day 21 post injection. Lipids were extracted for analysis via liquid chromatography-mass spectrometry (LC-MS). The amount of lipids (ng of lipids per kg of liver) was plotted over time, as depicted in FIG. 7A and FIG. 7B. L88-LNP had a faster clearance than the reference LNP.

### Example 5. L88-CD19/CD3 BiTE: Construct Design and In Vivo Expression and Testing

Human codon-optimized DNA sequences were generated by gene synthesis. VH and VL sequences derived from different anti-CD19 and anti-CD3 clones were fused via a 3× glycine-serine (GS) linker. The scFv sequences of anti-CD19 and anti-CD3 were linked by a 5-residue GS linked. All Bispecific T cell engager (BiTE) constructs contained a secretion signal and C-terminal 6×His tag. RNA sequences are listed in TABLE 1. Multiple mRNA constructs were synthesized comprising the anti-CD19 encoding region of SEQ ID NOs 1-2, SEQ ID NOs 5-6, or SEQ ID NOs 9-10. The mRNA constructs comprise the anti-CD3 encoding regions of SEQ ID NOs: 3-4 or SEQ ID NOs: 7-8.

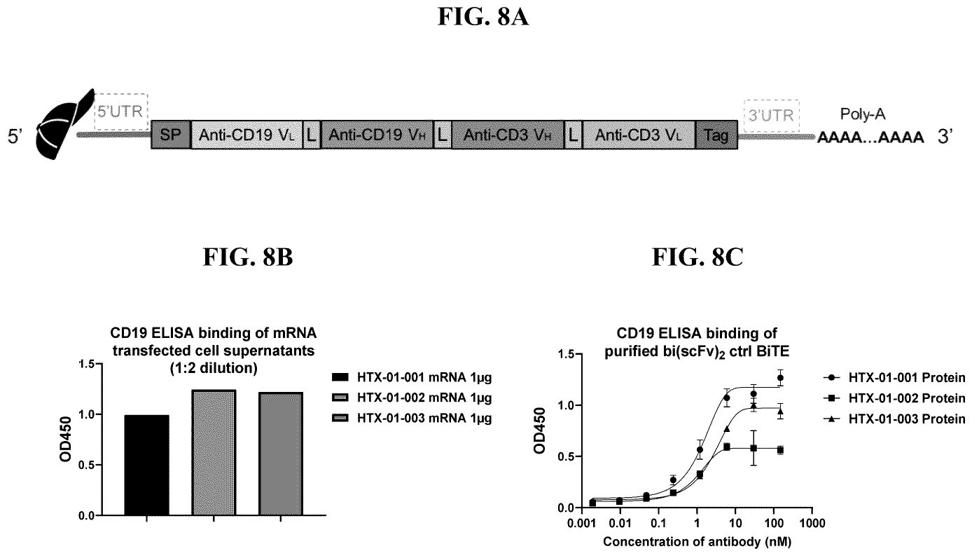

BiTE encoding mRNAs were synthesized at TriLink Biotechnology. FIG. 8A represents the structure for BiTE encoding mRNA. All mRNAs have a 5′-cap (Cap1), a 3′ poly A tail and are modified with pseudouridine. 5′ and 3′-UTRs flank the CD19-CD3 BiTE mRNA coding sequence in each mRNA.

In vitro expression of mRNA-encoded CD19-CD3 BiTE was analyzed by lipofection of the mRNA into HEK293 cells and subsequently determined the by western blot. In general, one day prior to lipofection, HEK293 cells were seeded in 24-well TC culture plate with 1 ml DMEM+10% FBS. For lipofection, mRNA and lipofectamine mixture in 1:2 ratio was prepared under sterile, RNase-free conditions and applied to the HEK293 cells at approximately 60-70% confluency. After 48 h incubation at 37° C. and 5% CO2, supernatants were collected and stored at −20° C. until further use. Cell supernatants containing secreted CD19-CD3 BiTEs were measured via western blot using a horseradish peroxidase (HRP)-conjugated antibody against 6×His.

To assess binding capacity of mRNA-encoded CD19-CD3 BiTE towards the human CD19, CD3 protein as well as dual binding of two arms, ELISA assays were performed using supernatants from mRNA transfected HEK293 cells as well as the corresponding purified recombinant CD19-CD3 BiTE protein. 96 well of ELISA plates were coated with indicated capture antigen protein overnight at 4° C. After brief washing and blocking, 100 μl of proper diluted samples were add into the plate for 2 hours incubation at room temperature. After aspirating and washing 3 times, 100 μl prepared detector antibody anti-6×His-HRP mixtures were added into each well for 1-hour incubation at room temperature. After aspirating and washing/soaking 3 times, TMB substrate solution was added, and the HRP reaction was stopped using 2N H2SO4. Absorbance was read at 450 nm by a SpectraMax iD3 plate reader. The binding curve of purified recombinant CD19-CD3 BiTE protein and secreted protein in supernatants using corresponding protein as concentration standard were analyzed by Prism software.

CD19 ELISA binding results were shown in FIG. 8B and FIG. 8C. In conclusion, although 3 CD19-CD3 BiTE recombinant protein had different CD19 binding affinity, similar binding capacity were determined for secreted BiTEs using 1 μg BiTE mRNA to transfect Hek293 for 48 h. At least three mRNA designs were tested. HTX-01-001 mRNA comprises SEQ ID NOs: 1-4. HTX-01-002 mRNA comprises SEQ ID NOs: 5-8. HTX-01-003 mRNA comprises SEQ ID NOs: 7-10.

Analysis of mRNA-Encoded CD19-CD3 BiTE Mediate T Cell Killing of Cancer Cells.

As for CD3 engager BsAbs, induced T cell lysis activity against tumor cells is the most important characteristic to assess their in vitro potency and specificity. We performed killing assays against CD19 positive tumor cells for mRNA-encoded CD19-CD3 BiTE using collected supernatant as described herein. For CD19 positive tumor cell line Raji, we evaluated T cell lysis activity using flow cytometry based killing assay. In general, Raji cells were first labeled using CellTrace Violet dye following the manufacturer's instructions and 20,000 cells per well were seeded in 96 well round bottom tissue culture plate. Resting human PBMCs were added into each well at E/T ratio of 5:1. 100 μl supernatants collected from BiTE mRNA transfected Hek293 cell were then added into the co-culture system and incubated for 20 hours. After treatment, cells were stained using fixable viability dye eFluor 780 (FVD-eFluor780,) following the manufacturer's instructions. The number of live target cells were measured using a gate of CellTrace Violet+FVD-eFluor780-. Killing activity was calculated as ([number of live target cells without treatment−number of live target cells with treatment]/[number of live target cells without treatment]×100%.

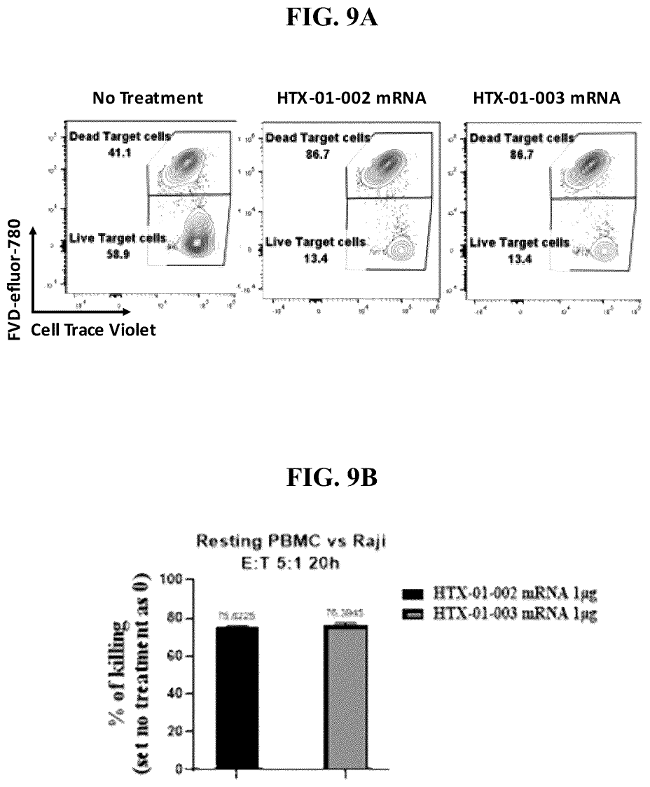

As shown in both FACs analysis (FIG. 9A) and calculated result after normalized against no treatment control (FIG. 9B), CD19-CD3 BiTEs which were produced by in vitro transfected encoding mRNAs can induce strong T cell killing activity against CD19 positive Raji cells.

In Vivo Screening of Cationic LNP Formulated CD19-CD3 BiTE Encoding mRNA.

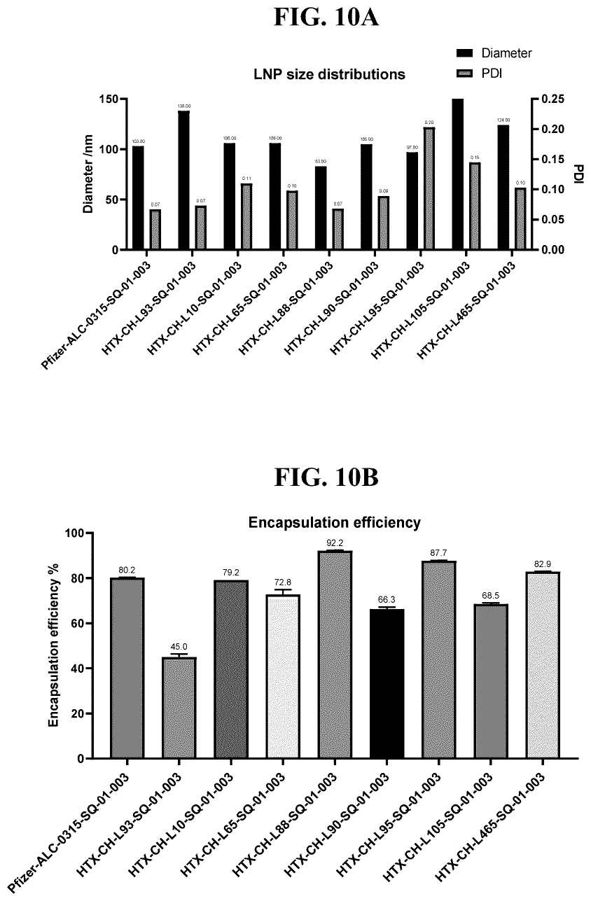

Nice types of lipid nanoparticles encapsulating mRNA encoding the CD19 BiTE (e.g., LNP-CD19/CD3 mRNA) were formulated as described in Example 2 and were characterized as described in Example 3. The particle size, polydispersity index (PDI), and encapsulation of 9 nanopariticles using different cationic lipids but same mRNA clone HTX-01-003 were characterized. LNPs were diluted in PBS pH=7.4 and transferred into a quartz cuvette (ZEN2112) to measure size by Dynamic Light Scattering (DLS) in the Zetasizer Nano ZS (Malvern Panalytical) using particle RI of 1.45 and absorption of 0.001 in PBS at 25° C. with viscosity of 0.888 cP and RI of 1.335. Measurements were made using a 1730 backscatter angle of detection previously equilibrated to 25° C. for 30 s in duplicates, each with 5 runs and 10 s run duration, without delay between measurements. Each measurement had a fixed position of 4.65 mm in the quartz cuvette with an automatic attenuation selection. As shown in FIG. 10A, the particle sizes of 9 different LNPs using same mRNA cargos were various as indicated, but all are in the range of 80-150 nM. And all LNPs were uniform with PDI under 0.15.

Encapsulation efficiency was done by using commercial Invitrogen™ Quant-it™ RiboGreen RNA Assay Kit. LNP formulations were diluted 250 times using TE buffer or Triton/TE (0.5% v/v Triton in TE buffer) and incubated at room temperature for 30 min to extract LNPs with Triton before adding to the 96 well plate. Microplates were then immediately introduced into the SpectraMax® iD3 plate reader to read fluorescence (Ex485/Em530). ?please add calculation formula for % of encapsulation efficiency. As shown in FIG. 10B, ideal encapsulation efficiencies were determined for majority LNPs under the same condition, especially the LNPs using HTX-88 (e.g., L88-CD19/CD3 mRNA) show the best encapsulation efficiency which was higher than 90%.

To evaluate the expression level of CD19-CD3 BiTE generation in vivo for cationic LNP formulations, Balb/c WT mice were used for this study. 10 μg of formulations described in example 6 were intravenous (i.v.) administration through tail veil. Plasma levels of CD19-CD3 BiTE were detected after 5 h of i.v injection using CD19 based ELISA quantification assay. The experiments were performed as same as mentioned in example 3 and analysis using purified HTX-01-003 BiTE protein as standard.

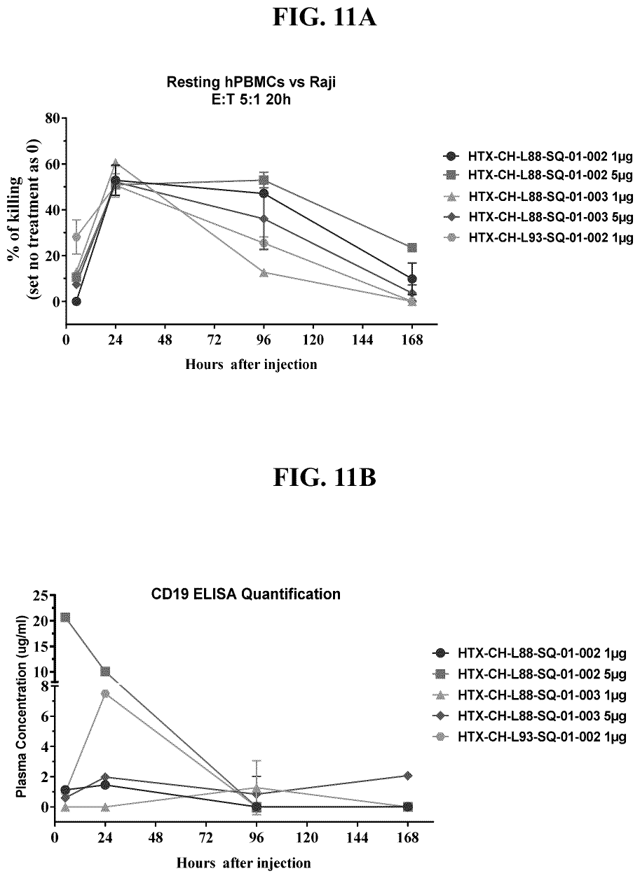

As shown in FIG. 11B, although formulated same amount mRNA, different cationic LNPs showed significant differences of mRNA delivery efficiency in vivo after 5 h injection. As shown in FIG. 11B, HTX-88 formulated CD19-CD3 BiTE encoding mRNA (e.g., L88-CD19/CD3 mRNA) showed the most endogenously protein expression among all 9 different HTX LNPs, including clinical approved LNPs of Pfizer's Covid-19 vaccine. The sufficient amount of protein was also translated from HTX-93 LNP delivered CD19-CD3 BiTE encoding mRNA (e.g., L93-CD19/CD3 mRNA). Those two cationic LNP were chosen to deliver different mRNA cargos for in-depth ex vivo analysis.

Ex Vivo Evaluation of LNP Formulated CD19-CD3 BiTE Encoding mRNA (LNP-CD19/CD3 mRNA)

To evaluate pharmacologically active levels of functional CD19-CD3 BiTE endogenously translated from LNP formulated CD19-CD3 BiTE encoding mRNA, plasma of single i.v. administration of L88-CD19/CD3 mRNA or L93-CD19/CD3 mRNA as indicated in BALB/c mice were collected at 5 h, 24 h, 96 h and 168 h post-injection. Both ex vivo killing activity and plasma concentration were evaluated.

As shown in FIG. 11B, plasma level of CD19-CD3 BiTE protein endogenously translated from the administered mRNA can be detected at 6 h and were sustain over several days especially for 5 μg mRNA dosing group. Accordingly, the ex vivo cytotoxic activity of plasma from the treatment mice showed maximum killing at 24 h with no significant difference between 5 and 1 μg dose group. However, unlike protein expression level in plasma dropped sharply 4d after injection, the strong ex vivo tumor cell killing activity was achieved with a single dosing of LNP formulated mRNA at the same timepoint. What's more, 4 of 5 treatment groups remain above the half-maximal killing activity at day 4, and single dosing of HTX-CH-L88 formulated with Sug HTX-01-002 mRNA maintained above the half-maximal killing activity for 7 days.

### Example 7. Efficacy of L88-CD19/CD3 mRNA in Animals

Study design is shown in TABLE 5.

TABLE 5
Study Design.

| | | | Dosing route & | # of |
|---|---|---|---|---|
| Groups | Treatment | Dosage | Frequency | mice |
| 1. G1 | PBS | N.A. | i.v., twice per week for | 5 |
| | | | total 5 doses | |
| 2. G2 | Recombinant BiTEs | 10 μg | i.v. 3 time per week for | 5 |
| | | | total 7 doses | |
| 3. G3 | LNP-CD19/CD3 | 1.6 μg | i.v., twice per week for | 5 |
| | mRNA | | total 5 doses | |

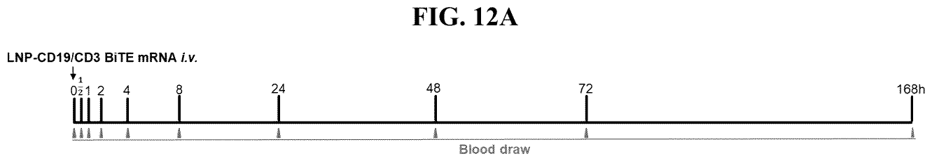

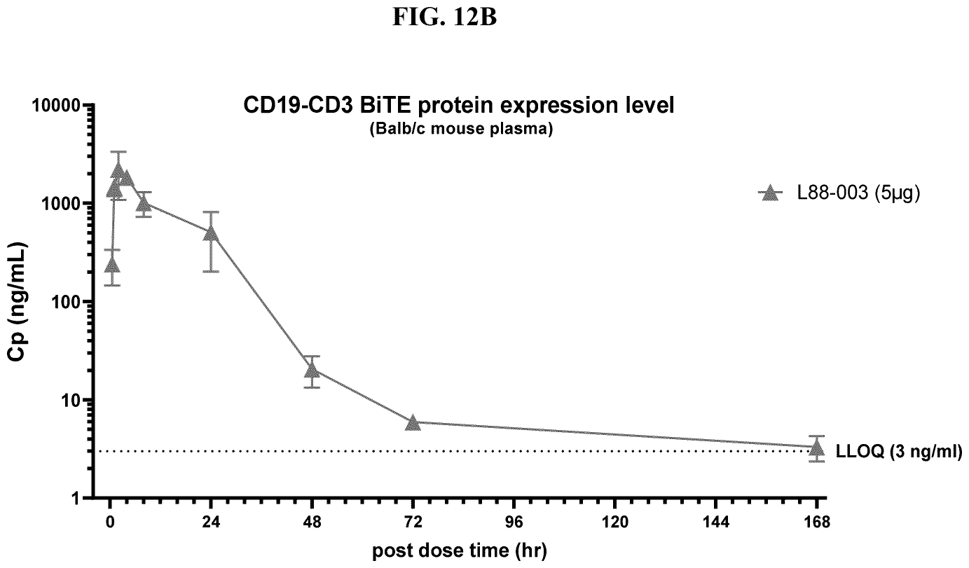

L88-003 (or referred to as LNP-CD19/CD3 mRNA hereafter) were used in this study. FIGS. 12A-12B show PK profiling of single dose LNP-CD19/CD3 mRNA in Balb/c mice. FIG. 12A is a schematic diagram of single dose PK study experimental design. As depicted in FIG. 12B, CD19-CD3 BiTE protein concentration in the plasma of Balb/c mice after i.v. administration of LNP-mRNA. Mice were treated intravenously with a dose of L88-CD19/CD3 mRNA comprising 5 μg CD19 BiTE mRNA. 5 mice were blood draw of each timepoint for quantification by CD3 ELISA assay. Concentrations from technical ELISA duplicates are shown. Data are presented as means±SD. PK parameters of plasma BiTE protein expressed after 5 μg L88-CD19/CD3 mRNA administration are calculated by WinNonlin (New Jersey, US) and included in TABLE. 6 below.

| | | Half | | | | Volume of | Clearance |
|---|---|---|---|---|---|---|---|
| | Lambda_z | Life | T_{MAX} | C_{MAX} | AUC | Distribution | (mL/(hrs)/ |
| R² | (1/(hrs)) | ((hrs)) | ((hrs)) | (ng/mL) | ((hrs)*ng/mL) | (mL/kg) | kg) |
| 0.97 | 0.09 | 7.93 | 2.00 | 2206.94 | 27959.80 | 102.35 | 8.94 |

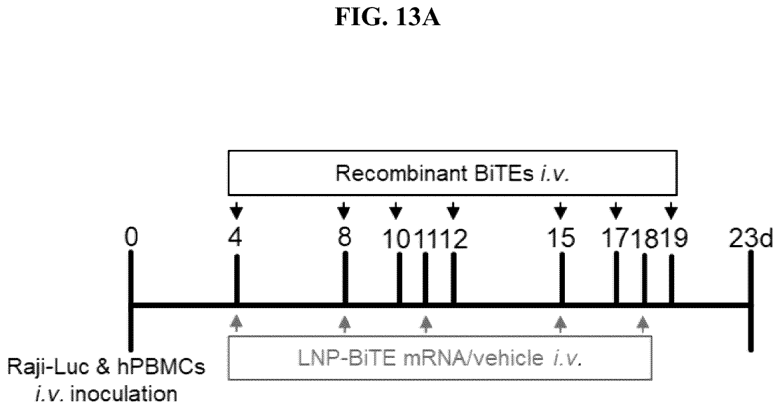

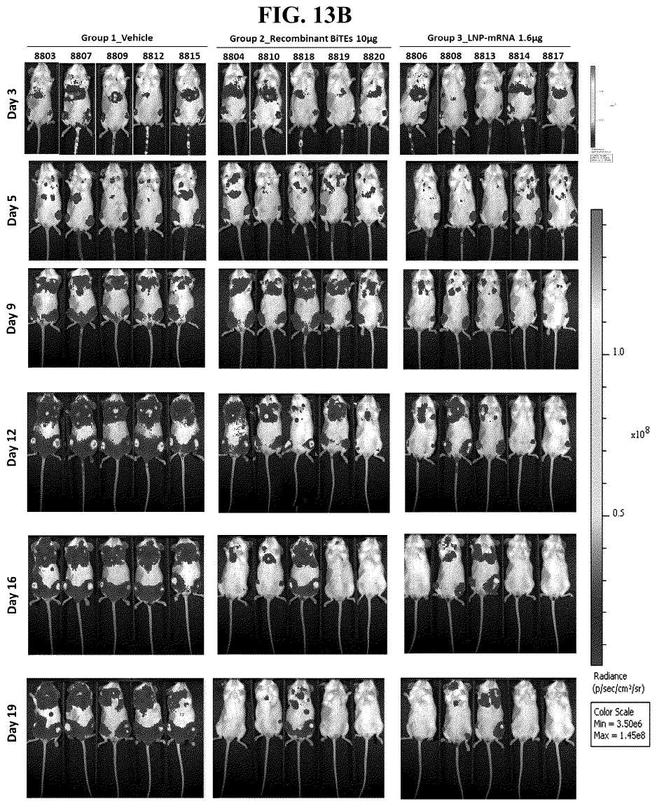

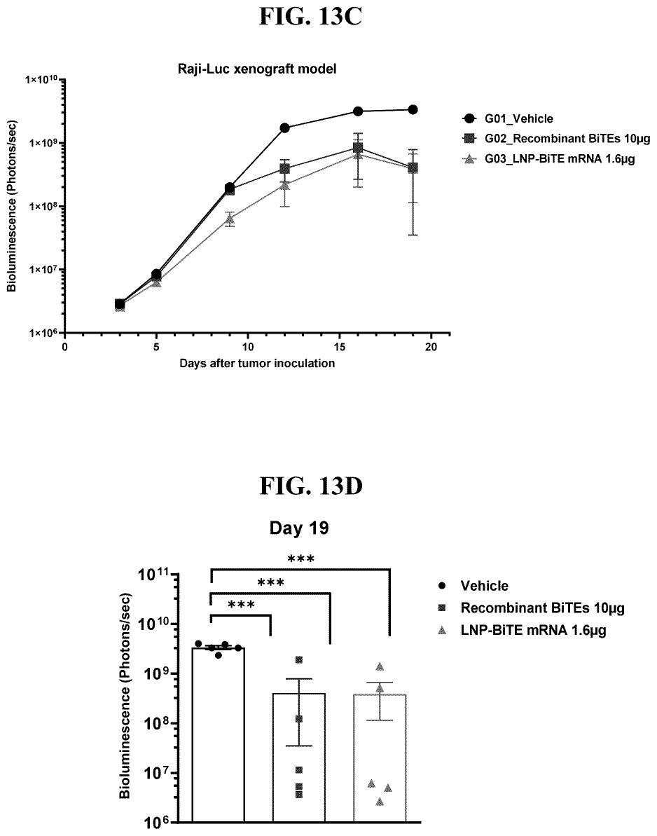

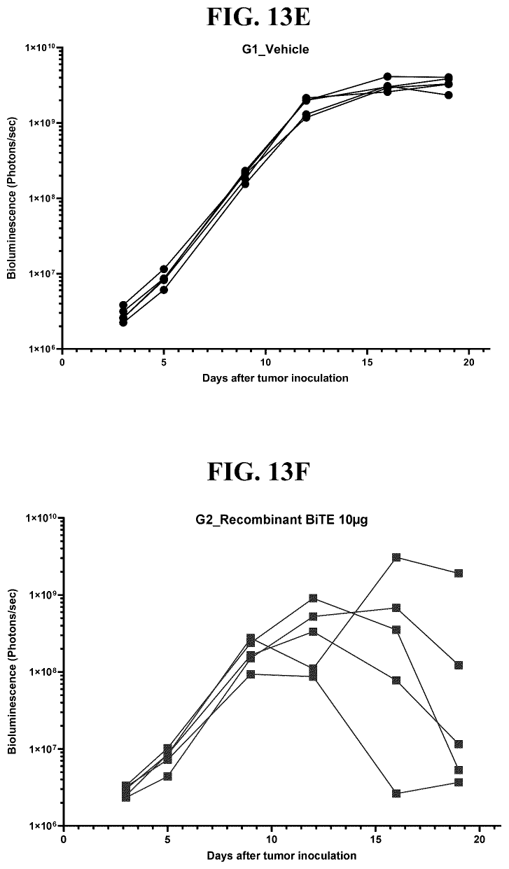

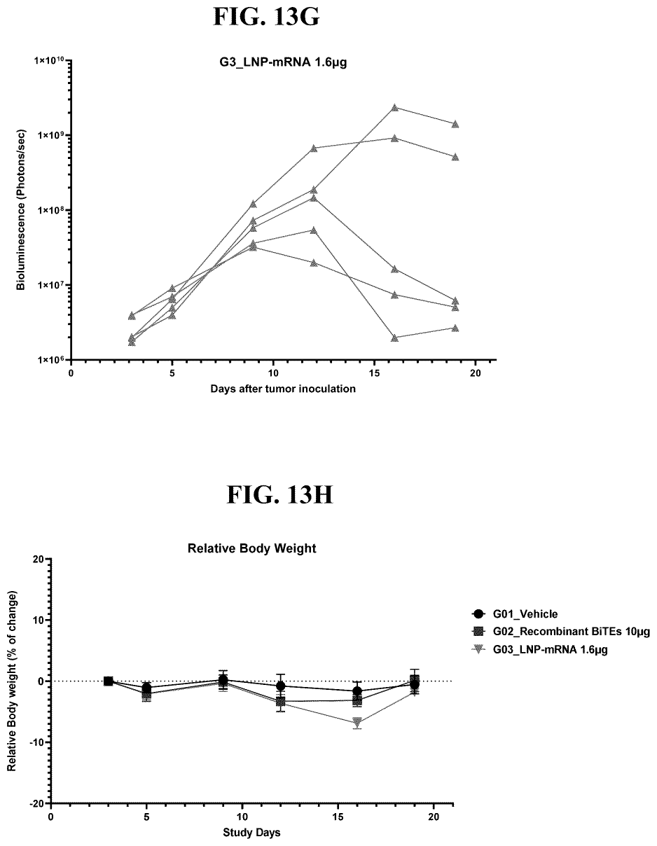

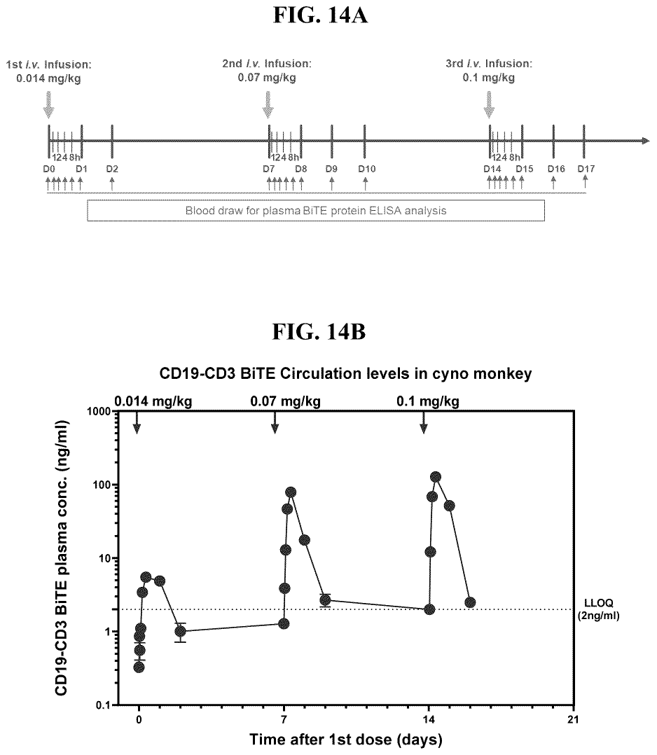

FIGS. 13A-13H depict results from the efficacy study of L88-003 (e.g., LNP-CD19/CD3 mRNA) performed in hPBMC-reconstituted Raji-Luciferase xenograft mouse model. FIG. 13A is the schematic diagram of study design with tumor cell inoculation and regiment of 3 treatment groups (n=5 for each group) including LNP-CD19/CD3 mRNA (L88-003, 1.6 μg of mRNA per mouse by intravenous injection), recombinant BiTE protein (rPR003, 10 μg of recombinant BiTE protein per mouse by intravenous injection), and vehicle. FIG. 13B depicts the total photon flux images of individual mice in each group that were captured at indicated timepoints by In Vivo Imaging System (IVIS). As depicted in FIGS. 13C-13G, LNP-CD19/CD3 mRNA achieved non-inferior results in xenograft tumor killing compared to recombinant BiTE protein. Additionally, LNP-CD19/CD3 mRNA was administered less frequently than recombinant BiTE protein, because of the longer half-lives of LNP-CD19/CD3 mRNA. Bioluminescence signal was quantitated as photons/sec using living Image 4.7 software for each treatment group. Individual total flux (TF) at end-timepoint of this study were analysis. Each dot represent bioluminescence signal of individual mouse and statistical analysis were compared to G1_Vehicle control group by Kruskal-Wallis test with Dunn's multiple comparisons test. *** p<0.01. E,F,G) TF changes of each mouse in individual groups during the treatment were analysis. As depicted in FIG. 13H, relative body weight changes of each mouse during the treatment were similar. Weight of mice were normalized to baseline timepoint (t=0) and each point represent as means±SD. FIGS. 14A-14B show the plasma CD19-CD3 BiTE protein PK profiling for escalated dose of IV infusion of L88-003 (e.g., LNP comprising mRNA encoding CD19/CD3 BiTE) in cynomolgus monkey. FIG. 14A is a schematic diagram of 3 escalation dose regiment and blood collection at indicated timepoints for PK profiling. L88-003 were dosed based on the body weights of the non-human primates. First dose of L88-003 used as 0.014 mg/kg. Second dose of L88-003 was 0.07 mg/kg, and the third dose was 0.1 mg/kg. FIG. 14B depicts CD19/CD3 BiTE protein concentration in the plasma of cynomolgus monkey at indicated timepoints after i.v. infusion of L88-003Proteins were quantified in a CD3 ELISA assay. Data are presented as means±SD of technical ELISA duplicates and dotted line depicts the LLOQ of 2 ng/ml. TABLE 7 lists pharmacokinetic parameters of BiTE protein expressed after administration of L88-003 analyzied by WinNoLin.

TABLE 7
Dose-dependent protein expression of LNP-BiTE mRNA in NHP

| Dose | Tmax | Cmax | t_{1/2} | AUCₗₐₛₜ | AUC_{INF} | Vz |
|---|---|---|---|---|---|---|
| (mg/kg) | (hours) | (ng/ml) | (hours) | (hr*ng/ml) | (hr*ng/ml) | (ml/kg) |
| 0.1 | 8 | 126.71 | 6.9 | 2202.6 | 2227.4 | 447 |
| 0.07 | 8 | 78.47 | 8.25 | 1159.6 | 1191.6 | 670 |

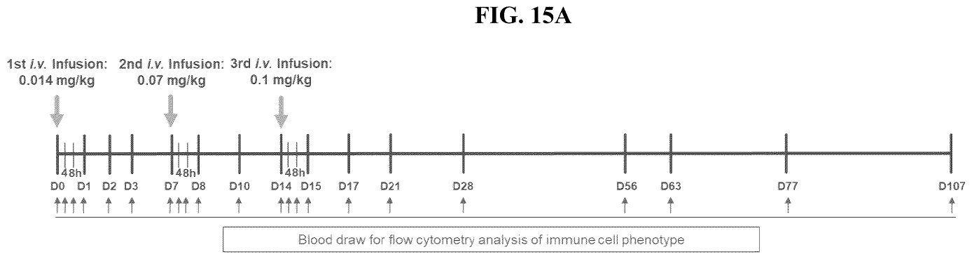

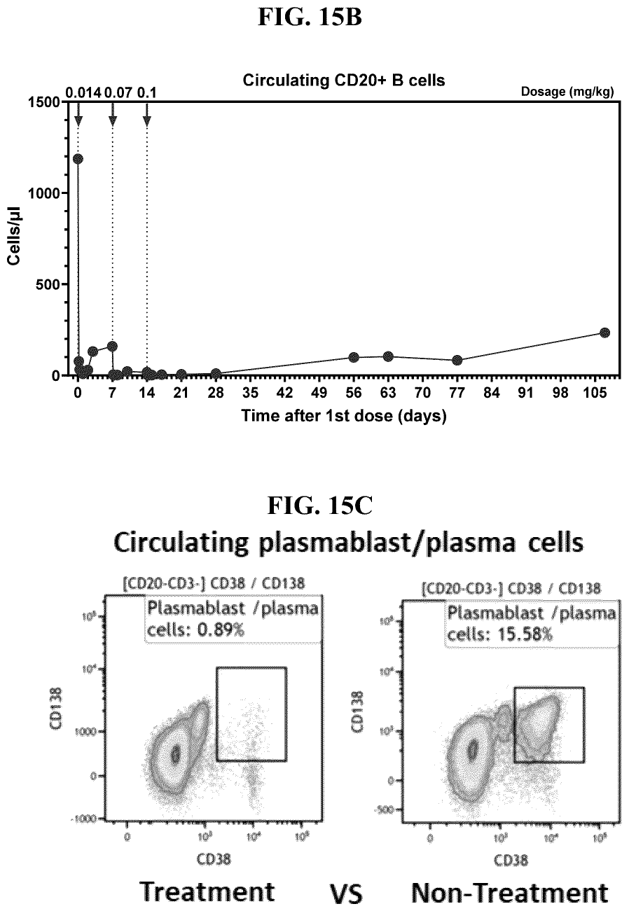

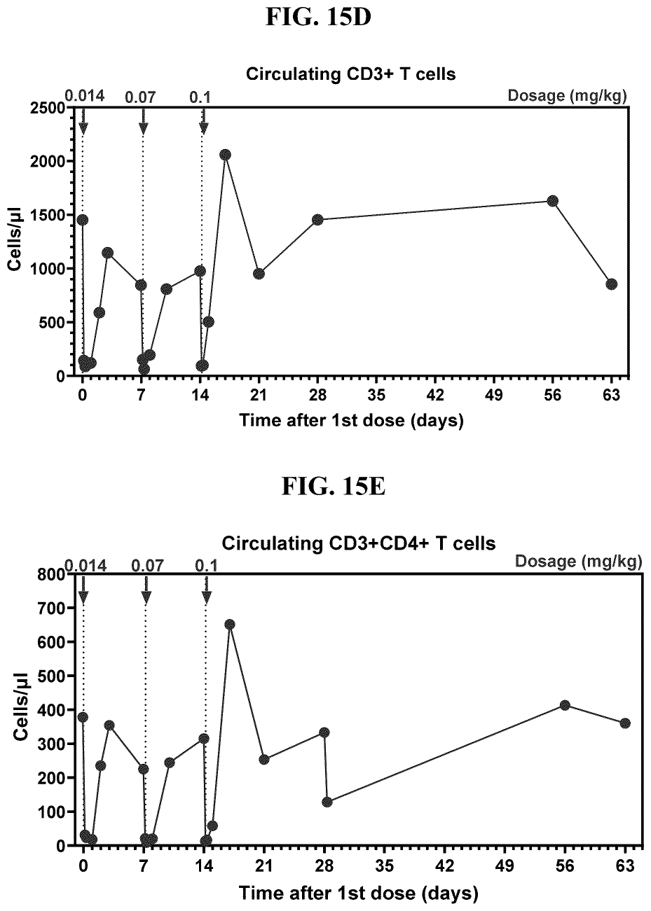

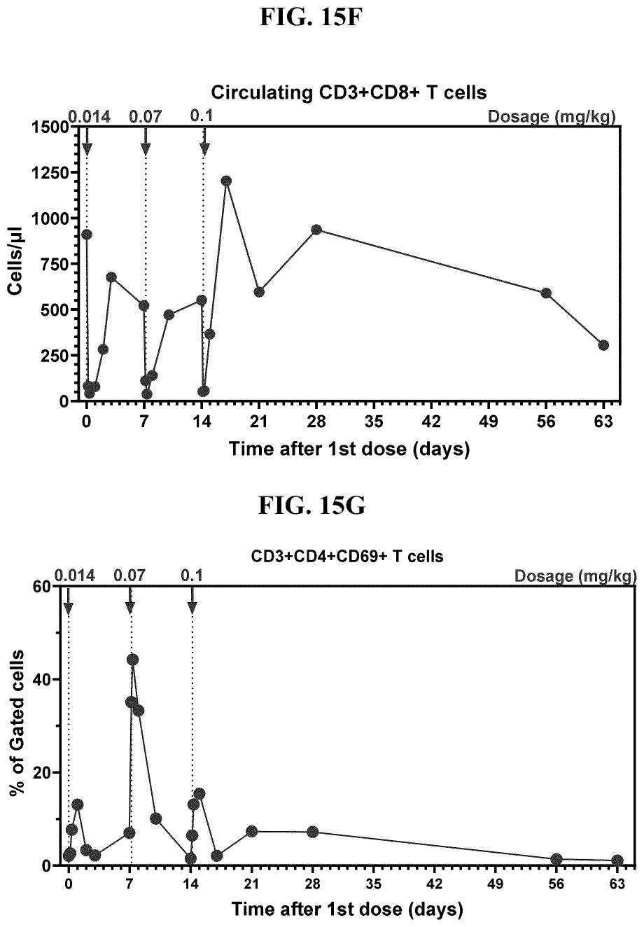

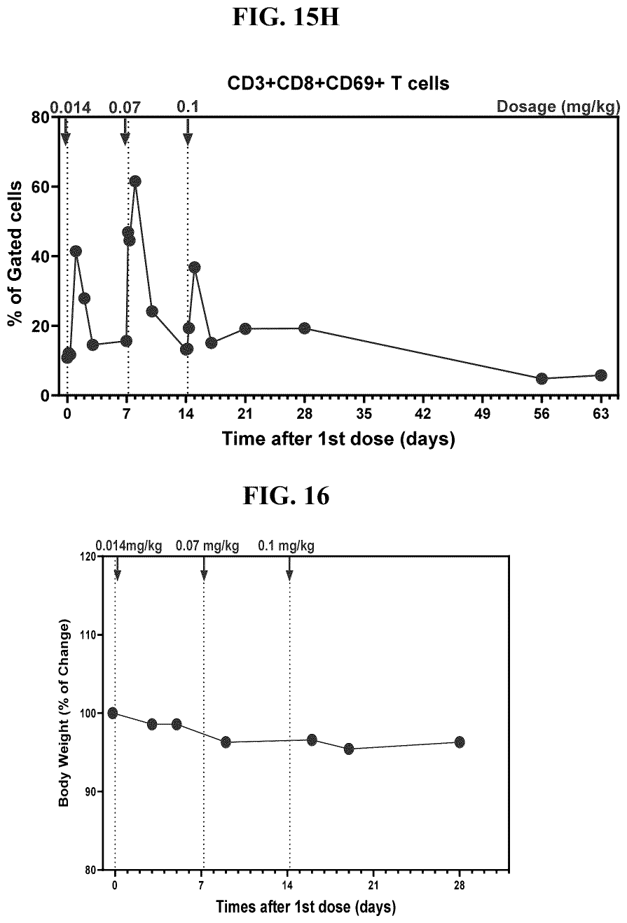

FIGS. 15A-15G show circulating B-cell depletion and T-cell dynamic analysis in cynomolgus monkey following 3 weekly escalated doses of L88-003 (e.g., LNP-CD19 BiTE mRNA). FIG. 15A is a schematic diagram of 3 escalation dose regiment and blood collection at indicated timepoints for peripheral B and T-cell phenotyping by flow cytometry. FIG. 15B and FIG. 15D show the absolute cell count of circulating CD20+ B-cells and circulating CD3+ T cells respectively at indicated timepoints. Circulating B cells were observed for over 100 days after administration. Prolonged 80% of circulating B cell depletion compared with pre-dose was observed for at least 13 weeks after last L88-003 dosing. The circulating B cell number started to recover after 5 weeks post final dosing. However, only about 20% of pre-dose B cell number was captured at 13 weeks post dosing, which indicated that the long lasting CD19+ memory B cells and plasmablasts were depleted in situ of second lymphoid tissues. Those CD19+ cells couldn't recover in short term after complete depletion. What's more, as depicted in FIG. 15C, compared with no treatment, the circulating plasmablast/plasma cells from L88-003 treated group were 17-fold lower at 105 days post treatment, indicating the long lasting, CD19+ plasmablasts, which are often considered harder to target and kill, were efficient depleted. All of those indicates that the LNP-CD19/CD3 mRNA compositions were successfully delivered to second lymphoid tissues (e.g., spleen, lymph nodes, bone marrow etc.), in addition to the liver, and resulted in superior efficacy of the in situ BiTE production for killing all CD19+ cell types, including tissue resident memory B cells and plasmablasts. FIG. 15E and FIG. 15F show the absolute cell count of circulating CD3+CD4+T helper cells and CD3+CD8+CTLs at indicated timepoints respectively. FIG. 15G and FIG. 15H show the % of CD69+ activated T-cells in CD4 and CD8 T-cell subgroups respectively. Treatment of L88-003 was associated with transient fluctuation of both circulating CD4+ and CD8+ T cells. Contrary to the durable reduction in B cells, T lymphocytes recovered quickly after each infusion and reached or exceeded baseline levels before next dose. And L88-003 treatment was also associated with transient upregulation of T cell activation marker, CD69 in both CD4+ and CD8+ T-cell subsets. Therefore, L88-003 is capable of mediating T-cell activation in vivo.

FIG. 16 depicts the relative body weight changes monitored twice per week and normalized to baseline timepoint (t=0). No significant weight loss was observed in the animals and the LNP compositions were well tolerated.

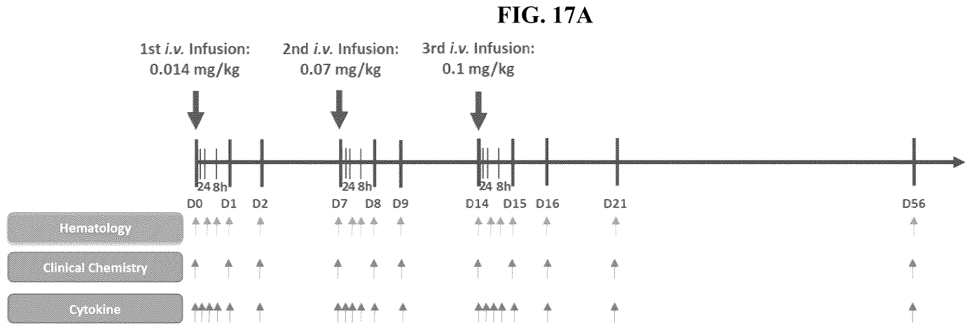

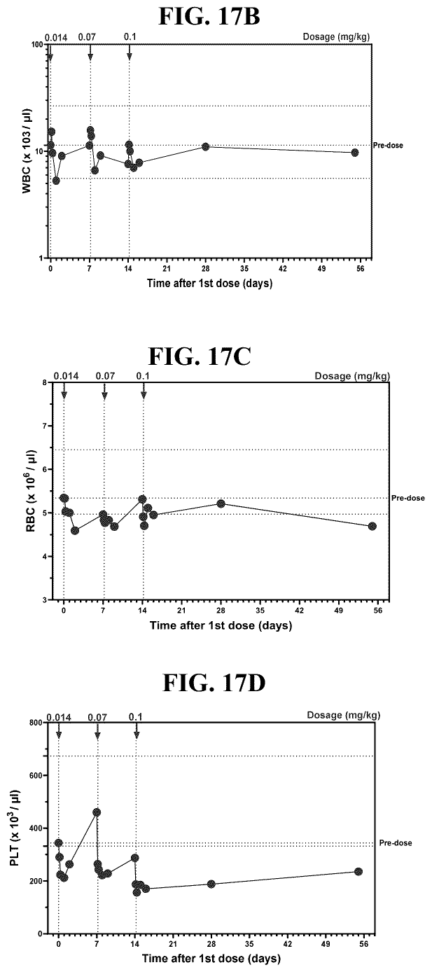

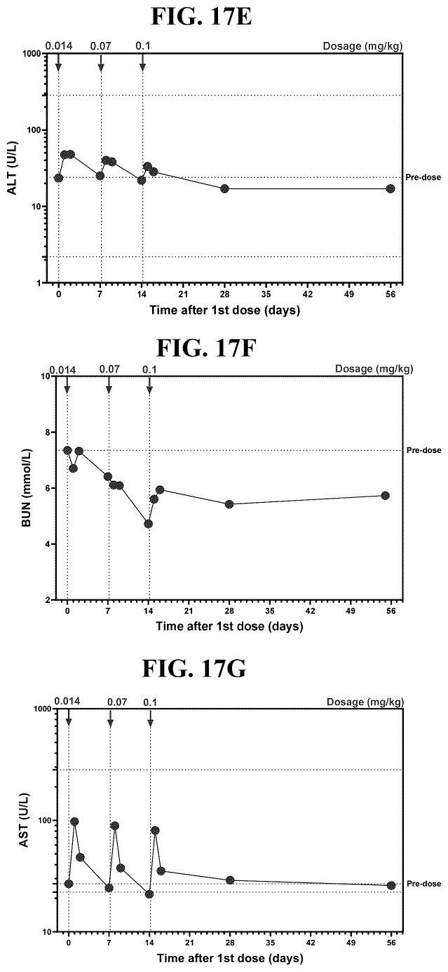

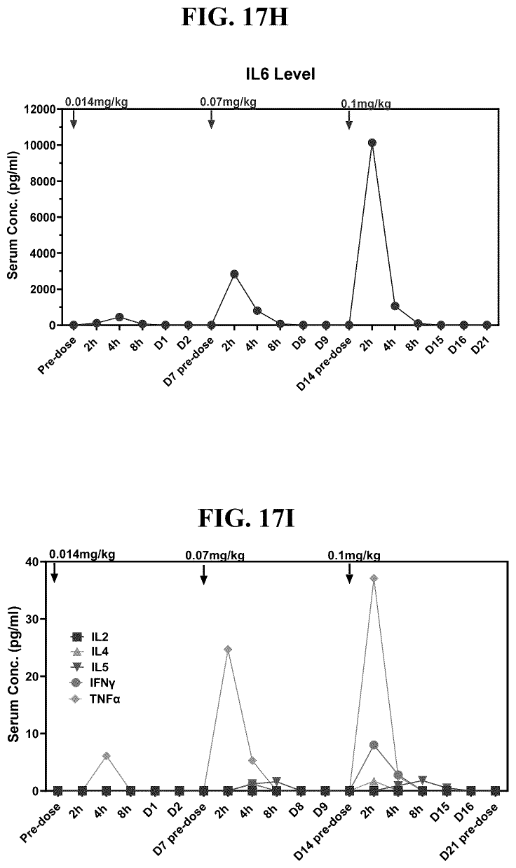

FIG. 17A-17I depicts results from the non-clinical tolerability study in cynomolgus monkey following 3 weekly escalated doses of L88-003 (e.g., LNP-CD19 BiTE mRNA). FIG. 17A is a schematic diagram of 3 escalation dose regiment and blood collection at indicated timepoints for clinical hematology, clinical chemistry, and cytokine analysis. FIGS. 17B-17D show whole blood CBC hematology analysis evaluated at indicated timepoints. Absolute counts of white blood cells (WBC), red blood cells (RBC), and platent (PLT) graphed in FIG. 17B, FIG. 17C, and FIG. 17D respectively. The dotted lines depict the reference range and/or pre-dose readouts. FIGS. 17E-17G show clinical chemistry analysis detected at indicated timepoints. Serum concentration of alanine aminotransferase (ALT), aspartate aminotransferase (AST), and urea nitrogen (BUN) graphed respectively in FIG. 17E, FIG. 17F, and FIG. 17G. Only transient elevation in liver enzymes were observed and the LNP compositions were well tolerated. The dotted lines depict the reference range and/or pre-dose readouts. FIG. 17H and FIG. 17I show plasma cytokine levels including IL6, IL2, IL4, IFNγ, and TNFα levels quantified by multiplex luminex assay. Only transient elevation in cytokines were observed and the LNP compositions were well tolerated.

1. A pharmaceutical composition, comprising:
a) a synthetic nucleic acid molecule encoding a therapeutic peptide, wherein the therapeutic peptide is configured to bind to a B-cell antigen; and
b) a plurality of lipid nanoparticles;
wherein the synthetic nucleic acid molecule is encapsulated within at least one of the plurality of lipid nanoparticles.

2. (canceled)

3. (canceled)

4. The pharmaceutical composition of claim 1, wherein the B-cell antigen comprises CD19.

5. (canceled)

6. The pharmaceutical composition of claim 1, wherein the therapeutic peptide binds to a second antigen.

7. (canceled)

8. (canceled)

9. The pharmaceutical composition of claim 6, wherein the second antigen comprises CD3 and the therapeutic peptide is configured to bind to CD3.

10. The pharmaceutical composition of claim 1, wherein the therapeutic peptide comprises an antibody or an antigen-binding fragment thereof.

11. (canceled)

12. The pharmaceutical composition of claim 10, wherein the bispecific antibody or the antigen-binding fragment thereof comprises a bispecific T-cell engager (BiTE).

13.-18. (canceled)

19. The pharmaceutical composition of claim 1, wherein the synthetic nucleic acid molecule comprises a synthetic ribonucleic acid (RNA) sequence.

20.-22. (canceled)

23. The pharmaceutical composition of claim 19, wherein the synthetic RNA comprises, in a 5′ to 3′ direction:
a) a 5′ untranslated region (5′ UTR) coding region;
b) a signal peptide coding region;
c) a therapeutic peptide coding region;
d) a 3′ untranslated region (3′ UTR) coding region; and
e) a poly A tail coding region.

24. The pharmaceutical composition of claim 23, wherein the signal peptide coding region comprises the sequence of SEQ ID NO: 11.

25. (canceled)

26. (canceled)

27. The pharmaceutical composition of claim 23, wherein the therapeutic peptide coding region encodes a variable light (VL) chain by a sequence that is at least 90%, 95%, 98%, or 99% identical to SEQ ID NOS: 1, 3, 5, 7, or 9; and a variable heavy (VH) chain encoding by a sequence that is at least 90%, 95%, 98%, or 99% identical to SEQ ID NOS: 2, 4, 6, 8, or 10.

28. The pharmaceutical composition of claim 23, wherein the therapeutic peptide coding region comprises nucleic acid sequences having at least 90%, 95%, 98%, or 99% sequence identity to 1) SEQ ID NOs: 1 and 2; 2) SEQ ID NOs: 5 and 6; or 3) SEQ ID NOs: 9 and 10, wherein the nucleic acid sequences encode an anti-CD19 antibody or anti-CD19 binding fragments thereof.

29. (canceled)

30. The pharmaceutical composition of claim 23, wherein the therapeutic peptide coding region comprises nucleic acid sequences having at least 90%, 95%, 98%, or 99% sequence identity to 1) SEQ ID NOs: 3 and 4; or 2) SEQ ID NOs: 7 and 8, wherein the nucleic acid sequences encode an anti-CD3 antibody or anti-CD3 binding fragments thereof.

31. (canceled)

32. (canceled)

33. The pharmaceutical composition of claim 23, wherein the therapeutic peptide coding region comprises, in a 5′ to 3′ direction:
a) an anti-CD19 light chain coding region;
b) an anti-CD19 heavy chain coding region;
c) an anti-CD3 heavy chain coding region; and
d) an anti-CD3 light chain coding region.

34. The pharmaceutical composition of claim 33, wherein the anti-CD19 light chain coding region comprises the sequence of SEQ ID NOs: 1, 3, 5, or 9; and wherein the anti-CD19 heavy chain coding region comprises the sequence of SEQ ID NOs: 2, 4, 6, or 10.

35.-80. (canceled)

81. The pharmaceutical composition of claim 1, wherein the lipid nanoparticle comprises an ionizable lipid or a pharmaceutically acceptable salt thereof:
wherein the ionizable lipid comprises an amine head group and at least one hydrophobic tail
R^{Lipid} having a structure of 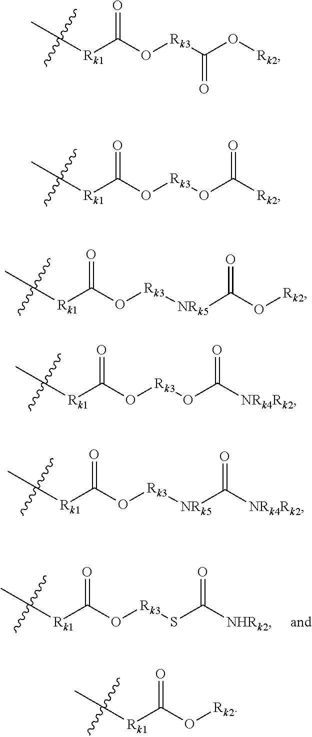
or a pharmaceutically acceptable salt thereof; further wherein
Rₖ₁ is independently a C1-C12 bivalent aliphatic or heteroaliphatic radical;
Rₖ₃ is independently a C1-C12 alkyl, C2-C12 alkenyl, C2-C12 alkynyl, C3-C12 cycloalkyl, C3-C12 heterocycloalkyl, aryl, or heteroaryl;
Rₖ₂ is independently a C1-C20 alkyl, C2-C20 alkenyl, C2-C20 alkynyl, C3-C20 cycloalkyl, C1-C20 heteroalkyl, C3-C20 heterocycloalkyl, aryl, or heteroaryl;
Rₖ₄ and Rₖ₅ are each independently H or C1-C12 bivalent aliphatic radical; and
M is O or NRₖ₆, wherein Rₖ₆ is H or C1-C12 aliphatic radical.

82. The pharmaceutical composition of claim 1, wherein the amine head group is represented by: 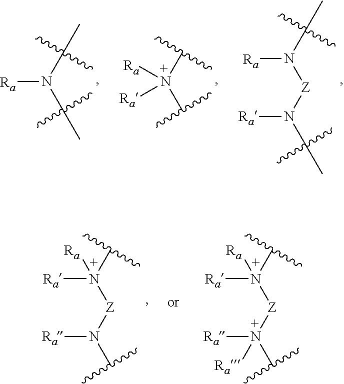
wherein Ra, Ra′, Ra″, and Ra′″ are each independently, H, C1-20 alkyl, C2-C20 alkenyl, C2-C20 alkynyl, C3-C20 cycloalkyl or heterocycloalkyl, C1-C20 heteroalkyl, C3-C20 aryl or heteroaryl, or a R^{Lipid}; and
Z is a C1-C20 bivalent aliphatic radical, a C1-C20 bivalent heteroaliphatic radical, a bivalent aryl radical, or a bivalent heteroaryl radical.

83. (canceled)

84. The pharmaceutical composition of claim 81, wherein the amine head group is selected from the group consisting of

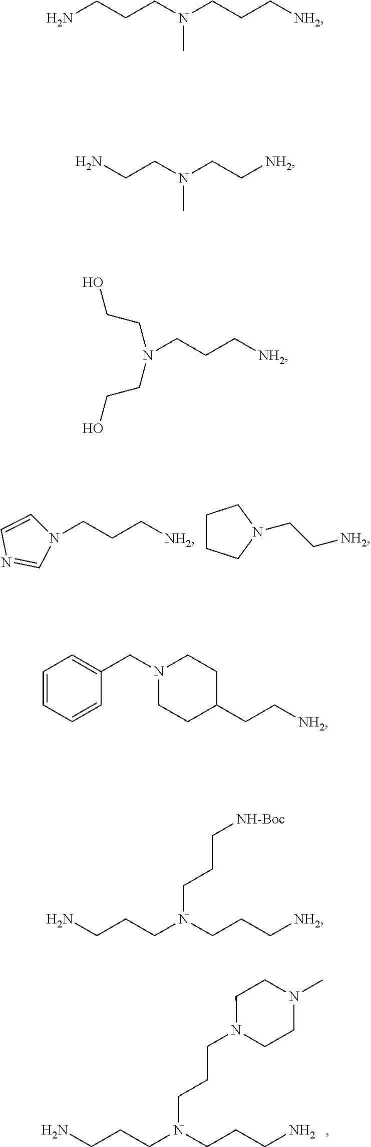

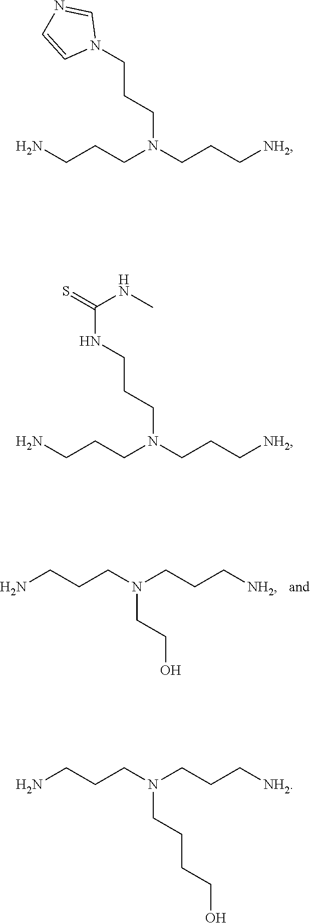 [SMILES: NCCCN(CCCN)CCCn1ccnc1 ; CNC(=S)NCCCN(CCCN)CCCN ; NCCCN(CCO)CCCN ; "and" ; NCCCN(CCCN)CCCCO]

85. The pharmaceutical composition of claim 81, wherein the at least one hydrophobic tail comprises,

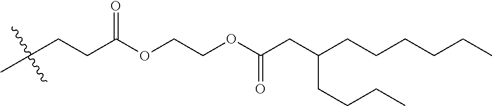 [SMILES: *CCC(=O)OCCOC(=O)CC(CCCC)CCCCCC]

86. The pharmaceutical composition of claim 81, wherein the ionizable lipid comprises

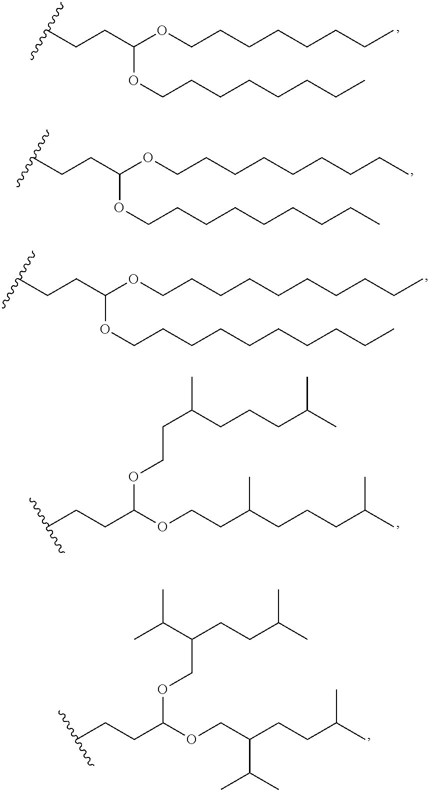

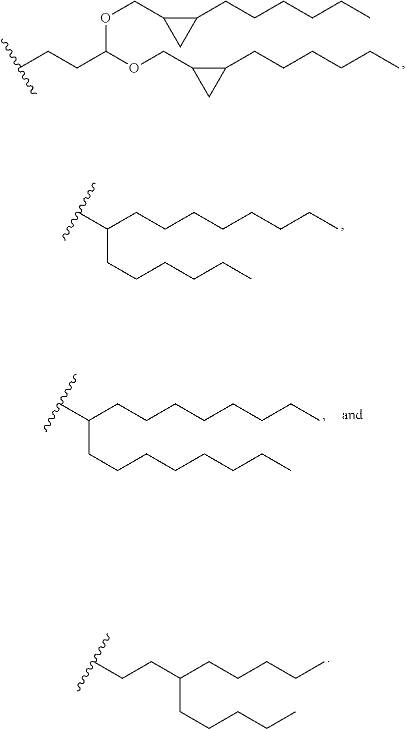 [SMILES: *CCC(OCC1CC1CCCCCC)OCC1CC1CCCCCC ; *C(CCCCCC)CCCCCCCC ; *C(CCCCCCCC)CCCCCCCC ; *CCC(CCCCC)CCCCC]

87-123. (canceled)

124. A method for treating a blood malignancy, comprising administering an effective amount of a pharmaceutical composition of claim 1 to a subject in need thereof, wherein the subject in need thereof has a blood malignancy.

125.-147. (canceled)
